SEQUENCE LISTING (20 sequences)

SEQ ID NO: 1 - protein, 669 aa, synthetic construct
SGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSARSRGIVEECCFRSCDLALLETYCATPAKSE

SEQ ID NO: 2 - protein, 669 aa, synthetic construct
SGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSQRSRGIVEECCFRSCDLALLETYCATPAKSE

SEQ ID NO: 3 - protein, 669 aa, synthetic construct
SGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSRRSAGIVEECCFRSCDLALLETYCATPAKSE

SEQ ID NO: 4 - protein, 669 aa, synthetic construct
SGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSRRSQGIVEECCFRSCDLALLETYCATPAKSE

SEQ ID NO: 5 - protein, 675 aa, synthetic construct
RGVFRRSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSARSRGIVEECCFRSCDLALLETYCATPAKSE

SEQ ID NO: 6 - protein, 675 aa, synthetic construct
RGVFRRSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSQRSRGIVEECCFRSCDLALLETYCATPAKSE

SEQ ID NO: 7 - protein, 675 aa, synthetic construct
RGVFRRSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSRRSAGIVEECCFRSCDLALLETYCATPAKSE

SEQ ID NO: 8 - protein, 675 aa, synthetic construct
RGVFRRSGDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSAYRPSETLCGGELVDTLQFVCGDRGFYFSRPASRVSRRSQGIVEECCFRSCDLALLETYCATPAKSE

SEQ ID NO: 9 - protein, 67 aa, synthetic construct
AYRPSETLCGGELVDTLQFVCGDRGFYFSRPASRVSARSRGIVEECCFRSCDLALLETYCATPAKSE

SEQ ID NO: 10 - protein, 67 aa, synthetic construct
AYRPSETLCGGELVDTLQFVCGDRGFYFSRPASRVSQRSRGIVEECCFRSCDLALLETYCATPAKSE

SEQ ID NO: 11 - protein, 67 aa, synthetic construct
AYRPSETLCGGELVDTLQFVCGDRGFYFSRPASRVSRRSAGIVEECCFRSCDLALLETYCATPAKSE

SEQ ID NO: 12 - protein, 67 aa, synthetic construct
AYRPSETLCGGELVDTLQFVCGDRGFYFSRPASRVSRRSQGIVEECCFRSCDLALLETYCATPAKSE

SEQ ID NO: 13 - protein, 67 aa, Homo sapiens
AYRPSETLCGGELVDTLQFVCGDRGFYFSRPASRVSRRSRGIVEECCFRSCDLALLETYCATPAKSE

SEQ ID NO: 14 - protein, 89 aa, Homo sapiens
RDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 15 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 16 - protein, 17 aa, Homo sapiens
RGFYFSRPASRVSRRSR

SEQ ID NO: 17 - protein, 585 aa, Homo sapiens [sequence omitted]

SEQ ID NO: 18 - protein, 4 aa, synthetic construct
GSGS

SEQ ID NO: 19 - protein, 5 aa, synthetic construct
GSGSA

SEQ ID NO: 20 - protein, 15 aa, synthetic construct
GGGGSGGGGSGGGGS

(19) United States
(12) Patent Application Publication
(10) Pub. No.: US 2026/0201016 A1
(43) Pub. Date: Jul. 16, 2026

(54) IGF2 FUSION PROTEIN FORMULATIONS AND THERAPEUTIC USES THEREOF

(71) Applicant: Juvena Therapeutics, Inc., Redwood City, CA (US)

(72) Inventor: Jeremy O’CONNELL, Palo Alto, CA (US)

(21) Appl. No.: 19/558,327

(22) Filed: Mar. 5, 2026

Related U.S. Application Data
(63) Continuation of application No. PCT/US2024/045683, filed on Sep. 6, 2024.
(60) Provisional application No. 63/581,262, filed on Sep. 7, 2023.

(51) Int. Cl. C07K 14/765; A61K 9/00; A61K 38/00; A61K 47/02; A61K 47/10; A61K 47/36; A61P 21/00; C07K 14/65

(52) U.S. Cl. CPC C07K 14/765; A61K 9/0019; A61K 47/02; A61K 47/10; A61K 47/36; A61P 21/00; C07K 14/65; A61K 38/00; C07K 2319/00

(57) ABSTRACT

Provided herein, in certain aspects, are pharmaceutical compositions comprising an HSA-IGF2 fusion protein formulated for subcutaneous administration. As provided herein, are methods of treating muscle disorders, including myotonic dystrophies, comprising administration of a pharmaceutical composition comprising an HSA-IGF2 fusion protein formulated for subcutaneous administration.

### CROSS-REFERENCE TO RELATED APPLICATIONS

This application is a continuation of International Patent Application No. PCT/US2024/045683, filed on Sep. 6, 2024, which claims the benefit of U.S. Provisional Patent Application No. 63/581,262, filed on Sep. 7, 2023, which are hereby incorporated by reference in their entireties.

### STATEMENT OF FEDERALLY SPONSORED RESEARCH

This invention was made with government support under R43 AG071181 awarded by the National Institutes of Health. The government has certain rights in the invention.

### INCORPORATION BY REFERENCE OF SEQUENCE LISTING

The present application is being filed along with a Sequence Listing in electronic format. The Sequence Listing is provided as a file entitled 54275-710_301_SL.xml, created on Mar. 5, 2026, which is 24,632 bytes in size. The information in the electronic format of the Sequence Listing is incorporated by reference in its entirety.

### TECHNICAL FIELD

Described herein are IGF2 fusion protein pharmaceutical compositions and formulations and methods for use of the pharmaceutical compositions and formulations for treatment of various muscle disorders including myotonic muscular dystrophy.

### BACKGROUND

Myotonic dystrophy is a genetic condition which can lead to multiorgan system dysfunction including progressive muscle weakness and muscle wasting. Pathogenesis of myotonic dystrophy can affect muscles of movement and can disrupt electrical conduction systems of the heart, breathing and swallowing muscles, gastrointestinal system, lens of the eyes, and central nervous system, among other symptomatology. Neurological symptoms, reproductive system dysfunction, and metabolic and hormonal changes often manifest as part of myotonic dystrophy.

Current treatment options for patients with myotonic dystrophy are limited. Treatments attempted to ameliorate symptoms have met with inadequate success. Currently there are no approved disease-modifying therapies for Myotonic Dystrophy Type 1 (DM1) or treatment strategies for DM1 that consistently and effectively manage symptoms. There remains a need to develop new treatments for myotonic dystrophies that protect against development and progression of pathogenic features of the disorders. There also remains a need to develop disease-modifying treatments that can improve symptoms in patients suffering from a myotonic dystrophy.

### SUMMARY

Described here are pharmaceutical compositions comprising a fusion protein comprising the amino acid sequence of SEQ ID NO: 1 and a stabilizer. In some aspects, the stabilizer is present at a mass concentration of solution of about 0.5% to 15% weight/volume (w/v) of the pharmaceutical composition. In some aspects, the stabilizer is present at a mass concentration of solution of at least about 5% w/v of the pharmaceutical composition. In some aspects, the stabilizer is present at a mass concentration of solution of less than about 10% w/v of the pharmaceutical composition. In some embodiments, the stabilizer is present at a mass concentration of solution between about 5 to 10% w/v of the pharmaceutical composition. In some embodiments, the stabilizer is present at a mass concentration of solution about 8% w/v of the pharmaceutical composition. In some embodiments, the stabilizer is a carbohydrate. In some embodiments, the carbohydrate comprises sucrose, mannitol, trehalose, raffinose, stachyose, xylitol, starch, verbascose, or any combination thereof. In some embodiments, the carbohydrate comprises sucrose. In some embodiments, sucrose is present at a mass concentration of solution between about 5 to 10% w/v of the pharmaceutical composition. In some embodiments, sucrose is present at a mass concentration of solution of about 8% w/v of the pharmaceutical composition.

Described here are pharmaceutical compositions comprising a fusion protein comprising the amino acid sequence of SEQ ID NO: 1 and a surfactant. In some aspects, the surfactant is present at a mass concentration of solution of about 0.001% to 1% w/v of the pharmaceutical composition. In some aspects, the surfactant is present at a mass concentration of solution of at least about 0.01% w/v of the pharmaceutical composition. In some aspects, the surfactant is present at a mass concentration of solution of less than about 1% w/v of the pharmaceutical composition. In some embodiments, the surfactant is present at a mass concentration of solution between about 0.05-0.20% w/v of the pharmaceutical composition. In some embodiments, the surfactant is present at a mass concentration of solution between about 0.01% w/v of the pharmaceutical composition. In some embodiments, the surfactant is an ionic surfactant. In some embodiments, the surfactant is a nonionic surfactant. In some embodiments, the nonionic surfactant comprises a polysorbate or a poloxamer. In some embodiments, the nonionic surfactant comprises a polysorbate comprising Tween 20, Tween 40, Tween 60, Tween 80, Tween 65, Tween 80, or any combination thereof. In some embodiments, the nonionic surfactant comprises a poloxamer comprising poloxamer 124, poloxamer 188, poloxamer 237, poloxamer 338, poloxamer 407, or any combination thereof. In some embodiments, the nonionic surfactant comprises poloxamer 188. In some embodiments, the nonionic surfactant comprises poloxamer 188 at a mass concentration of solution of about 0.1% w/v. In some embodiments, the nonionic surfactant consists of poloxamer 188.

Described here are pharmaceutical compositions comprising a fusion protein comprising the amino acid sequence of SEQ ID NO: 1 and a buffer. In some aspects, the buffer is present at a concentration of about 1 mM to about 100 mM. In some embodiments, the buffer comprises sodium, potassium, calcium, or ammonium. In some embodiments, the buffer comprises sodium and wherein the sodium is present at a concentration of about 3 mM to about 10 mM. In some embodiments, the buffer comprises sodium and wherein the sodium is present at a concentration of about 7 nM. In some embodiments, the pharmaceutical composition further comprises a surfactant. In some embodiments, the surfactant is an ionic surfactant. In some embodiments, the surfactant is a nonionic surfactant. In some embodiments, the surfactant is present at a concentration of about 0.001-1% w/v of the pharmaceutical composition. In some embodiments, the surfactant is present at a concentration of about 0.1% w/v of the pharmaceutical composition. In some embodiments, the nonionic surfactant comprises a polysorbate or a poloxamer. In some embodiments, the nonionic surfactant comprises a poloxamer comprising poloxamer 124, poloxamer 188, poloxamer 237, poloxamer 338, poloxamer 407, or any combination thereof. In some embodiments, the nonionic surfactant comprises poloxamer 188. In some embodiments, the nonionic surfactant comprises poloxamer 188 at a mass concentration of solution of about 0.1% w/v. In some embodiments, the pharmaceutical composition further comprises a stabilizer, wherein the stabilizer is present at a mass concentration of solution of about 0.5% to 15% weight/volume (w/v) of the pharmaceutical composition. In some embodiments, the pharmaceutical composition further comprises a stabilizer, wherein the stabilizer is present at a mass concentration of solution of at least about 5% w/v of the pharmaceutical composition. In some embodiments, the pharmaceutical composition further comprises a stabilizer, wherein the stabilizer is present at a mass concentration of solution of less than about 10% w/v of the pharmaceutical composition. In some embodiments, the stabilizer is present at a mass concentration of solution between about 5 to 10% w/v of the pharmaceutical composition. In some embodiments, the stabilizer is present at a mass concentration of solution about 8% w/v of the pharmaceutical composition. In some embodiments, the stabilizer is a carbohydrate. In some embodiments, the carbohydrate comprises sucrose, mannitol, trehalose, raffinose, stachyose, xylitol, starch, verbascose, or any combination thereof. In some embodiments, the carbohydrate comprises sucrose. In some embodiments, sucrose is present at a mass concentration of solution between about 5 to 10% w/v of the pharmaceutical composition. In some embodiments, sucrose is present at a mass concentration of solution of about 8% w/v of the pharmaceutical composition. In some embodiments, the pharmaceutical composition further comprises a salt, wherein the salt is present at a concentration of about 1 mM to about 100 mM. In some embodiments, the salt comprises sodium, potassium, calcium, or ammonium. In some embodiments, the salt comprises sodium and wherein the sodium is present at a concentration of about 3 mM to about 10 mM. In some embodiments, the salt comprises sodium and wherein the sodium is present at a concentration of about 7 nM. In some embodiments, the pharmaceutical composition is formulated for subcutaneous administration. In some embodiments, the fusion protein is formulated in a dosage form of injectable liquid having a volume of about 0.1 mL to about 3 mL, or about 0.5 mL to about 2 mL, or about 0.1 mL to about 1 mL. In some embodiments, the fusion protein is present at a concentration of about 30 mg/mL to about 350 mg/mL. In some embodiments, the fusion protein is present at a concentration of about 100 mg/mL to about 200 mg/mL. In some embodiments, the fusion protein is present at a concentration of between about 10 mg/mL to about 100 mg/mL. In some embodiments, the fusion protein is present at a concentration of between about 20 mg/mL to about 60 mg/mL. In some embodiments, the fusion protein is present at a concentration of between about 30 mg/mL to about 50 mg/mL. In some embodiments, the fusion protein is present at a concentration of about 40 mg/mL.

In some embodiments, the pharmaceutical composition comprises: HSA-IGF2-R61A at a concentration of 40.0 mg/mL, tri-sodium citrate dihydrate at a concentration of 1.74 mg/mL, citric acid monohydrate at a concentration of 0.23 mg/mL, sucrose at a concentration of 80.0 mg/mL, and Poloxamer 188 at a concentration of 1.0 mg/mL.

Described here are pharmaceutical compositions comprising a fusion protein comprising the amino acid sequence of SEQ ID NO: 1; and a pharmaceutically acceptable excipient, carrier, or diluent; wherein the pharmaceutical composition is formulated for subcutaneous administration. In some embodiments, the fusion protein is formulated in a dosage form of injectable liquid having a volume of about 0.1 mL to about 3 mL, or about 0.5 mL to about 2 mL, or about 0.1 mL to about 1 mL. In some embodiments, the fusion protein is present at a concentration of about 30 mg/mL to about 350 mg/mL. In some embodiments, the fusion protein is present at a concentration of about 100 mg/mL to about 200 mg/mL.

Described here are pharmaceutical compositions comprising a fusion protein comprising the amino acid sequence of SEQ ID NO: 1, wherein the fusion protein is present at a concentration of about 30 mg/mL to about 350 mg/mL. In some aspects, the fusion protein is present at a concentration of about 100 mg/mL to about 200 mg/mL. In some embodiments, the pharmaceutical composition is formulated in a subcutaneous unit dosage form. In some embodiments, the pharmaceutical composition is formulated in a dosage form of injectable liquid having a volume of about 0.1 mL to about 3 mL, or about 0.5 mL to about 2 mL, or about 0.1 mL to about 1 mL. In some embodiments, the pharmaceutical composition further comprises a surfactant. In some embodiments, the surfactant is an ionic surfactant. In some embodiments, the surfactant is a nonionic surfactant. In some embodiments, the surfactant is present at a concentration of about 0.001-1% w/v of the pharmaceutical composition. In some embodiments, the surfactant is present at a concentration of about 0.1% w/v of the pharmaceutical composition. In some embodiments, the nonionic surfactant comprises a polysorbate or a poloxamer. In some embodiments, the nonionic surfactant comprises a poloxamer comprising poloxamer 124, poloxamer 188, poloxamer 237, poloxamer 338, poloxamer 407, or any combination thereof. In some embodiments, the nonionic surfactant comprises poloxamer 188. In some embodiments, the nonionic surfactant comprises poloxamer 188 at a mass concentration of solution of about 0.05 to about 0.15% w/v. In some embodiments, the nonionic surfactant comprises poloxamer 188 at a concentration of about 1.0 mg/mL. In some embodiments, the nonionic surfactant comprises poloxamer 188 at a mass concentration of solution of about 0.1% w/v. In some embodiments, the pharmaceutical composition further comprises a stabilizer, wherein the stabilizer is present at a mass concentration of solution of about 0.5% to 15% weight/volume (w/v) of the pharmaceutical composition. In some embodiments, the pharmaceutical composition further comprises a stabilizer, wherein the stabilizer is present at a mass concentration of solution of at least about 5% w/v of the pharmaceutical composition. In some embodiments, the pharmaceutical composition further comprises a stabilizer, wherein the stabilizer is present at a mass concentration of solution of less than about 10% w/v of the pharmaceutical composition. In some embodiments, the stabilizer is present at a mass concentration of solution between about 5 to 10% w/v of the pharmaceutical composition. In some embodiments, the stabilizer is present at a mass concentration of solution about 8% w/v of the pharmaceutical composition. In some embodiments, the stabilizer is a carbohydrate. In some embodiments, the carbohydrate comprises sucrose, mannitol, trehalose, raffinose, stachyose, xylitol, starch, verbascose, or any combination thereof. In some embodiments, the carbohydrate comprises sucrose. In some embodiments, sucrose is present at a mass concentration of solution between about 5 to 10% w/v of the pharmaceutical composition. In some embodiments, sucrose is present at a mass concentration of solution of about 8% w/v of the pharmaceutical composition. In some embodiments, the pharmaceutical composition further comprises a salt, wherein the salt is present at a concentration of about 1 mM to about 100 mM. In some embodiments, the salt comprises sodium, potassium, calcium, or ammonium. In some embodiments, the salt comprises sodium and wherein the sodium is present at a concentration of about 3 mM to about 10 mM. In some embodiments, the salt comprises sodium and wherein the sodium is present at a concentration of about 7 nM. In some embodiments, the pharmaceutical composition further comprises a buffering agent. In some embodiments, the pharmaceutical composition has a pH less than 7.5. In some embodiments, the pharmaceutical composition has a pH greater than 4.5. In some embodiments, the pharmaceutical composition has a pH of about 4.5 to about 7.5. In some embodiments, the pharmaceutical composition has a pH of about 5.5 to about 6.5. In some embodiments, the pharmaceutical composition has a pH of about 6.0. In some embodiments, the buffering agent comprises sucrose octa-sulfate, ammonium carbonate, ammonium phosphate, boric acid, sodium citrate, potassium citrate, lactic acid, 3-(N-morpholino)propanesulfonic acid (MOPS), 2-(N-morpholino)ethanesulfonic acid (MES), hydroxymethylaminomethane (Tris), calcium carbonate, calcium phosphate, or any combination thereof. In some embodiments, the buffering agent comprises sodium citrate at a concentration of about 3 mM to about 10 mM. In some embodiments, the buffering agent comprises sodium citrate at a pH of about 5.5 to about 6.5. In some embodiments, the buffering agent comprises sodium citrate at a concentration of about 7 mM. In some embodiments, the buffering agent comprises sodium citrate at a concentration of about 7 mM and a pH of about 6.0.

Described here are methods of treating a subject having myotonic muscular dystrophy, the methods comprising administering an effective amount of a fusion protein comprising the amino acid sequence of SEQ ID NO: 1 to the subject, wherein the subject has received a physician diagnosis of myotonic dystrophy type 1 (DM1) or myotonic dystrophy type 2 (DM2). In some aspects, the methods of treating a subject having myotonic muscular dystrophy comprise administering an effective amount of a fusion protein comprising the amino acid sequence of SEQ ID NO: 1 to the subject, wherein the subject has received a physician diagnosis of myotonic dystrophy type 1 (DM1). In some aspects, the methods of treating a subject having myotonic muscular dystrophy comprise administering an effective amount of a fusion protein comprising the amino acid sequence of SEQ ID NO: 1 to the subject, wherein the subject has received a physician diagnosis of myotonic dystrophy type 2 (DM2). In some embodiments, the administering comprises subcutaneous administration. In some embodiments, the administering comprises administration according to a dosage regime. In some embodiments, the dosage regime comprises once daily, once every other day, twice weekly, once weekly, once every ten days, once every two weeks, once every three weeks, once every month, once every two months, once every three months, once every four months, or once every six months administration of the fusion protein to the subject. In some embodiments, a pharmaceutical composition described herein is administered once daily. In some embodiments, a pharmaceutical composition described herein is administered once every other day. In some embodiments, a pharmaceutical composition described herein is administered twice weekly. In some embodiments, a pharmaceutical composition described herein is administered weekly. In some embodiments, a pharmaceutical composition described herein is administered every other week. In some embodiments, the effective amount of the fusion protein is less than about 0.5, 0.75, 1, 1.25, 1.5, 1.75, 2.0, 2.25, 2.5, 2.75, 3, 3.5, 4, 4.5, or 5 mg/kg of body weight of the subject. In some embodiments, the effective amount of the fusion protein is greater than about 0.01, 0.02, 0.05, 0.1, 0.2, 0.3, 0.4, 0.5, 0.6, 0.7, 0.75, 0.8, 0.9, 1, 1.1, 1.25, 1.35, 1.5, 1.65, 1.75, 1.85, 2.0, 2.25, 2.5, 2.75, or 3 mg/kg of body weight of the subject. In some embodiments, the effective amount of the fusion protein is between about 0.5 to about 4 mg/kg of body weight of the subject. In some embodiments, the effective amount of the fusion protein is between about 1 to about 3 mg/kg of body weight of the subject. In some embodiments, the effective amount of the fusion protein administered to the subject is adjusted to achieve a peak serum concentration (Cₘₐₓ) following subcutaneous administration of less than or equal to about 50, 40, 30, 25, 20, 17.5, 15, 12.5, 10, 7.5, 5 or 3 μg/mL. In some embodiments, the effective amount of the fusion protein administered to the subject is adjusted to achieve a peak serum concentration (Cₘₐₓ) following subcutaneous administration of less than or equal to 15 μg/mL. In some embodiments, the effective amount of the fusion protein administered to the subject is adjusted to achieve a peak serum concentration (Cₘₐₓ) following subcutaneous administration of greater than or equal to about 1, 2, 3, 4, 5, 6, 7, 8, 9, 10, 11, 12, 13, 14, 15, 16, 17, 18, 19, 20, 22.5, 25, 27.5, or 30 μg/mL. In some embodiments, the effective amount of the fusion protein administered to the subject is adjusted to achieve a peak serum concentration (Cₘₐₓ) following subcutaneous administration of between about 5 to about 25 μg/mL. In some embodiments, the subject receiving a physician diagnosis of DM1 has received genetic confirmation of DMPK repeat expansion. In some embodiments, the DMPK repeat expansion comprises greater than 100 CTG repeats in the DMPK gene. In some embodiments, the fusion protein agonistically promotes ERK1, ERK2, or ERK1 and ERK2 phosphorylation following the subcutaneous administration to the subject. In some embodiments, promotion of ERK1, ERK2, or ERK1 and ERK2 phosphorylation activates ERK1/2 downstream signaling following the subcutaneous administration to the subject. In some embodiments, the fusion protein agonistically promotes mitogen-activated protein kinase kinase (MEK) pathway activation following the subcutaneous administration to the subject. In some embodiments, the fusion protein agonistically activates PI3K signaling following the subcutaneous administration to the subject. In some embodiments, the fusion protein agonistically activates AKT signaling following the subcutaneous administration to the subject. In some embodiments, the activation of PI3K signaling agonistically activates AKT signaling following the subcutaneous administration to the subject. In some embodiments, the administering promotes muscle cell differentiation in the subject. In some embodiments, the administering promotes muscle cell survival in the subject. In some embodiments, the promotion of muscle cell survival in the subject leads to a lower rate of muscle cell degeneration compared to a subject having myotonic muscular dystrophy that has not been administered the fusion protein. In some embodiments, the administering improves muscle strength in the subject. In some embodiments, the administering reduces muscle wasting in the subject. In some embodiments, the administering comprises subcutaneous injection in the subject. In some embodiments, the pharmaceutical composition is administered with a syringe that contains 0.02 mL graduations. In some embodiments, the subject is at least 11 years old. In some embodiments, wherein dosage of the fusion protein administered to the subject per administration is about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3 mL of a pharmaceutical composition described herein. In some embodiments, dosage of the fusion protein administered to the subject is determined based on actual body of the subject at initiation of therapy. In some embodiments, the subject possesses an ability to walk independently for at least 10 meters at a time of screening prior to initiation of administration of the fusion protein. In some embodiments, the subject is assessed for the ability to walk independently with or without the use of orthoses or ankle braces.

Described here are methods of treating a subject having myotonic muscular dystrophy, comprising administering to the subject a pharmaceutical composition comprising a fusion protein comprising the amino acid sequence of SEQ ID NO: 1. In some aspects, the pharmaceutical composition is administered to the subject at a dose of at least about 5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 210, 225, 240, 250, 260, 275, 290, 300, 325, 350, 375, 400, 425, 450 mg of the fusion protein. In some aspects, the pharmaceutical composition is administered to the subject at a dose of at most about 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 210, 225, 240, 250, 260, 275, 290, 300, 325, 350, 375, 400, 425, 450, or 500 mg of the fusion protein. In some aspects, the pharmaceutical composition is administered to the subject at a dose of at most about 5 mg to 500 mg of the fusion protein. In some embodiments, the pharmaceutical composition is administered subcutaneously. In some embodiments, the subject is administered a pharmaceutical composition described herein according to the schedule of a dosage regime. In some embodiments, the dosage regime comprises once daily, once every other day, twice weekly, once weekly, once every ten days, once every two weeks, once every three weeks, once every month, once every two months, once every three months, once every four months, or once every six months administration of the fusion protein to the subject. In some embodiments, a pharmaceutical composition described herein is administered once daily. In some embodiments, a pharmaceutical composition described herein is administered once every other day. In some embodiments, a pharmaceutical composition described herein is administered twice weekly. In some embodiments, a pharmaceutical composition described herein is administered weekly. In some embodiments, a pharmaceutical composition described herein is administered every other week. In some embodiments, the effective amount of the fusion protein is less than about 0.5, 0.75, 1, 1.25, 1.5, 1.75, 2.0, 2.25, 2.5, 2.75, 3, 3.5, 4, 4.5, or 5 mg/kg of body weight of the subject. In some embodiments, the effective amount of the fusion protein is greater than about 0.01, 0.02, 0.05, 0.1, 0.2, 0.3, 0.4, 0.5, 0.6, 0.7, 0.75, 0.8, 0.9, 1, 1.1, 1.25, 1.35, 1.5, 1.65, 1.75, 1.85, 2.0, 2.25, 2.5, 2.75, or 3 mg/kg of body weight of the subject. In some embodiments, the effective amount of the fusion protein is between about 0.5 to about 4 mg/kg of body weight of the subject. In some embodiments, the effective amount of the fusion protein is between about 1 to about 3 mg/kg of body weight of the subject. In some embodiments, the effective amount of the fusion protein administered to the subject is adjusted to achieve a peak serum concentration (Cₘₐₓ) following subcutaneous administration of less than or equal to about 50, 40, 30, 25, 20, 17.5, 15, 12.5, 10, 7.5, 5 or 3 μg/mL. In some embodiments, the effective amount of the fusion protein administered to the subject is adjusted to achieve a peak serum concentration (Cₘₐₓ) following subcutaneous administration of less than or equal to 15 μg/mL. In some embodiments, the effective amount of the fusion protein administered to the subject is adjusted to achieve a peak serum concentration (Cₘₐₓ) following subcutaneous administration of greater than or equal to about 1, 2, 3, 4, 5, 6, 7, 8, 9, 10, 11, 12, 13, 14, 15, 16, 17, 18, 19, 20, 22.5, 25, 27.5, or 30 μg/mL. In some embodiments, the effective amount of the fusion protein administered to the subject is adjusted to achieve a peak serum concentration (Cₘₐₓ) following subcutaneous administration of between about 5 to about 25 μg/mL. In some embodiments, the subject has received a physician diagnosis of myotonic dystrophy type 1 (DM1) or myotonic dystrophy type 2 (DM2). In some embodiments, the subject has received a physician diagnosis of myotonic dystrophy type 1 (DM1). In some embodiments, the subject has received a physician diagnosis of myotonic dystrophy type 2 (DM2). In some embodiments, the subject receiving a physician diagnosis of DM1 has received genetic confirmation of DMPK repeat expansion. In some embodiments, the DMPK repeat expansion comprises greater than 100 CTG repeats in the DMPK gene. In some embodiments, the subject possesses an ability to walk independently for at least 10 meters at a time of screening prior to initiation of administration of the fusion protein. In some embodiments, the subject is assessed for the ability to walk independently with or without the use of orthoses or ankle braces.

In an aspect described herein are kits comprising pharmaceutical compositions described herein and instructions for use. In some embodiments, the instructions for use designate myotonic muscular dystrophy as an indication in a subject in need of treatment. In some embodiments, the instructions for use designate adult-onset myotonic dystrophy type 1 (adult-onset DM1) as an indication in a subject in need of treatment. In some embodiments, the kit further comprises a drug delivery device for administering the pharmaceutical composition to a subject. In some embodiments, the drug delivery device is a pre-filled syringe. In some embodiments, the pre-filled syringe has a set volume of about 0.4 mL, about 0.8 mL, about 1.0 mL, about 1.2 mL, or about 1.6 mL. In some embodiments, the pre-filled syringe has a set volume of about 0.4 mL, about 0.8 mL, about 1.0 mL, about 1.2 mL, about 1.6 mL. In some embodiments, the pre-filled syringe is configured to deliver a dosage of about 0.4 mg/mL, about 0.5 mg/mL, about 1.0 mg/mL, about 1.5 mg/mL, or about 2.0 mg/mL. In some embodiments, the pre-filled syringe contains a 100 mg/mL formulation concentration of the pharmaceutical composition.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1C depict induction of human DM1 protein kinase (DMPK) transgene expression in a mouse model of Myotonic Dystrophy Type 1 (DM1) and results of alternative splicing of Chloride channel, voltage-sensitive 1 (Clcn1) transcripts. FIG. 1A depicts a graph of relative hDMPK transgene expression following induction in panCUG₉₆₀/R26-M2rtTA (CUG₉₆₀) male mice compared to R26-M2rtTA+/−(rtTA(+/−)) control male mice. FIG. 1B depicts a graph of relative hDMPK transgene expression following induction in CUG₉₆₀ female mice compared to rtTA(+/−) control female mice. FIG. 1C shows induction of alternative splicing of Clcn1 mRNA transcripts in heart and thymus of CUG₉₆₀ mice. The 100 bp fragment is a Wild-type (Wt) splice variant and the 175 bp fragment is an alternative splice variant. (Abbreviations—GC: gastrocnemius; Dia: diaphragm; Br: brachioradialis)

FIG. 2A-FIG. 2D depict extent of muscle atrophy and grip strength reduction in males in a DM1 mouse model. FIG. 2A depicts significant reduction in bothlimb force in male CUG₉₆₀ mice compared to control rtTA+/− males. FIG. 2B depicts significant reduction in specific bothlimb force in male CUG₉₆₀ mice compared to control rtTA+/− males. FIG. 2C depicts significant reduction in forelimb force in male CUG₉₆₀ mice compared to control rtTA+/− males. FIG. 2D depicts significant reduction in specific forelimb force in male CUG₉₆₀ mice compared to control rtTA+/− males.

FIG. 3A-FIG. 3D depict extent of muscle atrophy and grip strength reduction in females in a DM1 mouse model. FIG. 3A depicts significant reduction in bothlimb force in female CUG₉₆₀ mice compared to control rtTA+/− females. FIG. 3B depicts reduction approaching significance in specific bothlimb force in female CUG₉₆₀ mice compared to control rtTA+/− females. FIG. 3C depicts significant reduction in forelimb force in female CUG₉₆₀ mice compared to control rtTA+/− females. FIG. 3D depicts significant reduction in specific forelimb force in female CUG₉₆₀ mice compared to control rtTA+/− females.

FIG. 4A-FIG. 4B depict immunofluorescent localization of RNA foci and MBNL1 in CUG₉₆₀ mice compared to control rtTA+/− mice. FIG. 4A shows induced RNA foci visualized with RNA-fluorescence in situ hybridization (FISH) and co-localization with MBNL1 in male CUG₉₆₀ mice. Panels on the right depict RNA foci-labeling merged with MBNL1 staining plus DAPI for visualization of the nucleus. FIG. 4B shows induced RNA foci visualized with RNA-FISH and co-localization with MBNL1 in female CUG₉₆₀ mice. Panels on the right depict RNA foci-labeling merged with MBNL1 staining plus DAPI for visualization of the nucleus.

FIG. 5A-FIG. 5B depict extent of muscle atrophy in a DM1 mouse model as measured by tibialis anterior (TA) muscle fiber cross-sectional area (CSA). FIG. 5A depicts a decrease in TA muscle fiber CSA in male CUG₉₆₀ mice compared to control rtTA+/− males. FIG. 5B depicts a decrease in TA muscle fiber CSA in female CUG₉₆₀ mice compared to control rtTA+/− females.

FIG. 6 depicts a graph of results and immunofluorescent staining images of muscle tissue sections demonstrating that HSA-IGF2-R61A treatment of human myoblasts leads to enhanced myogenesis via concentration-dependent human myoblast differentiation as measured by immunostaining for embryonic myosin heavy chain (% eMyHC). Proliferation of myoblasts was measured by Edu assay, differentiation of myoblasts was measured by eMyHC immunostaining, and nuclei were visualized via Hoechst staining.

FIG. 7 depicts administration of HSA-IGF2-R61A protected muscle atrophy and increased grip strength in male CUG₉₆₀ mice. PanCUG₉₆₀ male mice were treated Q.O.D with either 12 mg/kg body weight HSA-IGF2-R61A or PBS via s.c. injection. 3-week grip strength is graphed showing significant protection of bothlimb force, specific bothlimb force, forelimb force, and specific forelimb force. Grip strength significantly increased over time of treatment and specific grip strength was maintained at a significant level of difference compared to vehicle-treated male mice.

FIG. 8 depicts administration of HSA-IGF2-R61A protected muscle atrophy and increased grip strength in female CUG₉₆₀ mice. PanCUG₉₆₀ female mice were treated Q.O.D with either 12 mg/kg body weight HSA-IGF2-R61A or PBS via s.c. injection. 3-week grip strength is graphed demonstrating effects on bothlimb force, specific bothlimb force, forelimb force (approaching significant difference) and specific forelimb force (approaching significant difference). Grip strength significantly increased over time of treatment and specific grip strength was maintained relative to the observed decrease in vehicle-treated female mice.

FIG. 9 depicts the effects of administration of HSA-IGF2-R61A on grip strength in CUG₉₆₀ mice. A chart shows bothlimb grip strength (BL GST), specific BL GST, forelimb grip strength (FL GST), and specific FL GST preserved in male and female CUG₉₆₀ mice following Q.O.D administration of either 12 mg/kg body weight HSA-IGF2-R61A or PBS via s.c. injection. Over a 3 week treatment course, forelimb grip strength was preserved and significantly improved at 3 weeks.

FIG. 10 depicts the effects of administration of HSA-IGF2-R61A on TA cross-sectional area (CSA) in male CUG₉₆₀ mice. PanCUG₉₆₀ male mice were treated Q.O.D with either 12 mg/kg body weight HSA-IGF2-R61A or PBS via s.c. injection. Representative sections of muscle tissue stained for laminin and Hoechst used in the TA CSA calculations are shown. HSA-IGF2-R61A treatment significantly increased TA CSA in males.

FIG. 11 depicts the effects of administration of HSA-IGF2-R61A on TA cross-sectional area (CSA) in female CUG₉₆₀ mice. PanCUG₉₆₀ female mice were treated Q.O.D with either 12 mg/kg body weight HSA-IGF2-R61A or PBS via s.c. injection. Representative sections of muscle tissue stained for laminin and Hoechst used in the TA CSA calculations are shown. HSA-IGF2-R61A treatment significantly increased TA CSA in females.

FIG. 12A-FIG. 12B show graphs demonstrating pharmacokinetic (PK) profiles for HSA-IGF2-R61A following administration in mice. FIG. 12A shows the PK profile following s.c. injection with 12 mg/kg of body weight of HSA-IGF2-R61A in adult FVB/NJ mice and analyzed using a 1-compartment 1^{st} order model. FIG. 12B shows the PK profile following intravenous injection with 161 μg of HSA-IGF2-R61A in adult hFcRn-Tg MSA (−/−) mice and analyzed using a non-compartment model. Half-life was calculated at >136.5 hours.

FIG. 13A-FIG. 13B depict HSA-IGF2-R61A binding in a concentration-dependent manner in primary muscle cells. FIG. 13A is a graph demonstrating concentration-dependent binding of HSA-IGF2-R61A in rat primary muscle cells. FIG. 13B is a graph demonstrating concentration-dependent binding of HSA-IGF2-R61A in dog primary muscle cells.

FIG. 14 shows a graph depicting HSA-IGF2-R61A binding in a concentration-dependent manner in human primary muscle cells.

FIG. 15 shows a graph depicting a PK profile for HSA-IGF2-R61A following two separate s.c. administrations in the rat. Dose 1 (D1) was administered at time zero and Dose 2 (D2) was administered 96 hours after D1. A 1-compartment model closely fit the data following D1 allowing PK profile predictions following the administration of D2.

FIG. 16A-FIG. 16B depict PK parameters for HSA-IGF2-R61A following s.c. administration in the rat. FIG. 16A is a graph showing a calculation of maximum concentration (Cₘₐₓ) according to dosages tested of HSA-IGF2-R61A (in mg/kg of body weight). FIG. 16B is a graph showing a calculation of area under the curve up to the last quantifiable time point (AUC_{LAST}) according to dosages tested of HSA-IGF2-R61A (in mg/kg of body weight).

FIG. 17A-FIG. 17C depicts graphs showing muscle mass and metabolism improvement in DM1 mice (male CUG₉₆₀) at 3 weeks following s.c. treatment with HSA-IGF2-R61A. FIG. 17A shows a graph of body weight with HSA-IGF2-R61A or vehicle treatment indicating no significant overall change. Muscle weight (measured in gastrocnemius muscle) showed a significant increase in FIG. 17B and blood glucose change from baseline showed a significant decrease in FIG. 17C.

FIG. 18 depicts graphs and immunofluorescent staining on sections of soleus muscle tissue showing restoration of muscle composition in DM1 mice (male CUG₉₆₀) at 3 weeks following s.c. treatment with HSA-IGF2-R61A. Type2A fibers were significantly reduced and Type2B fibers were significantly increased in treated animals.

FIG. 19 depicts results from treatment with HSA-IGF2-R61A promoting human myogenesis in primary dystrophic muscle precursors by activating myogenic transcription factors. MYOG, CKM, and MYH3 expression are each elevated in human DM1 muscle precursors cells derived from 19-50 year-old male/female DM1 patients following treatment with HSA-IGF2-R61A. DMPK CTG repeats were evaluated and determined to range between 202-394 in the human DM1 muscle cells. Treatment with HSA-IGF2-R61A enhanced muscle degeneration.

FIG. 20 depicts results from treatment with HSA-IGF2-R61A improving human myogenesis in primary dystrophic muscle precursors. Treatment with HSA-IGF2-R61A significantly increased muscle precursor survival and regeneration in human DM1 muscle precursors cells derived from 19-50 year-old male/female DM1 patients.

FIG. 21 depicts a summary of reactome pathways modulated by HSA-IGF2-R61A treatment in cultured muscle cells derived from a 19 year old DM1 patient as assayed using RNAseq analysis on differentiating primary DM1 human myoblasts.

FIG. 22A-FIG. 22B show graphs of the Cmax and area under the curve (AUC) of the drug HSA-IGF2-R61A (combined sex) when administered at 6, 20, 60, and 120 mg/kg/dose once every 3 days for 18 days by SC injection under FED conditions. Cmax is graphed in FIG. 22A. AUC is graphed in FIG. 22B.

FIG. 23 shows a graph of Tmax after administration of various dosages of HSA-IGF2-R61A.

FIG. 24A-FIG. 24B show graphs of serum concentration of HSA-IGF2-R61A following IV administration (in FIG. 24A) or subcutaneous administration (in FIG. 24B) in dogs to demonstrate bioavailability.

FIG. 25A-FIG. 25B show graphs of serum IGF1 concentrations at 72 hr or 600 hr following IV administration (in FIG. 25A) or s.c. administration (in FIG. 25B) of HSA-IGF2-R61A.

### DETAILED DESCRIPTION

In certain aspects disclosed herein is a pharmaceutical composition comprising a fusion protein comprising Insulin like growth factor 2 (IGF2), or a functional fragment thereof, fused to a serum half-life extending moiety. In some aspects, the pharmaceutical composition comprises a fusion protein comprising human IGF2, or a functional fragment thereof, fused to a human serum albumin protein formulated with a stabilizer. In some aspects, the pharmaceutical composition comprises a fusion protein comprising human IGF2, or a functional fragment thereof, fused to a human serum albumin protein formulated with a surfactant. In some aspects, the pharmaceutical composition comprises a fusion protein comprising human IGF2, or a functional fragment thereof, fused to a human serum albumin protein formulated with a salt. In some aspects, the pharmaceutical composition comprises a fusion protein comprising human IGF2, or a functional fragment thereof, fused to a human serum albumin protein formulated with a buffer. In some aspects, the pharmaceutical composition comprises a fusion protein comprising human IGF2, or a functional fragment thereof, fused to a human serum albumin protein formulated with a buffer. In some aspects, the pharmaceutical composition comprises a fusion protein comprising human IGF2, or a functional fragment thereof, fused to a human serum albumin protein formulated as a buffered solution. In some aspects, the pharmaceutical composition comprises a fusion protein comprising human IGF2, or a functional fragment thereof, fused to a human serum albumin protein formulated for subcutaneous administration. In some aspects, the pharmaceutical composition comprises a fusion protein comprising human IGF2, or a functional fragment thereof, fused to a human serum albumin protein formulated to deliver a therapeutically effective dose following subcutaneous administration. In some aspects, the pharmaceutical composition comprises a fusion protein comprising human IGF2, or a functional fragment thereof, fused to a human serum albumin protein formulated in a dosage form of an injectable liquid having a volume to deliver a therapeutically effective dose by subcutaneous administration. In some aspects, the pharmaceutical composition comprises a fusion protein comprising the amino acid sequence of SEQ ID NO: 1, wherein the composition is formulated for subcutaneous administration. In some aspects, the pharmaceutical composition comprises a fusion protein comprising the amino acid sequence of SEQ ID NO: 2, wherein the composition is formulated for subcutaneous administration. In some aspects, the pharmaceutical composition comprises a fusion protein comprising the amino acid sequence of SEQ ID NO: 3, wherein the composition is formulated for subcutaneous administration. In some aspects, the pharmaceutical composition comprises a fusion protein comprising the amino acid sequence of SEQ ID NO: 4, wherein the composition is formulated for subcutaneous administration.

In certain aspects disclosed herein are methods for use of pharmaceutical compositions described herein for the treatment of muscle disorders. In some aspects disclosed herein are methods of treating a subject having myotonic dystrophy comprising administering to the subject a pharmaceutical composition described herein. In some aspects disclosed herein are methods of treating a subject having Myotonic Dystrophy Type 1 (DM1) comprising administering to the subject a pharmaceutical composition described herein. In some aspects disclosed herein are methods of treating a subject having Myotonic Dystrophy Type 1 (DM1) comprising administering to the subject a pharmaceutical composition described herein, wherein the subject has adult-onset DM1 (classical DM1). In some aspects disclosed herein are methods of treating a subject having Myotonic Dystrophy Type 1 (DM1) comprising administering to the subject a pharmaceutical composition described herein, wherein the subject has late-onset DM1. In some aspects disclosed herein are methods of treating a subject having Myotonic Dystrophy Type 1 (DM1) comprising administering to the subject a pharmaceutical composition described herein, wherein the subject has juvenile DM1. In some aspects disclosed herein are methods of treating a subject having Myotonic Dystrophy Type 1 (DM1) comprising administering to the subject a pharmaceutical composition described herein, wherein the subject has congenital DM1. In some aspects disclosed herein are methods of treating a subject having Myotonic Dystrophy Type 2 (DM2) comprising administering to the subject a pharmaceutical composition described herein. In some aspects disclosed herein are methods of treating a subject having sarcopenia comprising administering to the subject a pharmaceutical composition described herein. In some aspects disclosed herein are methods of treating a subject having glucocorticoid-induced muscle atrophy comprising administering to the subject a pharmaceutical composition described herein. In some aspects disclosed herein are methods of treating a subject having a muscular dystrophy comprising administering to the subject a pharmaceutical composition described herein. In some aspects disclosed herein are methods of treating a subject having an aging disorder comprising administering to the subject a pharmaceutical composition described herein. In some aspects disclosed herein are methods of treating a subject having a myotonic dystrophy comprising administering to the subject a pharmaceutical composition described herein, wherein the subject has received a physician diagnosis of myotonic dystrophy type 1 (DM1) or myotonic dystrophy type 2 (DM2). In some aspects disclosed herein are methods of treating a subject having a myotonic dystrophy comprising administering to the subject a pharmaceutical composition described herein, wherein the subject has received a physician diagnosis of myotonic dystrophy type 1 (DM1). In some aspects disclosed herein are methods of treating a subject having a myotonic dystrophy comprising administering to the subject a pharmaceutical composition described herein, wherein the subject has received a physician diagnosis of myotonic dystrophy type 2 (DM2).

### Pharmaceutical Compositions

In some aspects disclosed herein are pharmaceutical compositions comprising a fusion protein comprising the amino acid sequence of any one of SEQ ID NO: 1-4 and a stabilizer, wherein the stabilizer is present at a mass concentration of solution of about 0.5% to 15% weight/volume (w/v) of the pharmaceutical composition. In some aspects disclosed herein are pharmaceutical compositions comprising a fusion protein comprising the amino acid sequence of SEQ ID NO: 1 and a stabilizer, wherein the stabilizer is present at a mass concentration of solution of about 0.5% to 15% weight/volume (w/v) of the pharmaceutical composition. In some aspects disclosed herein are pharmaceutical compositions comprising a fusion protein comprising the amino acid sequence of SEQ ID NO: 1 and a stabilizer, wherein the stabilizer is present at a mass concentration of solution of at least about 5% w/v of the pharmaceutical composition. In some aspects disclosed herein are pharmaceutical compositions comprising a fusion protein comprising the amino acid sequence of SEQ ID NO: 1 and a stabilizer, wherein the stabilizer is present at a mass concentration of solution of less than about 10% w/v of the pharmaceutical composition. In some embodiments, the stabilizer is present at a mass concentration of solution about 0.5%, 1%, 1.5%, 2%, 2.5%, 3%, 3.5%, 4%, 4.5%, 5%, 5.5% 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15% w/v of the pharmaceutical composition. In some embodiments, the stabilizer is present at a mass concentration of solution between about 5 to 10% w/v of the pharmaceutical composition. In some embodiments, the stabilizer is present at a mass concentration of solution about 8% w/v of the pharmaceutical composition. In some embodiments, the stabilizer is a carbohydrate. In some embodiments, the carbohydrate comprises sucrose, mannitol, trehalose, raffinose, stachyose, xylitol, starch, verbascose, or any combination thereof. In some embodiments, the carbohydrate comprises sucrose. In some embodiments, the carbohydrate consists of sucrose. In some embodiments, sucrose is present at a mass concentration of solution between about 5 to 10% w/v of the pharmaceutical composition. In some embodiments, sucrose is present at a mass concentration of solution of about 8% w/v of the pharmaceutical composition. In some embodiments, sucrose is present at a concentration of about 60.0 to about 100.0 mg/mL. In some embodiments, sucrose is present at a concentration of about 70.0 to about 90.0 mg/mL. In some embodiments, sucrose is present at a concentration of about 80.0 mg/mL. In some embodiments, the pharmaceutical composition further comprises a surfactant. In some embodiments, the pharmaceutical composition further comprises a buffer. In some embodiments, the pharmaceutical composition further comprises a salt. In some embodiments, the pharmaceutical composition further comprises a buffering reagent.

In some aspects disclosed herein are pharmaceutical compositions comprising a fusion protein comprising the amino acid sequence of any one of SEQ ID NO: 1-4 and a surfactant, wherein the surfactant is present at a mass concentration of solution of about 0.001% to 1% w/v of the pharmaceutical composition. In some aspects disclosed herein are pharmaceutical compositions comprising a fusion protein comprising the amino acid sequence of SEQ ID NO: 1 and a surfactant, wherein the surfactant is present at a mass concentration of solution of about 0.001% to 1% w/v of the pharmaceutical composition. In some aspects disclosed herein are pharmaceutical compositions comprising a fusion protein comprising the amino acid sequence of SEQ ID NO: 1 and a surfactant, wherein the surfactant is present at a mass concentration of solution of at least about 0.01% w/v of the pharmaceutical composition. In some aspects disclosed herein are pharmaceutical compositions comprising a fusion protein comprising the amino acid sequence of SEQ ID NO: 1 and a surfactant, wherein the surfactant is present at a mass concentration of solution of less than about 1% w/v of the pharmaceutical composition. In some embodiments, the surfactant is present at a mass concentration of solution between about 0.05-0.20% w/v of the pharmaceutical composition. In some embodiments, the surfactant is present at a mass concentration of solution of about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% w/v of the pharmaceutical composition. In some embodiments, the surfactant is present at a mass concentration of solution between about 0.05-0.20% w/v of the pharmaceutical composition. In some embodiments, the surfactant is present at a mass concentration of solution between about 0.01% w/v of the pharmaceutical composition. In some embodiments, the surfactant is an ionic surfactant. In some embodiments, the surfactant is a nonionic surfactant. In some embodiments, the nonionic surfactant comprises a polysorbate or a poloxamer. In some embodiments, the nonionic surfactant comprises a polysorbate comprising Tween 20, Tween 40, Tween 60, Tween 80, Tween 65, Tween 80, or any combination thereof. In some embodiments, the nonionic surfactant comprises a poloxamer comprising poloxamer 124, poloxamer 188, poloxamer 237, poloxamer 338, poloxamer 407, or any combination thereof. In some embodiments, the nonionic surfactant comprises poloxamer 188. In some embodiments, the nonionic surfactant consists essentially of poloxamer 188. In some embodiments, the nonionic surfactant consists of poloxamer 188. In some embodiments, the nonionic surfactant comprises poloxamer 188 at a mass concentration of solution of about 0.1% w/v. In some embodiments, the pharmaceutical composition further comprises a stabilizer. In some embodiments, the pharmaceutical composition further comprises a buffer. In some embodiments, the pharmaceutical composition further comprises a salt. In some embodiments, the pharmaceutical composition further comprises a buffering reagent.

In some aspects disclosed herein are pharmaceutical compositions comprising a fusion protein comprising the amino acid sequence of any one of SEQ ID NO: 1-4 and a salt, wherein the salt is present at a concentration of about 1 mM to about 100 mM. In some aspects disclosed herein are pharmaceutical compositions comprising a fusion protein comprising the amino acid sequence of SEQ ID NO: 1 and a salt, wherein the salt is present at a concentration of about 1 mM to about 100 mM. In some embodiments, the salt comprises sodium, potassium, calcium, or ammonium. In some embodiments, the salt is present at a concentration about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the salt comprises sodium and wherein the sodium is present at a concentration of about 3 mM to about 10 mM. In some embodiments, the salt comprises sodium and wherein the sodium is present at a concentration of about 7 nM. In some embodiments, the pharmaceutical composition further comprises a surfactant. In some embodiments, the pharmaceutical composition further comprises a stabilizer. In some embodiments, the pharmaceutical composition further comprises a buffering agent.

In some aspects disclosed herein are pharmaceutical compositions comprising a fusion protein comprising the amino acid sequence of any one of SEQ ID NO: 1-4 and a buffer, wherein the buffer is present at a concentration of about 1 mM to about 100 mM. In some aspects disclosed herein are pharmaceutical compositions comprising a fusion protein comprising the amino acid sequence of SEQ ID NO: 1 and a buffer, wherein the buffer is present at a concentration of about 1 mM to about 100 mM. In some embodiments, the buffer comprises sodium, potassium, calcium, or ammonium. In some embodiments, the buffer is present at a concentration about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM. In some embodiments, the buffer comprises sodium and wherein the sodium is present at a concentration of about 3 mM to about 10 mM. In some embodiments, the buffer comprises sodium and wherein the sodium is present at a concentration of about 7 nM. In some embodiments, the pharmaceutical composition further comprises a surfactant. In some embodiments, the pharmaceutical composition further comprises a stabilizer. In some embodiments, the pharmaceutical composition further comprises an additional buffering agent. In some embodiments, the buffer is a salt. In some embodiments, the additional buffering agent is a salt.

In some aspects disclosed herein are pharmaceutical compositions comprising a fusion protein comprising the amino acid sequence of any one of SEQ ID NO: 1-4 and a buffering agent. In some aspects disclosed herein are pharmaceutical compositions comprising a fusion protein comprising the amino acid sequence of SEQ ID NO: 1 and a buffering agent. In some embodiments, the pharmaceutical composition has a pH less than 7.5. In some embodiments, the pharmaceutical composition has a pH greater than 4.5. In some embodiments, the pharmaceutical composition has a pH of about 4.5 to about 7.5. In some embodiments, the pharmaceutical composition has a pH of about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6. 8, 6.9, 7.0, 7.1, 7.2, 7.3, 7.4, or 7.5. In some embodiments, the pharmaceutical composition has a pH of about 5.5 to about 6.5. In some embodiments, the pharmaceutical composition has a pH of about 6.0. In some embodiments, the buffering agent comprises sucrose octa-sulfate, ammonium carbonate, ammonium phosphate, boric acid, sodium citrate, potassium citrate, lactic acid, 3-(N-morpholino)propanesulfonic acid (MOPS), 2-(N-morpholino)ethanesulfonic acid (MES), hydroxymethylaminomethane (Tris), calcium carbonate, calcium phosphate, or any combination thereof. In some embodiments, the buffering agent comprises sodium citrate at a concentration of about 3 mM to about 10 mM. In some embodiments, the buffering agent comprises tri-sodium citrate dihydrate at a concentration of about 1.5 to 2.0 mg/mL. In some embodiments, the buffering agent comprises tri-sodium citrate dihydrate at a concentration of about 1.6 to 1.85 mg/mL. In some embodiments, the buffering agent comprises tri-sodium citrate dihydrate at a concentration of about 1.74 mg/mL. In some embodiments., the buffering agent comprises citric acid monohydrate at a concentration of about 0.1 to about 0.35 mg/mL. In some embodiments, the buffering agent comprises citric acid monohydrate at a concentration of about 0.15 to about 0.30 mg/mL. In some embodiments, the buffering agent comprises citric acid monohydrate at a concentration of about 0.23 mg/mL. In some embodiments, the buffering agent comprises sodium citrate at a pH of about 5.5 to about 6.5. In some embodiments, the buffering agent comprises sodium citrate at a concentration of about 7 mM. In some embodiments, the buffering agent comprises sodium citrate at a concentration of about 7 mM and a pH of about 6.0. In some embodiments, the pharmaceutical composition further comprises a surfactant. In some embodiments, the pharmaceutical composition further comprises a stabilizer. In some embodiments, the pharmaceutical composition further comprises a salt. In some embodiments, the pharmaceutical composition further comprises a buffer. In some embodiments, the pharmaceutical composition further comprises an additional buffering agent.

### Formulations

In some aspects disclosed herein are pharmaceutical compositions comprising a fusion protein comprising the amino acid sequence of any one of SEQ ID NO: 1-4, wherein the pharmaceutical compositions are formulated for subcutaneous administration. In some aspects disclosed herein are pharmaceutical compositions comprising a fusion protein comprising the amino acid sequence of SEQ ID NO: 1, wherein the pharmaceutical compositions are formulated for subcutaneous administration. In some embodiments, the pharmaceutical composition is formulated wherein the fusion protein is in a dosage form of injectable liquid having a volume of about 0.1 mL to about 3 mL, or about 0.5 mL to about 2 mL, or about 0.1 mL to about 1 mL. In some embodiments, the pharmaceutical composition is formulated wherein the fusion protein is in a dosage form of injectable liquid having a volume of about 0.1 mL to about 3 mL. In some embodiments, the pharmaceutical composition is formulated wherein the fusion protein is in a dosage form of injectable liquid having a volume of about 0.5 mL to about 2 mL. In some embodiments, the pharmaceutical composition is formulated wherein the fusion protein is in a dosage form of injectable liquid having a volume of about 0.1 mL to about 1 mL. In some embodiments, the pharmaceutical composition is formulated wherein the fusion protein is in a dosage form of injectable liquid having a volume of about 0.1, 0.2, 0.25, 0.3, 0.4, 0.5, 0.6, 0.7, 0.75, 0.8, 0.9, 1.0, 1.1, 1.2, 1.25, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 mL. In some embodiments, the pharmaceutical composition is formulated wherein the fusion protein is in a dosage form of injectable liquid having a volume of about 0.4 mL. In some embodiments, the pharmaceutical composition is formulated wherein the fusion protein is in a dosage form of injectable liquid having a volume of about 0.8 mL. In some embodiments, the pharmaceutical composition is formulated wherein the fusion protein is in a dosage form of injectable liquid having a volume of about 1.0 mL. In some embodiments, the pharmaceutical composition is formulated wherein the fusion protein is in a dosage form of injectable liquid having a volume of about 1.1 mL. In some embodiments, the pharmaceutical composition is formulated wherein the fusion protein is in a dosage form of injectable liquid having a volume of about 1.2 mL. In some embodiments, the pharmaceutical composition is formulated wherein the fusion protein is in a dosage form of injectable liquid having a volume of about 1.6 mL. In some embodiments, the pharmaceutical composition is formulated wherein the fusion protein is present at a concentration of about 30 mg/mL to about 350 mg/mL. In some embodiments, the pharmaceutical composition is formulated wherein the fusion protein is present at a concentration of about 100 mg/mL to about 200 mg/mL. In some embodiments, the pharmaceutical composition is formulated wherein the fusion protein is present at a concentration of about 40 mg/mL to about 160 mg/mL. In some embodiments, the pharmaceutical composition is formulated wherein the fusion protein is present at a concentration of about 80 mg/mL to about 160 mg/mL. In some embodiments, the pharmaceutical composition is formulated wherein the fusion protein is present at a concentration of about 40 mg/mL. In some embodiments, the pharmaceutical composition is formulated wherein the fusion protein is present at a concentration of about 60 mg/mL. In some embodiments, the pharmaceutical composition is formulated wherein the fusion protein is present at a concentration of about 80 mg/mL. In some embodiments, the pharmaceutical composition is formulated wherein the fusion protein is present at a concentration of about 100 mg/mL. In some embodiments, the pharmaceutical composition is formulated wherein the fusion protein is present at a concentration of about 120 mg/mL. In some embodiments, the pharmaceutical composition is formulated wherein the fusion protein is present at a concentration of about 140 mg/mL. In some embodiments, the pharmaceutical composition is formulated wherein the fusion protein is present at a concentration of about 160 mg/mL. In some embodiments, the pharmaceutical composition is formulated wherein the fusion protein is present at a concentration of about 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195,200, 210, 220, 225, 230, 240, 250, 260, 270, 275, 280, 290, 300, 310, 320, 325, 330, 340, or 350 mg/mL. In some embodiments, a pre-filled syringe containing the pharmaceutical composition having a set volume between about 0.4-1.6 mL is configured to deliver a dosage of between about 0.5-2.0 mg/mL of HSA-IGF2-R61A to the subject. In some embodiments, a pre-filled syringe contains a set volume of about 0.4 mL, a 100 mg/mL of HSA-IGF2-R61A formulation concentration, and is configured to deliver a dosage of about 0.5 mg/kg to an 80 kg subject. In some embodiments, a pre-filled syringe contains a set volume of about 0.8 mL, a 100 mg/mL of HSA-IGF2-R61A formulation concentration, and is configured to deliver a dosage of about 1.0 mg/kg to an 80 kg subject. In some embodiments, a pre-filled syringe contains a set volume of about 1.2 mL, a 100 mg/mL of HSA-IGF2-R61A formulation concentration, and is configured to deliver a dosage of about 1.5 mg/kg to an 80 kg subject. In some embodiments, a pre-filled syringe contains a set volume of about 1.6 mL, a 100 mg/mL of HSA-IGF2-R61A formulation concentration, and is configured to deliver a dosage of about 2.0 mg/kg to an 80 kg subject.

In some aspects disclosed herein are pharmaceutical composition comprising a fusion protein comprising the amino acid sequence of SEQ ID NO: 1, a pharmaceutically acceptable excipient, carrier, or diluent, wherein the pharmaceutical composition is formulated for subcutaneous administration. The excipients used in a pharmaceutical composition can provide additional function to the fusion protein by making the fusion protein suitable for a particular route of administration (e.g., subcutaneous), increasing fusion protein stability, increasing penetration of a desired tissue (e.g., muscle or skin), increasing residence time at particular site, increasing solubility, enhancing the efficacy of the polypeptide, and/or reducing inflammatory reactions coincident with administration. Formulations suitable for subcutaneous injection can include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include ethanol, polyols (inositol, propyleneglycol, polyethylene-glycol, glycerol, cremophor and the like) and suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

### Myotonic Dystrophy

Myotonic dystrophies (DM) are autosomal dominantly inherited, multisystemic diseases. DM is classified into two distinct forms based on clinical and molecular characteristics:
DM type 1 (dystrophia myotonica type I; DM1; OMIM #160900, also known as Steinert's disease) is caused by unstable expansion of CTG trinucleotide repeats located in the 3′ untranslated region (UTR) of the DMPK gene and
DM type 2 (dystrophia myotonica type 2, DM2; OMIM #602668 also known as Ricker syndrome or proximal myotonic dystrophy [PROMM]) is caused by CCTG repeats in CCHC-type zinc finger nucleic acid binding protein (CNBP).

DM1 is more common and represents a more severe phenotype than DM2.

DM1 is a multisystem disorder that affects skeletal and smooth muscle as well as the eye, heart, endocrine system, and central nervous system. The clinical findings span a continuum from mild to severe and include myotonia, progressive, muscle weakness, early-onset cataracts, dysphagia, neuromuscular respiratory insufficiency, cardiac arrhythmias and conduction defects, endocrine disorders, cognitive, intellectual or behavioral impairment, sleep disorders and increased risk of developing neoplasias. In the most severe forms, life quality and expectancy are seriously compromised.

The current prevalence estimate of DM1 is between 5 and 20 per 100,000 individuals worldwide. A high prevalence is reported in certain areas with founder mutations (e.g., Quebec in Canada and the Basque Country in Spain). A recent newborn screening study was conducted in the United States identifying DMPK CTG expansion. CTG repeats >50 were identified with a prevalence of 4.76 per 10,000 births (prevalence of 47.6/100,000). Given the current US population of approximately 333 million individuals (2020 census), the prevalence of DM1 is approximately 159,000 individuals (Census.gov). Thus, DM1 meets the statutory definition of a rare disease (orphan disease) in the United States (e.g., a prevalence of <200,000 individuals).

As shown in Table 1, DM1 is generally categorized into five clinical phenotypes defined by the age of onset of symptoms.

TABLE 1
Five Phenotypes of DM1

| Phenotype | Symptom onset |
|---|---|
| Congenital | present at birth or noticed in |
| | the first month of life |
| Childhood/infantile | Day 31 until age 10 |
| Juvenile/early adult | 10 to 20 years of age |
| Adult-onset | 20 to 40 years of age |
| (“Classic”) | |
| Late-onset | Over 40 years of age |

### DM1 Etiology

DM1 is caused by unstable expansion of the cytosine thymine-guanine (CTG) trinucleotide repeat located in the 3′ untranslated region (UTR) of the dystrophia myotonica protein kinase (DMPK) gene, chromosome 19q13.3, encoding a serine/threonine protein kinase (DMPK) trinucleotide.

DM1 is transmitted across generations in an autosomal dominant fashion with incomplete penetrance, variable phenotypic expression and somatic mosaicism. Anticipation, i.e., increased number of CTG repeats in subsequent generations, is associated with increased severity of disease and is more marked with maternal transmission.

The disease mechanism involves a toxic gain-of-function of DMPK transcripts that carry expanded cytosine-uracil-guanine (CUG) repeats (CUGexp), which are transcribed into RNA forming ribonuclear foci comprised of auto complementary CUG hairpin structures that can bind proteins in myotonic dystrophy type 1-affected tissues, such as muscle, heart, and brain, among others. The bound proteins commonly include components of the spliceosome and mRNA processing, competitively inhibiting their normal functions on other genes' transcripts. Collectively, these effects cause misregulated alternative splicing or polyadenylation for certain mRNAs.

The toxic DMPK transcripts affect several other signaling cascades which play important roles in the pathogenesis of DM1. For example, the impairment of protein kinase B (AKT) signaling in DM1 increases autophagy, apoptosis, and ubiquitin-proteasome activity, which may also be affected in DM1 by AMP-activated protein kinase (AMPK) downregulation.

Different mechanisms have been proposed to contribute to the pathology in DM1 that include: (a) DMPK haploinsufficiency; (b) alteration of neighboring genes; and/or (c) RNA-induced toxicity. In some instances, patients are selected for treatment with HSA-IGF2-R61A following diagnosis of DMPK haploinsufficiency.

DMPK is normally expressed in muscle and haploinsufficiency has been proposed due to the detection of reduced DMPK transcripts and proteins, which is correlated with the CTG repeat length. Most recently, mRNA toxicity from the transcription of the CTGn repeats into cytosine-uracil-guanine (CUG)n has advanced as an important part of the etiology. The expanded CUG RNAs become arrested in the nucleus and form ribonuclear foci composed of hairpin structures that bind and sequester essential splicing proteins, e.g., Muscle-blind-like proteins (MBNL) and hyperphosphorylation of CUG triplet repeat, RNA binding protein 1 (CUGBP) myoblasts, skeletal muscle and heart tissues. This stressor in RNA-processing results in complex molecular changes dysregulating signaling in a few central pathways altering the function of polyadenylation, RNA stability, and translation initiation regulator CUGBP Elav-Like Family Member 1 (CELF1). Among these pathways, recent evidence highlights reduced Protein kinase B (AKT) signaling as the driver of key aspects of cellular dysregulation in DM1, such as apoptosis, autophagy and myogenesis. Reduced AKT signaling results in dysregulation of CELF1 both directly through mislocalized accumulation in the nucleus and indirectly through a hyperactive glycogen synthase kinase 3 beta (GSK30) preventing CELF1 degradation. In healthy cells, AKT activation represses GSK30 activity. Up-regulated expression of the glucocorticoid effector protein Forkhead box protein O1 (FoxO1), as well as proteins involved in the autophagy pathway regulated by FoxO1 further contribute to muscle atrophy in DM1 models. Collectively these alterations in molecular mechanisms underlie skeletal muscle loss via increased autophagy and apoptosis and decreased translational and myogenic processes. In subjects with DM1, RNA splicing failure and depletion can lead to increases in stress response and increased glucocorticoid receptor signaling. Changes in RNA splicing have been implicated in several aspects of DM1 disease manifestation, including myotonia, memory impairment, and insulin resistance. The insulin resistance has been attributed to a change in the splicing of the insulin receptor from the beta (IR-B) isoform, normally predominant in adult muscle, to the alpha isoform (IR-A), normally predominantly expressed in fetal contexts. The alpha isoform has lower affinity for insulin and thus this shift is postulated to be an important contributor to the emergent insulin resistance that impacts myogenesis, muscle function, and fatigue in DM1. Reduction of circulating IGFs can lead to reductions in PI3K/AKT signaling. Reductions in PI3K/AKT signaling in DM1 muscle cells can lead to impaired muscle differentiation and reduced muscle cell survival. In some instances in subjects with DM1, insulin receptor (IR) reverts to embryonic isoform (IR-A) which can further lead to increase insulin insensitivity and increased muscle fatigue. Compounding the shift in insulin receptor isoforms is a reduction in the circulating concentrations of components of the IGFs system in DM1. Together the dysregulation of both the ligand and receptor sides of the insulin and IGF system contribute to the reduced PI3K/AKT signaling implicated as the driver of key aspects of cellular dysregulation in DM1. DM1 molecular etiology can have one or more of several consequences leading to muscle cell degeneration that characterizes the disorder. In some instances, DMPK mRNA splicing failure and/or depletion lead to an increase in stress response and increased glucocorticoid receptor signaling. In some instances, reducing circulating IGFs as a consequence of DM1 molecular etiology produce reduced PI3K/AKT signaling. In some instances, PI3K/AKT signaling is reduces in DM1 muscle cells leading to impaired myoblast differentiation and/or impaired myoblast survival or myocyte survival. In some instances, the IR isotype reverts to an embryonic isoform (IR-A) which produces an increase in insulin insensitivity and an increase in muscle fatigue.

These pathogenic mechanisms affect various cellular processes involved in the muscular degeneration seen in DM1 patients, which are similar to the multisystem involvement induced by premature aging. Skeletal muscle is one of the most severely affected tissues in DM1 and may age prematurely, which mimics sarcopenia (age-related loss of muscle mass and function). Satellite cell dysfunction has been hypothesized to be the main contributor to muscle wasting in patients with DM1 and sarcopenia.

Furthermore, histopathological features observed in skeletal muscle biopsies include a marked increase in central nuclei, pyknotic nuclear clumps, ring fibers, irregular nuclei shape, and myofiber size variability. Other features include preferential atrophy of type 1 fibers.

As shown in Table 2, the number of CTG repeats within the DMPK gene is correlated with the various DM1 phenotypes, disease severity, and age of onset.

TABLE 2
Summary of DM1 Phenotypes, Clinical Symptoms, and CTG Repeat Range

| | | Repeat | Age of | |
|---|---|---|---|---|
| Phenotype | Most Prominent Clinical Symptoms | Range | Onset | Life Span |
| Late-onset | Cataracts, hypersomnia, myotonia | 100-600 | >40 | Normal |
| DM1 | | | years | |
| Adult-onset | Myotonia, cardiac arrhythmias, | 250-750 | 20-40 | Shortened |
| “classical | hypersomnia, gastrointestinal | | years | |
| DM1” | difficulties, muscle weakness and | | | |
| | wasting, cataracts, male | | | |
| | hypogonadism, insulin resistance, | | | |
| | cognitive challenges, left ventricular | | | |
| | dysfunctions | | | |
| Juvenile | Similar symptoms as adult DM1 but | 400-800 | 10-20 | Shortened |
| DM1 | more severe | | years | |
| Infantile | Similar symptoms as congenital DM1 | 500-1100 | 1 month- | Shortened |
| DM1 | but less severe | | 10 years | |
| Congenital | Developmental defects, hypotonia, | 750-1400 | Birth | At increased |
| DM1 | respiratory insufficiency, cardiac | | | risk of infant |
| | defects, severe cognitive challenges, | | | mortality or |
| | facial dysmorphism, dysphagia | | | shortened |

### Clinical Presentation of Adult-onset DM1

Adult-onset-DM1 is characterized by skeletal muscle dysfunctions, pronounced muscle atrophy particularly of proximal muscle groups, and delayed muscle relaxation (myotonia). Progressive muscle wasting erodes individuals' ability to walk, swallow, and manually manipulate objects and commonly culminates in cardiovascular illness-related premature death in the 6th decade. DM1 myopathy is also commonly accompanied by multisystem manifestations such as developmental disorders, personality and behavioral disturbances, affective symptoms, insulin resistance, alopecia, cognitive declines, infertility, skin tumors, cataracts, sleep disorders, and digestive tract issues.

Age of onset varies by patient; adult-onset occurs in adulthood with an overall life expectancy of 45-60 years. Due to diverse nature of the disease, there is usually a delay in accurate diagnosis which may be several years in some patients. Available literature reflects potential gender differences in the clinical presentation of DM1; males experience higher morbidity and mortality than females due to a higher prevalence of severe muscular disability with marked myotonia and muscle weakness, coupled with cardiac, respiratory and cognitive abnormalities. Females show a higher prevalence of cataracts, dysphagia, digestive tract dysfunction, incontinence, thyroid disorder, and obesity. Patients with adult-onset DM1 have a shortened life expectancy. Respiratory insufficiency complicated by pneumonia and cardiac arrhythmias are the most common causes of death.

As an overview of adult-onset DM1 clinical presentation, the following organ system deficiencies may manifest in DM1 patients as seen in Table 3A. Muscular and Extra-muscular Characteristics of Myotonic Dystrophy (DM) Type 1 are listed in Table 3B.

TABLE 3A
Overview of adult-onset DM1 clinical presentation

| Affected organ system | Deficiencies |
|---|---|
| Muscle | Weakness; wasting (atrophy); myotonia; pain |
| CNS | Altered cognitive function; intellectual impairment, |
| | behavioral and psychological disorders, excessive daytime |
| | sleepiness |
| Vision | Cataracts; retinal damage |
| Endocrine system | Diabetes; low thyroid hormone levels |
| Respiratory system | Breathing difficulties; aspiration; sleep apnea; high risk for |
| | developing pneumonia |
| Skin | Pilomatrixomas |
| Immune system | Hypogammalobulinemia |
| Male reproductive | Low testosterone levels; erectile dysfunction; testicular failure |
| system | and gonadal atrophy |
| Female reproductive | Weakened uterine muscle; pregnancy-related complications; |
| system | gynecological problems |
| Bones | Anomalies |
| Cardiovascular system | Heart condition abnormalities; arrhythmias; cardiomyopathy |
| Gastrointestinal | Swallowing difficulties; abdominal pain; irritable bowel |
| | syndrome; constipation/diarrhea; poor nutrition; weight loss; |
| | chronic infection |

TABLE 3B
Muscular and Extra-muscular Characteristics of Myotonic Dystrophy (DM) Type I

| Muscular Effects | | Extra-muscular Effects | |
|---|---|---|---|
| Muscle groups | Facial, neck, forearm, | Cataracts | Very common; almost universal late |
| commonly affected | hand intrinsic, foot | | in course |
| | dorsiflexor | | |
| Clinical myotonia | Common; often | Cardiac abnormalities | Conduction disturbances well |
| (grip and percussion) | pronounced | | recognized and common late in course |
| | | | Progressive cardiomyopathy also |
| | | | described |
| Muscle pain* | Yes | Cognitive impairment; | Intellectual disability is common with |
| | (Rarely presenting | personality | congenital DM I |
| | complaint) | disturbance | mild to moderate cognitive and |
| | | | personality defects in adult form |
| Respiratory muscle | Yes | Endocrine disturbance | Glucose intolerance: well recognized |
| involvement^{Δ} | | | and common late in course |
| | | | Hypogonadism: common and almost |
| | | | universal late in course |
| Dysphagia and | Yes | Gastrointestinal | Irritable bowel symptoms |
| dysarthria^{Δ} | | disorder | dysphagia |
| | | | gall stones |

Harper P S. Myotonic Dystrophy, 2nd ed, W B Saunders, London 1989.
*Pain is generally not correlated with myotonia.
^{Δ}Important contributors to pneumonia and respiratory failure.

### Skeletal Muscle Weakness in DM1

Skeletal muscle weakness is a characteristic feature of DM1. Weakness occurs most frequently in “distal” muscles, i.e., facial muscles, sternocleidomastoids, distal muscles of the forearm, hand intrinsic muscles (leading to compromised finger dexterity), and ankle dorsiflexors (causing bilateral foot drop). Less commonly, weakness occurs in the quadriceps, respiratory muscles, palatal and pharyngeal muscles, tongue, and extraocular muscles. Muscles of the pelvic girdle, the hamstrings, and ankle plantar flexors are relatively spared in most cases of DM1.

The facial appearance of patients with DM1 reflects longstanding muscle weakness and wasting: The face is long and narrow, and the palate is high arched. The cheeks are hollowed and the jaw sags. Ptosis and wasting of the sternocleidomastoid muscles are common in DM1.

The natural history of DM1 is that of gradual progression in weakness. The consequences are diminished mobility and increased rates of falls. As a precaution, patients decrease external activities, diminish social interaction and have a diminished quality of life.

In a study of 204 adult DM1 patients with confirmed genetic testing, the decline in muscle strength (measured by manual muscle testing, MMT) tended to be slow (0.2-0.3% per year; foot dorsiflexors (0.65% per year) and foot plantarflexors (0.5% per year) were the most affected). The rate of decline was similar for men and women. The rate of decline in handgrip force was 2.4% per year. Notably approximately 85% of the patients had limitations in mobility or walking.

In a study of 100 adults with late-onset or adult DM1 phenotypes, eight muscle groups were measured at baseline and 9 years later using standardized quantitative muscle testing. For the whole group, a mean loss of 24.5-−52.8% was observed over the 9-year period for all muscle groups, except for hip flexors which remained stable. Generally, men were stronger and showed a significant greater rate of decline of muscle strength than women. The highest strength loss was found in distal muscle groups (wrist extensors and ankle dorsiflexors), with a decrease over 50% of muscle strength.

In a series of 158 patients with DM1 who had assessment of finger flexor muscle strength using a hand-held dynamometer, strength diminished at a rate of 1.18 kgN/year for women and 1.61 kgN/year for men.

Consistent with observations related to progressive decline in strength in foot dorsiflexors and plantar flexors, gait is progressively impaired and postural stability declines in individuals with DM1. These changes are associated with an increase in the rate of stumbles and falls that is approximately 10 times more frequent than age-matched controls.

### Prevalence of Adult-Onset DM1

Adult-onset DM1 is the most common form of DM1; however, the available prevalence data does not distinguish subtypes of DM1. DM1 is an orphan disease and affects anywhere between 1,666.4-159,978 people in the United States (US) with a prevalence rate of 0.5-48/100,000 (range of 1,666.4-159,978 based on July 2022 estimated US population). While adult-onset DM1 is a distinctive form of DM1, incidence and prevalence data do not provide specific breakdown of patients who have been diagnosed with one of the five DM1 subtypes. Due to limitations in adult-onset DM1 reporting, the true prevalence of this DM1 subtype is expected to be lower than the highest US prevalence of 159,978 calculated from Johnson N E et al. Population-Based Prevalence of Myotonic Dystrophy Type 1 Using Genetic Analysis of Statewide Blood Screening Program. Neurology. 2021 Feb. 16; 96(7):e1045-e1053.

### Rationale for Use of HSA-IGF2-R61A Fusion Protein for the Treatment of Myotonic Dystrophy

Treatment comprising administering the fusion protein HSA-IGF2-R61A may improve muscle strength, endurance, mass, and glucose regulation, leading to reduced atrophy together with faster walk speeds and reduced fall rates, in adult-onset DM1 patients. In some instances of pharmaceutical compositions and methods of use described herein, the proposed HSA-IGF2-R61A indication is for the treatment of symptoms associated with adult-onset myotonic dystrophy type 1 (DM1). In some instances of methods described herein, HSA-IGF2-R61A treatment improves muscle strength, endurance, mass, and glucose regulation, leading to reduced muscle atrophy, greater mobility, faster walk speeds, reduced rates of falling, increased social participation, and/or overall improved quality of life in adult-onset DM1 patients.

HSA-IGF2-R61A activates PI3K/AKT/mammalian target of rapamycin (mTOR) signaling with sphingosine-1-phosphate (S1P) and bone morphogenetic protein (BMP) crosstalk to promote muscle differentiation, survival, and strength. AKT signaling is reduced in adult-onset DM1 muscle cells, driving deficits in myogenesis and muscle homeostasis. Activation of endogenous muscle stem cells by HSA-IGF2-R61A may restore and/or preserve muscle strength and function in patients with degenerative muscle diseases, a group of individuals whose declining muscle function leads to loss of ambulation, problems swallowing or breathing, and other disabilities. In some embodiments of methods described herein, HSA-IGF2-R61A activates endogenous muscle stem cells with the goals to restore and/or preserve muscle strength and function in patients with degenerative muscle diseases whose muscle function otherwise declines, leading to loss of ambulation, problems swallowing or breathing, and other disabilities.

HSA-IGF2-R61A maintains a higher affinity than IGF-1 for the embryonic IR-A, which is upregulated in adult-onset DM1 patients. Treatment for DM1 comprising administering HSA-IGF2-R61A to a patient having DM1 is expected to activate PI3K/AKT signaling, restore circulating IGF signaling (compensate for lower circulating IGF-1 via interaction with IGF receptors), and inhibit glucocorticoid receptor mediated-atrophy. In some embodiments of methods described herein, HSA-IGF2-R61A exhibits cellular effects in adult DM1 patients following administering of a pharmaceutical composition described herein. In some aspects, the pharmaceutical composition comprises HSA-IGF2-R61A. In some embodiments, one of more of the following cellular features of DM1 disease show improvement following the administration of the pharmaceutical composition comprising HSA-IGF2-R61A: inactive muscle stem cells, improper cell cycle exit during myogenesis, poor myocyte fusion, pathological increase of apoptosis, faulty CA²⁺ transport, pathological increase in autophagy, decreased insulin signaling, and decreased IF signaling. In some embodiments, one of more of the following cellular features of DM1 disease may show improvement following the administration of the pharmaceutical composition comprising HSA-IGF2-R61A: epigenetic dysregulation, RNA splicing defects, CUGBP overactivation, Muscle blind-like protein (MBNL) repression, and stalled replication forks and R-loops.

### Insulin-Like Growth Factors (IGF) and Insulin-Like Growth Factor Receptors

Insulin-like growth factors (IGF) are part of a complex system, the components of which act together to influence growth. The system consists of insulin, IGF-1, IGF-2, their cell surface receptors (IR-A, IR-B, IGF-1R, IGF-1R/IR-A, IGF-1R/IR-B), and IGF binding proteins (IGFBPs). The IGFBPs are a family of six proteins that bind IGFs in serum to transport and sequester IGFs, regulating availability to receptors. The effects of the two IGFs overlap as they are both potent mitogens; however, their relative potency depends on the cell type and the expression of the receptor types and isoforms. Quantitatively IGF-2 is the predominant circulating IGF present in adults, three times that of IGF-1. IGF-1R (Insulin-like Growth Factor-1 Receptor) is a transmembrane receptor that is activated by the IGFs and is involved in several important biological processes, including development and cell growth, proliferation and survival, as well as metabolism (regulation of glucose and lipid metabolism), and neurological function (regulation of synaptic plasticity, learning, and memory). IGF-2R is a transmembrane receptor that plays an important role in the regulation of growth and development, particularly during embryonic development. It is primarily involved in the bioavailability of IGF-2 (uptake and degradation/endocytosis-mediated clearance) thereby regulating IGF-2's activity and effects on cell growth and differentiation. IGF-2R is notable for having significantly higher affinity for IGF-2 than either IGF-1 or insulin. Insulin-like Growth Factor-2 (IGF-2) is a mitogenic peptide hormone that is structurally similar to insulin. IGF-2 is primarily produced by the liver but also by other tissues. IGF-2 plays an important role in the regulation of cell growth, differentiation, and metabolism. It binds to IGF-1R and the IR-A on the surface of target cells, leading to the activation of intracellular signaling pathways that promote cell proliferation, survival, and metabolism. Within the pathology of DM1, expression of the receptors for IGF-2 does not decline in aged or DM1 muscle precursor cells. Recent discoveries have demonstrated that the IGF-2R does more than just regulate IGF-2 concentration via endocytosis-mediated clearance. IGF-2R activation also drives autocrine S1P signaling and has also been shown to benefit muscle by activating phospholipase C to increase IP3 signaling and calcium flux, supporting muscle cell migration during fiber formation, increasing acetylcholine release, amino acid uptake, and switching macrophages to an anti-inflammatory state. HSA-IGF2-R61A has comparable affinity for IGF-1R and IGF-2R. In some embodiments of methods described herein, HSA-IGF2-R61A interacts with both receptors (IGF-1R and IGF-2R) in vivo.

### IGF Signaling and Adult-Onset DM1

In adult-onset DM1 there is a connection between deficits in IGF signaling and the pathogenesis and progression of the disease. IGF-1R is the primary receptor through which IGF-1 exerts its effects. IGF-2, similar to IGF-1, binds to and activates IGF-1R. This binding, in turn, activates several signaling pathways, including the PI3K/Akt and the Rat sarcoma virus (RAS)/MAPK pathways, which are involved in cell growth, survival, differentiation, and metabolism. In muscle pathology and aging, there is a predominant switch of the fiber phenotype from fast to slow. In a DM1 mouse model, IGF-2 was shown to orchestrate the development of fast myofibers by acting as a twitch motor unit during secondary myogenesis.

### HSA-IGF2 Fusion Proteins

In some aspects, an HSA-IGF2 fusion protein is administered to the subject to treat a muscle degeneration condition. In some embodiments, the muscle degeneration condition comprises a muscular dystrophy. In some embodiments, the muscle degeneration condition comprises a myotonic muscular dystrophy. In some embodiments, the muscle degeneration condition comprises DM1. In some embodiments, the muscle degeneration condition comprises DM2. In some embodiments, the muscle degeneration condition comprises sarcopenia. In some embodiments, the HSA-IGF2 fusion protein is formulated as a pharmaceutical composition. In some embodiments, the HSA-IGF2 fusion protein of the pharmaceutical composition comprises HSA-IGF2-R61A. In some embodiments, the HSA-IGF2-R61A comprises the amino acid sequence of SEQ ID NO: 1. In some embodiments, the HSA-IGF2 fusion protein of the pharmaceutical composition consists essentially of HSA-IGF2-R61A. In some embodiments, the HSA-IGF2-R61A consists essentially of the amino acid sequence of SEQ ID NO: 1. In some embodiments, the HSA-IGF2 fusion protein of the pharmaceutical composition comprises HSA-IGF2-R61Q. In some embodiments, the HSA-IGF2-R61Q comprises the amino acid sequence of SEQ ID NO: 2. In some embodiments, the HSA-IGF2 fusion protein of the pharmaceutical composition consists essentially of HSA-IGF2-R61Q. In some embodiments, the HSA-IGF2-R61Q consists essentially of the amino acid sequence of SEQ ID NO: 2. In some embodiments, the HSA-IGF2 fusion protein of the pharmaceutical composition comprises HSA-IGF2-R64A. In some embodiments, the HSA-IGF2-R64A comprises the amino acid sequence of SEQ ID NO: 3. In some embodiments, the HSA-IGF2 fusion protein of the pharmaceutical composition consists essentially of HSA-IGF2-R64A. In some embodiments, the HSA-IGF2-R64A consists essentially of the amino acid sequence of SEQ ID NO: 3. In some embodiments, the HSA-IGF2 fusion protein of the pharmaceutical composition comprises HSA-IGF2-R64Q. In some embodiments, the HSA-IGF2-R64Q comprises the amino acid sequence of SEQ ID NO: 4. In some embodiments, the HSA-IGF2 fusion protein of the pharmaceutical composition consists essentially of HSA-IGF2-R64Q. In some embodiments, the HSA-IGF2-R64Q consists essentially of the amino acid sequence of SEQ ID NO: 4.

### IGF-2 Signaling and Overview of HSA-IGF2-R61A and Mechanism of Action

IGF-2 is expressed in early fetal development and regulates cell proliferation, growth, migration, differentiation, and survival of several somatic tissue types, including skeletal muscle. IGF-2 activates the cell surface receptor tyrosine kinases, IGF-1R, IR, and IGF-2R, which is unique among insulin-like ligands. IGF-1R activation initiates the well-studied PI3K/AKT signaling pathway that inhibits atrophy and apoptosis, and in muscle contributes to myogenic differentiation by driving expression of the myogenic transcription factor (MyoD1), and which effects cell growth, survival, differentiation, and metabolism. IGF-2 has higher affinity for IR than IGF-1, particularly for the embryonic isoform of IR-A which has a lower affinity for glucose and is aberrantly expressed as the dominant IR isoform in DM1 and is driving diabetic phenotypes. In some aspects described herein, HSA-IGF2-R61A is a recombinant fusion protein (biologic) made by linking human serum albumen (HSA) to human IGF-2; HSA improves the molecule's half-life. In some embodiments, the HSA-IGF2-R61A comprises the amino acid sequence of SEQ ID NO: 1. In some embodiments, the HSA-IGF2-R61A consists essentially of the amino acid sequence of SEQ ID NO: 1. In some embodiments, HSA-IGF2-R61A functions as an agonist, following administration of a pharmaceutical composition comprising HSA-IGF2-R61A, that targets mitogen-activated protein kinase/extracellular signal-regulated kinase (MAPK/ERK) and phosphatidylinositol 3-kinase/serine/threonine protein kinase B (PI3K/AKT) regenerative pathways, the major signaling pathways in skeletal muscle, to enhance myogenesis, muscle survival and strength. HSA-IGF2-R61A is a recombinant fusion protein (biologic) comprised of HSA linked to a human insulin-like growth factor-2 sequence (HSA-IGF2-R61A). In some embodiments, HSA-IGF2-R61A comprises an amino acid linker region in between the HSA domain and the IGF2-R61A domain. In some embodiments, the amino acid linker region comprises Linker 1 having the amino acid sequence of SEQ ID NO: 18. In some embodiments, the amino acid linker region comprises Linker 2 having the amino acid sequence of SEQ ID NO: 19. In some embodiments, the amino acid linker region comprises Linker 3 having the amino acid sequence of SEQ ID NO: 20. In some embodiments, the amino acid linker region consists of Linker 3 having the amino acid sequence of SEQ ID NO: 20. In some embodiments, the HSA-IGF2-R61A administered to the subject is a single chain polypeptide comprising the amino acid sequence of SEQ ID NO: 1. In some embodiments, the HSA-IGF2-R61A administered to the subject is a single chain polypeptide consisting essentially of the amino acid sequence of SEQ ID NO: 1. In some embodiments, the HSA-IGF2-R61A administered to the subject is a single chain polypeptide consisting of the amino acid sequence of SEQ ID NO: 1. The drug class for HSA-IGF2-R61A is a fusion protein biologic.

### Methods of Use of Pharmaceutical Compositions

In some aspects disclosed herein, a pharmaceutical composition described here in used for the treatment of a muscle condition. In some embodiments, the muscle condition is sarcopenia. In some embodiments, the muscle condition is glucocorticoid-induced muscle atrophy. In some embodiments, the muscle condition is a muscular dystrophy. In some embodiments, the muscle condition is a muscle wasting muscular dystrophy. In some embodiments, the muscle condition is an aging disorder. In some embodiments, the muscle condition is a myotonic dystrophy. In some embodiments, the muscle condition is DM1. In some embodiments, the muscle condition is DM2. In some embodiments, the muscle condition is adult-onset DM1. In some embodiments, the muscle condition is congenital DM1. In some embodiments, the muscle condition is juvenile DM1. In some embodiments, the muscle condition is late-onset DM1.

In some aspects disclosed herein are methods of treating a subject having myotonic muscular dystrophy, comprising administering an effective amount of a fusion protein comprising the amino acid sequence of SEQ ID NO: 1 to the subject, wherein the subject has received a physician diagnosis of myotonic dystrophy type 1 (DM1) or myotonic dystrophy type 2 (DM2). In some aspects disclosed herein are methods of treating a subject having myotonic muscular dystrophy, comprising administering an effective amount of a fusion protein comprising the amino acid sequence of SEQ ID NO: 1 to the subject, wherein the subject has received a physician diagnosis of myotonic dystrophy type 1 (DM1). In some aspects disclosed herein are methods of treating a subject having myotonic muscular dystrophy, comprising administering an effective amount of a fusion protein comprising the amino acid sequence of SEQ ID NO: 1 to the subject, wherein the subject has received a physician diagnosis of myotonic dystrophy type 2 (DM2). In some embodiments, the administering comprises subcutaneous administration. In some embodiments, the administering comprises administration according to a dosage regime. In some embodiments, the dosage regime comprises once daily, once every other day, twice weekly, once weekly, once every ten days, once every two weeks, once every three weeks, once every month, once every two months, once every three months, once every four months, or once every six months administration of the fusion protein to the subject. In some embodiments, a pharmaceutical composition described herein is administered once daily to the subject. In some embodiments, a pharmaceutical composition described herein is administered once every other day to the subject. In some embodiments, a pharmaceutical composition described herein is administered twice weekly to the subject. In some embodiments, a pharmaceutical composition described herein is administered weekly to the subject. In some embodiments, a pharmaceutical composition described herein is administered weekly to the subject with one or more injections within a period of about 5-15 minutes. In some embodiments, a single injection of the pharmaceutical composition delivers a sufficient dosage to the subject. In some embodiments, multiple injections of the pharmaceutical composition deliver a sufficient dosage to the subject. In some embodiments, administration comprises a single sc injection of the pharmaceutical composition. In some embodiments, administration comprises two sc injections of the pharmaceutical composition. In some embodiments, administration comprises three sc injections of the pharmaceutical composition. In some embodiments, administration comprises four sc injections of the pharmaceutical composition. In some embodiments, administration comprises a single weekly sc injection of the pharmaceutical composition. In some embodiments, administration comprises two sc injections each week of the pharmaceutical composition wherein the two sc injections are administered within 15 minutes of each other. In some embodiments, administration comprises three sc injections each week of the pharmaceutical composition wherein the three sc injections are administered within 15 minutes of each other. In some embodiments, a pharmaceutical composition described herein is administered every other week to the subject.

In some embodiments, the effective amount of fusion protein comprising the amino acid sequence of SEQ ID NO: 1 is determined based on body weight and/or pharmacokinetic response of the subject. In some embodiments, dosage of the fusion protein administered to the subject is determined based on actual body of the subject at initiation of therapy. In some embodiments, the effective amount of the fusion protein is less than about 0.5, 0.75, 1, 1.25, 1.5, 1.75, 2.0, 2.25, 2.5, 2.75, 3, 3.5, 4, 4.5, or 5 mg/kg of body weight of the subject. In some embodiments, the effective amount of the fusion protein is greater than about 0.01, 0.02, 0.05, 0.1, 0.2, 0.3, 0.4, 0.5, 0.6, 0.7, 0.75, 0.8, 0.9, 1, 1.1, 1.25, 1.35, 1.5, 1.65, 1.75, 1.85, 2.0, 2.25, 2.5, 2.75, or 3 mg/kg of body weight of the subject. In some embodiments, the effective amount of the fusion protein is about 0.01, 0.02, 0.05, 0.1, 0.2, 0.3, 0.4, 0.5, 0.6, 0.7, 0.75, 0.8, 0.9, 1, 1.1, 1.25, 1.35, 1.4, 1.5, 1.6, 1.65, 1.7, 1.75, 1.85, 1.9, 2.0, 2.1, 2, 2, 2.25, 2.3, 2.4, 2.5, 2.6, 2.7, 2.75, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mg/kg of body weight of the subject. In some embodiments, the effective amount of the fusion protein is between about 0.5 to about 4 mg/kg of body weight of the subject. In some embodiments, the effective amount of the fusion protein is at least about 0.01 mg/kg of body weight of the subject. In some embodiments, the effective amount of the fusion protein is at least about 0.1 mg/kg of body weight of the subject. In some embodiments, the effective amount of the fusion protein is at least about 0.5 mg/kg of body weight of the subject. In some embodiments, the effective amount of the fusion protein is between about 0.1 to about 5 mg/kg of body weight of the subject. In some embodiments, the effective amount of the fusion protein is between about 0.25 to about 4 mg/kg of body weight of the subject. In some embodiments, the effective amount of the fusion protein is between about 1 to about 3 mg/kg of body weight of the subject. In some embodiments, the effective amount of the fusion protein administered to the subject is adjusted to achieve a peak serum concentration (Cₘₐₓ) following subcutaneous administration of less than or equal to about 50, 40, 30, 25, 20, 17.5, 15, 12.5, 10, 7.5, 5 or 3 μg/mL. In some embodiments, the effective amount of the fusion protein administered to the subject is adjusted to achieve a peak serum concentration (Cₘₐₓ) following subcutaneous administration of less than or equal to 15 μg/mL. In some embodiments, the effective amount of the fusion protein administered to the subject is adjusted to achieve a peak serum concentration (Cₘₐₓ) following subcutaneous administration of greater than or equal to about 1, 2, 3, 4, 5, 6, 7, 8, 9, 10, 11, 12, 13, 14, 15, 16, 17, 18, 19, 20, 22.5, 25, 27.5, or 30 μg/mL. In some embodiments, the effective amount of the fusion protein administered to the subject is adjusted to achieve a peak serum concentration (Cₘₐₓ) following subcutaneous administration of between about 5 to about 25 μg/mL. In some embodiments, an effective concentration is determined at least in part by converting information obtained in an animal model to identify an equivalent dosage in a human subject. In some embodiments, the conversion of effective dosage information is done according to a method described in Nair A B and Jacob S. A simple practice guide for dose conversion between animals and human. J Basic Clin Pharm. 2016 March; 7(2):27-31, wherein is hereby incorporated by reference in its entirety regarding methods describing dose conversion. In some embodiments, the pharmaceutical composition is administered to the subject at a dose of at least about 5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 210, 225, 240, 250, 260, 275, 290, 300, 325, 350, 375, 400, 425, 450 mg of the fusion protein. In some embodiments, the pharmaceutical composition is administered to the subject at a dose of at most about 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 210, 225, 240, 250, 260, 275, 290, 300, 325, 350, 375, 400, 425, 450, or 500 mg of the fusion protein. In some embodiments, the pharmaceutical composition is administered to the subject at a dose of at most about 5 mg to 500 mg of the fusion protein.

In some embodiments, the subject is assessed for DM1 symptoms and motor assessment prior to initiation of treatment. In some embodiments, the subject possesses an ability to walk independently for at least 10 meters at a time of screening prior to initiation of administration of the fusion protein. In some embodiments, the subject is assessed for the ability to walk independently with or without the use of orthoses or ankle braces.

In some embodiments, the subject has received a physician diagnosis of DM1 prior to initiation of treatment. In some embodiments, the subject receiving a physician diagnosis of DM1 has received genetic confirmation of DMPK repeat expansion. In some embodiments, DMPK repeat expansion comprises at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88., 89, 90, 91, 92, 93, 94, 95, S ‘6, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206,207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, :271, 272, :273, 274, :275, 276, :277, 278, :279, 280, 281,282, 283, 284, 285, 286, 287, 288, 289, 290, 291, 292,293, 294, 295, 296, 297, 298, 299, 300,301, 302, 303, 304, 305,306, 307, 308, 309,310, 311, 312, 313, 314,315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434 435, 436 437, 438, 439, 440, 441, 442, 443, 444, 445 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 510, 520, 530, 540, 550, 560, 570, 580, 590, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 CTG repeats in the DMPK gene. In some embodiments, DMPK repeat expansion comprises DMPK repeat expansion comprises greater than 100 CTG repeats in the DMPK gene.

In some aspects, administering an effective amount of a pharmaceutical composition described herein to a subject to treat myotonic dystrophy regulates signaling pathways in the subject following the administering. In some embodiments, fusion protein of the pharmaceutical composition agonistically promotes ERK1, ERK2, or ERK1 and ERK2 phosphorylation following the subcutaneous administration to the subject. In some embodiments, promotion of ERK1, ERK2, or ERK1 and ERK2 phosphorylation activates ERK1/2 downstream signaling following the subcutaneous administration to the subject. In some embodiments, the fusion protein of the pharmaceutical composition agonistically promotes mitogen-activated protein kinase kinase (MEK) pathway activation following the subcutaneous administration to the subject. In some embodiments, the fusion protein of the pharmaceutical composition agonistically activates PI3K signaling following the subcutaneous administration to the subject. In some embodiments, the fusion protein of the pharmaceutical composition agonistically activates AKT signaling following the subcutaneous administration to the subject. In some embodiments, the activation of PI3K signaling agonistically activates AKT signaling following the subcutaneous administration to the subject.

In some aspects, administering an effective amount of a pharmaceutical composition described herein to a subject to treat myotonic dystrophy produces a therapeutic benefit in the subject. In some embodiments, the administering promotes muscle cell differentiation in the subject. In some embodiments, the administering promotes muscle cell survival in the subject. In some embodiments, the promotion of muscle cell survival in the subject leads to a lower rate of muscle cell degeneration compared to a subject having myotonic muscular dystrophy that has not been administered the fusion protein. In some embodiments, the administering improves muscle strength in the subject. In some embodiments, the administering reduces muscle wasting in the subject.

In some embodiments, the method of treatment comprises subcutaneous injection of a pharmaceutical composition described herein in the subject. In some embodiments, the pharmaceutical composition is administered with a syringe that contains 0.02 mL graduations.

In some embodiments, the method of treatment comprises administering to a subjecting having DM1, wherein the subject is at least a certain age. In some embodiments, the age of onset of DM1 symptoms in the subject is used to determine DM1 phenotype. In some embodiments, determination of DM1 phenotype in the subject is used to assign an HSA-IGF2-R61A treatment regimen and/or dosage schedule to the subject. In some embodiments, the subject is at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years of age. In some embodiments, the subject is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years of age. In some embodiments, the subject is about 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years of age. In some embodiments, the subject is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 years of age. In some embodiments, the age range of the subject is 18-60. In some embodiments, the age range of the subject is 20-60. In some embodiments, the subject is at least 11 years of age. In some embodiments, the subject is at least 18 years of age. In some embodiments, the subject is determined to have late-onset DM1 and is at least about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years of age. In some embodiments, the subject is determined to have late-onset DM1 and is 40 years of age or older. In some embodiments, the subject is determined to have adult-onset “classical DM1” and is between about 20-40 years of age. In some embodiments, the subject is determined to have adult-onset “classical DM1” and is 20, 21, 22, 23, 24, 25, 26, 27, 28, 29, 30, 31, 32, 33, 34, 35, 36, 37, 38, 39, or 40 years of age. In some embodiments, the subject is determined to have adult-onset “classical DM1” and is 20 years of age or older. In some embodiments, the subject is determined to have adult-onset “classical DM1” and is 18 years of age or older. In some embodiments, the subject is determined to have juvenile DM1 and is between about 10-20 years of age. In some embodiments, the subject is determined to have juvenile DM1 and is 10, 11, 12, 13, 14, 15, 16, 17, 18, 19, or 20 years of age. In some embodiments, the subject is determined to have infantile DM1 and is between about 1 month to 10 years of age. In some embodiments, the subject is determined to have infantile DM1 and is 1 month, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years or 10 years of age. In some embodiments, the subject is determined to have congenital DM1 evident from birth.

In some aspects of methods described herein, toxicology studies have been undertaken to assess any potential harmful effects of administering pharmaceutical compositions comprising HSA-IGF2-R61A described herein.

### Use in Specific Populations

In some instances, the clinical development program for HSA-IGF2-R61A enrolls symptomatic adult-onset phenotype subjects 18 years of age or older with documented genetic confirmation of DM1 DMPK gene CTG repeat length ≥100. Based on registry data, adult-onset and late-onset phenotypes together account for approximately 50% of the DM1 population. Thus, the specific target population for development of HSA-IGF2-R61A is approximately 80,000 patients in the United States (overall population 336,000,000) and approximately 9-10,000 patients in Canada (overall population 39,000,000). In some instances, the number of CTG repeats within the DMPK gene is correlated with the various DM1 phenotypes, disease severity, and age of onset and is used in the diagnosis of DM1. In some instances, the number of CTG repeats within the DMPK gene is correlated with the various DM1 phenotypes, disease severity, and age of onset and is used as a patient selection criterion for administration of HSA-IGF2-R61A. In some instances, age of onset, most prominent clinical symptoms, and/or categorization of disease phenotype or disease severity are further used as patient selection criteria for administration of HSA-IGF2-R61A. In some instances, CTG repeats within the DMPK gene according to the range listed in Table 2 are used as a patient selection criterion for administration of HSA-IGF2-R61A. In some instances, a patient is selected for administration of HSA-IGF2-R61A by determining a number of CTG repeats within the DMPK gene. In some instances, the patient selected for administration of HSA-IGF2-R61A is determined to have at least about 100, 110, 120, 130, 140, 150, 160, 170, 180, 190, 200, 220, 240, 250, 260, 280, 300, 320, 340, 360, 380, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1150, 1200, 1250, 1300, 1350, or 1400 or more. In some instances, the patient selected for administration of HSA-IGF2-R61A is determined to have between about 100-600 CTG repeats within the DMPK gene and is classified with a late-onset DM1 phenotype. In some instances, the patient selected for administration of HSA-IGF2-R61A is determined to have between about 250-750 CTG repeats within the DMPK gene and is classified with an adult-onset “classical DM1” phenotype. In some instances, the patient selected for administration of HSA-IGF2-R61A is determined to have between about 400-800 CTG repeats within the DMPK gene and is classified with a juvenile DM1 phenotype. In some instances, the patient selected for administration of HSA-IGF2-R61A is determined to have between about 500-1100 CTG repeats within the DMPK gene and is classified with a Infantile DM1 phenotype. In some instances, the patient selected for administration of HSA-IGF2-R61A is determined to have between about 750-1400 CTG repeats within the DMPK gene and is classified with a Congenital DM1 phenotype. In some instances, age of onset of symptoms listed in Table 2 is used as a patient selection criterion for administration of HSA-IGF2-R61A. In some instances, the patient selected for administration of HSA-IGF2-R61A is determined to have a late-onset DM1 phenotype with an age of onset of greater than 40 years of age. In some instances, the patient selected for administration of HSA-IGF2-R61A is determined to have an adult-onset “classical DM1” phenotype with an age of onset of between 20-40 years of age. In some instances, the patient selected for administration of HSA-IGF2-R61A is determined to have a juvenile DM1 phenotype with an age of onset of between 10-20 years of age. In some instances, the patient selected for administration of HSA-IGF2-R61A is determined to have an infantile DM1 phenotype with an age of onset of between 1 month and 10 years of age. In some instances, the patient selected for administration of HSA-IGF2-R61A is determined to have a congenital DM1 phenotype with an age of onset at birth. In some instances, presence of one or more of the most prominent clinical symptoms listed in Table 2 is used as a patient selection criterion for administration of HSA-IGF2-R61A. In some instances, diagnosis of a DM1 phenotype as listed in Table 2 is used as a patient selection criterion for administration of HSA-IGF2-R61A. In some instances, the patient selected for administration of HSA-IGF2-R61A is determined to have one or more of the affected organ symptoms listed in Table 3A and/or Table 3B characterized by one or more of the listed deficiencies for that organ system. In some instances, the patient selected for administration of HSA-IGF2-R61A is determined to have one or more DM1-related deficiencies within muscle, the CNS, the visual system, the endocrine system, the respiratory system, the skin, the immune system, the male reproductive system, the female reproductive system, the skeletal system, the cardiovascular system, and/or the gastrointestinal system. In some instances, the patient selected for administration of HSA-IGF2-R61A is determined to have one or more DM1-related deficiencies within muscle. In some instances, the effect of life span listed in Table 2 is used as a patient selection criterion for administration of HSA-IGF2-R61A and/or assignment of an HSA-IGF2-R61A administration schedule and/or dosage regimen to the subject. In some instance, patient selection for administration of HSA-IGF2-R61A is based on one or more criteria listed in Table 2. In some instance, patient selection for administration of HSA-IGF2-R61A is based on one or more criteria listed in Table 3A and/or Table 3B.

Amino acid sequences for fusion proteins, and elements used in construction of the fusion proteins, for use in the pharmaceutical compositions described here are listed below in Table 4.

TABLE 4
Protein and peptide sequences
Table of Sequences

| SEQ ID | Sequence | | |
|---|---|---|---|
| NO: | name | Description | Sequence |
| 1 | HSA- | HSA fusion | SGDAHKSEVAHRFKDLGEENFKALVLIAFAQYLQQCPFEDHVKLVNE |
| | IGF2- | IGF2 R61A | VTEFAKTCVADESAENCDKSLHTLFGDKLCTVATLRETYGEMADCC |
| | R61A | | AKQEPERNECFLQHKDDNPNLPRLVRPEVDVMCTAFHDNEETFLKK |
| | | | YLYEIARRHPYFYAPELLFFAKRYKAAFTECCQAADKAACLLPKLDE |
| | | | LRDEGKASSAKQRLKCASLQKFGERAFKAWAVARLSQRFPKAEFAE |
| | | | VSKLVTDLTKVHTECCHGDLLECADDRADLAKYICENQDSISSKLKE |
| | | | CCEKPLLEKSHCIAEVENDEMPADLPSLAADFVESKDVCKNYAEAKD |
| | | | VFLGMFLYEYARRHPDYSVVLLLRLAKTYETTLEKCCAAADPHECY |
| | | | AKVFDEFKPLVEEPQNLIKQNCELFEQLGEYKFQNALLVRYTKKVPQ |
| | | | VSTPTLVEVSRNLGKVGSKCCKHPEAKRMPCAEDYLSVVLNQLCVL |
| | | | HEKTPVSDRVTKCCTESLVNRRPCFSALEVDETYVPKEFNAETFTFHA |
| | | | DICTLSEKERQIKKQTALVELVKHKPKATKEQLKAVMDDFAAFVEKC |
| | | | CKADDKETCFAEEGKKLVAASQAALGLGGGGSGGGGSGGGGSAYR |
| | | | PSETLCGGELVDTLQFVCGDRGFYFSRPASRVSARSRGIVEECCFRSC |
| | | | DLALLETYCATPAKSE |
| 2 | HSA- | HSA fusion | SGDAHKSEVAHRFKDLGEENFKALVLIAFAQYLQQCPFEDHVKLVNE |
| | IGF2- | IGF2 R61Q | VTEFAKTCVADESAENCDKSLHTLFGDKLCTVATLRETYGEMADCC |
| | R61Q | | AKQEPERNECFLQHKDDNPNLPRLVRPEVDVMCTAFHDNEETFLKK |
| | | | YLYEIARRHPYFYAPELLFFAKRYKAAFTECCQAADKAACLLPKLDE |
| | | | LRDEGKASSAKQRLKCASLQKFGERAFKAWAVARLSQRFPKAEFAE |
| | | | VSKLVTDLTKVHTECCHGDLLECADDRADLAKYICENQDSISSKLKE |
| | | | CCEKPLLEKSHCIAEVENDEMPADLPSLAADFVESKDVCKNYAEAKD |
| | | | VFLGMFLYEYARRHPDYSVVLLLRLAKTYETTLEKCCAAADPHECY |
| | | | AKVFDEFKPLVEEPQNLIKQNCELFEQLGEYKFQNALLVRYTKKVPQ |
| | | | VSTPTLVEVSRNLGKVGSKCCKHPEAKRMPCAEDYLSVVLNQLCVL |
| | | | HEKTPVSDRVTKCCTESLVNRRPCFSALEVDETYVPKEFNAETFTFHA |
| | | | DICTLSEKERQIKKQTALVELVKHKPKATKEQLKAVMDDFAAFVEKC |
| | | | CKADDKETCFAEEGKKLVAASQAALGLGGGGSGGGGSGGGGSAYR |
| | | | PSETLCGGELVDTLQFVCGDRGFYFSRPASRVSQRSRGIVEECCFRSC |
| | | | DLALLETYCATPAKSE |
| 3 | HSA- | HSA fusion | SGDAHKSEVAHRFKDLGEENFKALVLIAFAQYLQQCPFEDHVKLVNE |
| | IGF2- | IGF2 R64A | VTEFAKTCVADESAENCDKSLHTLFGDKLCTVATLRETYGEMADCC |
| | R64A | | AKQEPERNECFLQHKDDNPNLPRLVRPEVDVMCTAFHDNEETFLKK |
| | | | YLYEIARRHPYFYAPELLFFAKRYKAAFTECCQAADKAACLLPKLDE |
| | | | LRDEGKASSAKQRLKCASLQKFGERAFKAWAVARLSQRFPKAEFAE |
| | | | VSKLVTDLTKVHTECCHGDLLECADDRADLAKYICENQDSISSKLKE |
| | | | CCEKPLLEKSHCIAEVENDEMPADLPSLAADFVESKDVCKNYAEAKD |
| | | | VFLGMFLYEYARRHPDYSVVLLLRLAKTYETTLEKCCAAADPHECY |
| | | | AKVFDEFKPLVEEPQNLIKQNCELFEQLGEYKFQNALLVRYTKKVPQ |
| | | | VSTPTLVEVSRNLGKVGSKCCKHPEAKRMPCAEDYLSVVLNQLCVL |
| | | | HEKTPVSDRVTKCCTESLVNRRPCFSALEVDETYVPKEFNAETFTFHA |
| | | | DICTLSEKERQIKKQTALVELVKHKPKATKEQLKAVMDDFAAFVEKC |
| | | | CKADDKETCFAEEGKKLVAASQAALGLGGGGSGGGGSGGGGSAYR |
| | | | PSETLCGGELVDTLQFVCGDRGFYFSRPASRVSRRSAGIVEECCFRSC |
| | | | DLALLETYCATPAKSE |
| 4 | HSA- | HSA fusion | SGDAHKSEVAHRFKDLGEENFKALVLIAFAQYLQQCPFEDHVKLVNE |
| | IGF2- | IGF2 R64Q | VTEFAKTCVADESAENCDKSLHTLFGDKLCTVATLRETYGEMADCC |
| | R64Q | | AKQEPERNECFLQHKDDNPNLPRLVRPEVDVMCTAFHDNEETFLKK |
| | | | YLYEIARRHPYFYAPELLFFAKRYKAAFTECCQAADKAACLLPKLDE |
| | | | LRDEGKASSAKQRLKCASLQKFGERAFKAWAVARLSQRFPKAEFAE |
| | | | VSKLVTDLTKVHTECCHGDLLECADDRADLAKYICENQDSISSKLKE |
| | | | CCEKPLLEKSHCIAEVENDEMPADLPSLAADFVESKDVCKNYAEAKD |
| | | | VFLGMFLYEYARRHPDYSVVLLLRLAKTYETTLEKCCAAADPHECY |
| | | | AKVFDEFKPLVEEPQNLIKQNCELFEQLGEYKFQNALLVRYTKKVPQ |
| | | | VSTPTLVEVSRNLGKVGSKCCKHPEAKRMPCAEDYLSVVLNQLCVL |
| | | | HEKTPVSDRVTKCCTESLVNRRPCFSALEVDETYVPKEFNAETFTFHA |
| | | | DICTLSEKERQIKKQTALVELVKHKPKATKEQLKAVMDDFAAFVEKC |
| | | | CKADDKETCFAEEGKKLVAASQAALGLGGGGSGGGGSGGGGSAYR |
| | | | PSETLCGGELVDTLQFVCGDRGFYFSRPASRVSRRSQGIVEECCFRSC |
| | | | DLALLETYCATPAKSE |
| 5 | Pro- | Pro-HSA fusion | RGVFRRSGDAHKSEVAHRFKDLGEENFKALVLIAFAQYLQQCPFEDH |
| | HSA- | IGF2 R61A | VKLVNEVTEFAKTCVADESAENCDKSLHTLFGDKLCTVATLRETYGE |
| | IGF2- | | MADCCAKQEPERNECFLQHKDDNPNLPRLVRPEVDVMCTAFHDNEE |
| | R61A | | TFLKKYLYEIARRHPYFYAPELLFFAKRYKAAFTECCQAADKAACLL |
| | | | PKLDELRDEGKASSAKQRLKCASLQKFGERAFKAWAVARLSQRFPK |
| | | | AEFAEVSKLVTDLTKVHTECCHGDLLECADDRADLAKYICENQDSIS |
| | | | SKLKECCEKPLLEKSHCIAEVENDEMPADLPSLAADFVESKDVCKNY |
| | | | AEAKDVFLGMFLYEYARRHPDYSVVLLLRLAKTYETTLEKCCAAAD |
| | | | PHECYAKVFDEFKPLVEEPQNLIKQNCELFEQLGEYKFQNALLVRYT |
| | | | KKVPQVSTPTLVEVSRNLGKVGSKCCKHPEAKRMPCAEDYLSVVLN |
| | | | QLCVLHEKTPVSDRVTKCCTESLVNRRPCFSALEVDETYVPKEFNAE |
| | | | TFTFHADICTLSEKERQIKKQTALVELVKHKPKATKEQLKAVMDDFA |
| | | | AFVEKCCKADDKETCFAEEGKKLVAASQAALGLGGGGSGGGGSGG |
| | | | GGSAYRPSETLCGGELVDTLQFVCGDRGFYFSRPASRVSARSRGIVEE |
| | | | CCFRSCDLALLETYCATPAKSE |
| 6 | Pro- | Pro-HSA fusion | RGVFRRSGDAHKSEVAHRFKDLGEENFKALVLIAFAQYLQQCPFEDH |
| | HSA- | IGF2 R61Q | VKLVNEVTEFAKTCVADESAENCDKSLHTLFGDKLCTVATLRETYGE |
| | IGF2- | | MADCCAKQEPERNECFLQHKDDNPNLPRLVRPEVDVMCTAFHDNEE |
| | R61Q | | TFLKKYLYEIARRHPYFYAPELLFFAKRYKAAFTECCQAADKAACLL |
| | | | PKLDELRDEGKASSAKQRLKCASLQKFGERAFKAWAVARLSQRFPK |
| | | | AEFAEVSKLVTDLTKVHTECCHGDLLECADDRADLAKYICENQDSIS |
| | | | SKLKECCEKPLLEKSHCIAEVENDEMPADLPSLAADFVESKDVCKNY |
| | | | AEAKDVFLGMFLYEYARRHPDYSVVLLLRLAKTYETTLEKCCAAAD |
| | | | PHECYAKVFDEFKPLVEEPQNLIKQNCELFEQLGEYKFQNALLVRYT |
| | | | KKVPQVSTPTLVEVSRNLGKVGSKCCKHPEAKRMPCAEDYLSVVLN |
| | | | QLCVLHEKTPVSDRVTKCCTESLVNRRPCFSALEVDETYVPKEFNAE |
| | | | TFTFHADICTLSEKERQIKKQTALVELVKHKPKATKEQLKAVMDDFA |
| | | | AFVEKCCKADDKETCFAEEGKKLVAASQAALGLGGGGSGGGGSGG |
| | | | GGSAYRPSETLCGGELVDTLQFVCGDRGFYFSRPASRVSQRSRGIVEE |
| | | | CCFRSCDLALLETYCATPAKSE |
| 7 | Pro- | Pro-HSA fusion | RGVFRRSGDAHKSEVAHRFKDLGEENFKALVLIAFAQYLQQCPFEDH |
| | HSA- | IGF2 R64A | VKLVNEVTEFAKTCVADESAENCDKSLHTLFGDKLCTVATLRETYGE |
| | IGF2- | | MADCCAKQEPERNECFLQHKDDNPNLPRLVRPEVDVMCTAFHDNEE |
| | R64A | | TFLKKYLYEIARRHPYFYAPELLFFAKRYKAAFTECCQAADKAACLL |
| | | | PKLDELRDEGKASSAKQRLKCASLQKFGERAFKAWAVARLSQRFPK |
| | | | AEFAEVSKLVTDLTKVHTECCHGDLLECADDRADLAKYICENQDSIS |
| | | | SKLKECCEKPLLEKSHCIAEVENDEMPADLPSLAADFVESKDVCKNY |
| | | | AEAKDVFLGMFLYEYARRHPDYSVVLLLRLAKTYETTLEKCCAAAD |
| | | | PHECYAKVFDEFKPLVEEPQNLIKQNCELFEQLGEYKFQNALLVRYT |
| | | | KKVPQVSTPTLVEVSRNLGKVGSKCCKHPEAKRMPCAEDYLSVVLN |
| | | | QLCVLHEKTPVSDRVTKCCTESLVNRRPCFSALEVDETYVPKEFNAE |
| | | | TFTFHADICTLSEKERQIKKQTALVELVKHKPKATKEQLKAVMDDFA |
| | | | AFVEKCCKADDKETCFAEEGKKLVAASQAALGLGGGGSGGGGSGG |
| | | | GGSAYRPSETLCGGELVDTLQFVCGDRGFYFSRPASRVSRRSAGIVEE |
| | | | CCFRSCDLALLETYCATPAKSE |
| 8 | Pro- | Pro-HSA fusion | RGVFRRSGDAHKSEVAHRFKDLGEENFKALVLIAFAQYLQQCPFEDH |
| | HSA- | IGF2 R64Q | VKLVNEVTEFAKTCVADESAENCDKSLHTLFGDKLCTVATLRETYGE |
| | IGF2- | | MADCCAKQEPERNECFLQHKDDNPNLPRLVRPEVDVMCTAFHDNEE |
| | R64Q | | TFLKKYLYEIARRHPYFYAPELLFFAKRYKAAFTECCQAADKAACLL |
| | | | PKLDELRDEGKASSAKQRLKCASLQKFGERAFKAWAVARLSQRFPK |
| | | | AEFAEVSKLVTDLTKVHTECCHGDLLECADDRADLAKYICENQDSIS |
| | | | SKLKECCEKPLLEKSHCIAEVENDEMPADLPSLAADFVESKDVCKNY |
| | | | AEAKDVFLGMFLYEYARRHPDYSVVLLLRLAKTYETTLEKCCAAAD |
| | | | PHECYAKVFDEFKPLVEEPQNLIKQNCELFEQLGEYKFQNALLVRYT |
| | | | KKVPQVSTPTLVEVSRNLGKVGSKCCKHPEAKRMPCAEDYLSVVLN |
| | | | QLCVLHEKTPVSDRVTKCCTESLVNRRPCFSALEVDETYVPKEFNAE |
| | | | TFTFHADICTLSEKERQIKKQTALVELVKHKPKATKEQLKAVMDDFA |
| | | | AFVEKCCKADDKETCFAEEGKKLVAASQAALGLGGGGSGGGGSGG |
| | | | GGSAYRPSETLCGGELVDTLQFVCGDRGFYFSRPASRVSRRSQGIVEE |
| | | | CCFRSCDLALLETYCATPAKSE |
| 9 | IGF2 | IGF2 mature | AYRPSETLCGGELVDTLQFVCGDRGFYFSRPASRVSARSRGIVEECCF |
| | mature | peptide R61A | RSCDLALLETYCATPAKSE |
| | peptide | | |
| | R61A | | |
| 10 | IGF2 | IGF2 mature | AYRPSETLCGGELVDTLQFVCGDRGFYFSRPASRVSQRSRGIVEECCF |
| | mature | peptide R61Q | RSCDLALLETYCATPAKSE |
| | peptide | | |
| | R61Q | | |
| 11 | IGF2 | IGF2 mature | AYRPSETLCGGELVDTLQFVCGDRGFYFSRPASRVSRRSAGIVEECCF |
| | mature | peptide R64A | RSCDLALLETYCATPAKSE |
| | peptide | | |
| | R64A | | |
| 12 | IGF2 | IGF2 mature | AYRPSETLCGGELVDTLQFVCGDRGFYFSRPASRVSRRSQGIVEECCF |
| | mature | peptide R64Q | RSCDLALLETYCATPAKSE |
| | peptide | | |
| | R64Q | | |
| 13 | IGF2 | IGF2 mature | AYRPSETLCGGELVDTLQFVCGDRGFYFSRPASRVSRRSRGIVEECCF |
| | mature | hormone | RSCDLALLETYCATPAKSE |
| | peptide | peptide | |
| 14 | IGF2 | IGF2 | RDVSTPPTVLPDNFPRYPVGKFFQYDTWKQSTQRLRRGLPALLRARR |
| | pro- | propeptide | GHVLAKELEAFREAKRHRPLIALPTQDPAHGGAPPEMASNRK |
| | peptide | amino acid | |
| | | sequence | |
| 15 | IGF2 | Full-length | MGIPMGKSMLVLLTFLAFASCCIAAYRPSETLCGGELVDTLQFVCGD |
| | prepro | Human IGF2 | RGFYFSRPASRVSRRSRGIVEECCFRSCDLALLETYCATPAKSERDVS |
| | protein | | TPPTVLPDNFPRYPVGKFFQYDTWKQSTQRLRRGLPALLRARRGHVL |
| | | | AKELEAFREAKRHRPLIALPTQDPAHGGAPPEMASNRK |
| 16 | IGF2 | IGF2 C- | RGFYFSRPASRVSRRSR |
| | C- | Domain region | |
| | Domain | for mutations | |
| | | to alter | |
| | | cleavage | |
| 17 | HSA | Full length | DAHKSEVAHRFKDLGEENFKALVLIAFAQYLQQCPFEDHVKLVNEV |
| | | Human Serum | TEFAKTCVADESAENCDKSLHTLFGDKLCTVATLRETYGEMADCCA |
| | | Albumin amino | KQEPERNECFLQHKDDNPNLPRLVRPEVDVMCTAFHDNEETFLKKY |
| | | acid sequence | LYEIARRHPYFYAPELLFFAKRYKAAFTECCQAADKAACLLPKLDEL |
| | | | RDEGKASSAKQRLKCASLQKFGERAFKAWAVARLSQRFPKAEFAEV |
| | | | SKLVTDLTKVHTECCHGDLLECADDRADLAKYICENQDSISSKLKEC |
| | | | CEKPLLEKSHCIAEVENDEMPADLPSLAADFVESKDVCKNYAEAKDV |
| | | | FLGMFLYEYARRHPDYSVVLLLRLAKTYETTLEKCCAAADPHECYA |
| | | | KVFDEFKPLVEEPQNLIKQNCELFEQLGEYKFQNALLVRYTKKVPQV |
| | | | STPTLVEVSRNLGKVGSKCCKHPEAKRMPCAEDYLSVVLNQLCVLH |
| | | | EKTPVSDRVTKCCTESLVNRRPCFSALEVDETYVPKEFNAETFTFHAD |
| | | | ICTLSEKERQIKKQTALVELVKHKPKATKEQLKAVMDDFAAFVEKCC |
| | | | KADDKETCFAEEGKKLVAASQAALGL |
| 18 | Linker | A short flexible | GSGS |
| | 1 | linker amino | |
| | | acid sequence | |
| 19 | Linker | A short flexible | GSGSA |
| | 2 | linker amino | |
| | | acid sequence | |
| 20 | Linker | A long flexible | GGGGSGGGGSGGGGS |
| | 3 | linker amino | |
| | | acid sequence | |

### Articles of Manufacture

In embodiment of the invention described herein, an article of manufacture, or “kit”, containing materials useful for the treatment of the DM1 is provided. In one embodiment, the kit comprises a container comprising a pharmaceutical composition comprising HSA-IGF2-R61A. The kit may further comprise a label or package insert on or associated with the container The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, or pre-filled syringes. The container may be formed from a variety of materials such as glass or plastic. The container may hold a pharmaceutical composition comprising a formulation of HSA-IGF2-R61A which is effective for treating DM1 and may have a sterile access port (for example, the container may be an intravenous solution bag or a vial having a stopper pierceable by a hypodermic injection needle). The label or package insert indicates that the pharmaceutical composition is used for treating the condition of choice, such as muscular dystrophy. The label or package insert indicates that the pharmaceutical composition is used for treating the condition of choice, such as myotonic muscular dystrophy. The label or package insert indicates that the pharmaceutical composition is used for treating the condition of choice, such as DM1. In addition, the label or package insert may indicate that the patient to be treated is one having a muscular dystrophy such as DM1. In addition, the label or package insert may indicate that the patient to be treated is one having adult-onset DM1. The label or package insert may also indicate that the composition can be used to treat other disorders. Alternatively, or additionally, the article of manufacture may further comprise a second container comprising a pharmaceutically acceptable buffer, such as bacteriostatic water for injection, phosphate-buffered saline, Ringer's solution and dextrose solution. The kit may further include other materials desirable from a commercial and user standpoint, including other buffers, diluents, filters, needles, and syringes. In some embodiments, the syringe is a pre-filled syringe. The kit may further comprise directions for the administration of the pharmaceutical composition. In some embodiments, the pre-filled syringe has a set volume of about 0.4 mL, about 0.8 mL, about 1.0 mL, about 1.2 mL, or about 1.6 mL. In some embodiments, the pre-filled syringe is configured to deliver a dosage of about 0.4, 0.5, 1.0, 1.5, or 2.0 mg/kg for an 80 kg subject. In some embodiments, the pre-filled syringe contains a formulation concentration of about 40 mg/mL of pharmaceutical composition comprising HSA-IGF2-R61A. In some embodiments, the pre-filled syringe contains a formulation concentration of about 100 mg/mL of pharmaceutical composition comprising HSA-IGF2-R61A.

In one embodiment, a kit may comprise (a) a first container with a pharmaceutical composition comprising HSA-IGF2-R61A contained therein; and optionally (b) a second container with a second pharmaceutical formulation contained therein, wherein the second pharmaceutical formulation comprises a second compound with activity to preserve muscle strength and function. Alternatively, or additionally, the kit may further comprise a third container comprising a pharmaceutically-acceptable buffer, such as bacteriostatic water for injection, phosphate-buffered saline, Ringer's solution and dextrose solution. It may further include other materials desirable from a commercial and user standpoint, including other buffers, diluents, filters, needles, and syringes.

In an aspect described herein, are kits comprising a pharmaceutical composition described here and a label In some embodiments, the pharmaceutical composition comprises a stabilizer. In some embodiments, the pharmaceutical composition comprises a surfactant. In some embodiments, the pharmaceutical composition comprises a salt. In some embodiments, the pharmaceutical composition comprises a buffer. In some embodiments, the pharmaceutical composition comprises a pharmaceutically acceptable excipient, carrier, or diluent. In some embodiments, the kit comprises: (i) a pharmaceutical composition comprising a fusion protein comprising the amino acid sequence of SEQ ID NO: 1, and a label. In some embodiments, the label provides directions for use. In some embodiments, the label comprises a description of the product. In some embodiments, the label comprises instructions for use. In some embodiments, instruction for use provide information relating to any of: HSA-IGF2-R61A concentration, HSA-IGF2-R61A dosing information, methods of administering a pharmaceutical composition comprising HSA-IGF2-R61A, administration frequency, administration interval, treatment indications, symptoms to be treated by use of the product, symptoms associated with adult-onset myotonic dystrophy, method of delivery of the product by subcutaneous injection, contraindications, listing of metabolic abnormalities, time of maximum concentration (Tmax) following SC administration, listing of warnings and precautions, potential toxicities, potential immunogenicity, potential adverse drug reactions (ADRs), drug-drug interactions (DDIs), descriptions of use in specific populations, instructions for use in adults, a summary a clinical studies relating to the product, information relating to overdosage, storage conditions of the product, details of formulation of pharmaceutical composition, a summary of primary pharmacology, safety pharmacology, pharmacokinetics, and/or toxicology, a summary of nonclinical study information, or an indication that no serious adverse reactions (SARs) have been documented. In some embodiments, the description of the product is a recombinant fusion protein (biologic) made by linking HSA to a human IGF-2 sequence (HSA-IGF2-R61A). In some embodiments, the label comprises a listing of indications and use. In some embodiments, the listing of indications and use are that HSA-IGF2-R61A is for the treatment of symptoms associated with adult-onset myotonic dystrophy. In some embodiments, the label comprises a dosage and administration information. In some embodiments, the dosage and administration information indicate that HSA-IGF2-R61A is supplied in a vial or vials and that administration is by subcutaneous injection. In some embodiments, the label indicates that administration is to be done in a fed state. In some embodiments, the label indicates that dosing frequency is once weekly. In some embodiments, the label indicates that it is preferred that administration be done in the abdomen. In some embodiments, the label indicates that in case multiple administrations are required for specified dose strengths, all drug are to be administered within a 10-minute period. In some embodiments, the label comprises a listing of contraindications. In some embodiments, the contraindications state that HSA-IGF2-R61A administration is contraindicated in individuals allergic to any of the excipients used in the HSA-IGF2-R61A formulation. In some embodiments, the label comprises a listing of metabolic abnormalities. In some embodiments, the listing of metabolic abnormalities state that HSA-IGF2-R61A functions as an agonist, targeting MAPK/ERK and PI3K/AKT regenerative pathways, to enhance myogenesis, muscle survival and strength. In some embodiments, the listing of metabolic abnormalities state that administration of HSA-IGF2-R61A may be associated with changes in systemic glucose and/or insulin levels. In some embodiments, the listing of metabolic abnormalities state that subjects receiving HSA-IGF2-R61A are to be monitored for symptoms and signs of hypoglycemia. It is anticipated that the time of maximum concentration (Tmax) of HSA-IGF2-R61A following SC administration will be approximately 29 hours following the dose. In some embodiments, the listing of metabolic abnormalities state that subjects are to be observed for shakiness or jitteriness, fatigue, dizziness, confusion, irritability and difficulty concentrating. Lower glucose levels may be associated with sweating. If symptoms occur, approximately 15 g of glucose are to be provided to the subject with continued observation until symptoms abate. Glucose may be provided as glucose tablets, regular soda, hard candy, fruit juice, syrup or honey. In some embodiments, the label comprises a listing of warnings and precautions. In some embodiments, the warnings and precautions state that the first administration of HSA-IGF2-R61A to human subjects, it is important that subjects be observed in a monitored setting for the first six hours following administration of HSA-IGF2-R61A. In some embodiments, the warnings and precautions indicate potential toxicities. In some embodiments, the warnings and precautions indicate immunogenicity. In some embodiments, the warnings and precautions indicating immunogenicity state that as human protein therapeutics can induce immune responses in animal species, the development of the relevant ADA can limit the evaluation of the study outcomes with effect on PK (anti-isotypic and anti-idiotypic antibodies) and PD (anti-idiotypic antibodies) in various animal species. In some embodiments, the label comprises a listing of potential adverse drug reactions (ADRs). In some embodiments, the listing of potential ADRs comprises one or more of the following: hypoglycemia., injection site reactions, hemorrhage, or kidney toxicity in some embodiments, the hemorrhage listed as a potential ADR is gastrointestinal hemorrhage. In some embodiments, the gastrointestinal hemorrhage is secondary to immunogenicity. In some embodiments, the kidney toxicity is listed as associated with elevated BUN and creatinine levels and the presence of protein and bilirubin in the urine. In some embodiments., the label comprises a listing of drug interactions. In some embodiments, the listing of drug interactions indicates that the is no indication that HSA-IGF2-R61A upregulates or downregulates cytochrome P450 enzymes (CYPs). In some embodiments, the label indicates that notable drug-drug interactions (DDIs) are unlikely with HSA-IGF2-R61A. In some embodiments, the listing of drug interactions indicates that HSA-IGF2-R61A interacts with blood glucose-regulating medication (e.g., insulin or metformin). In some embodiments, the listing of drug interactions indicates that HSA-IGF2-R61A may affect blood glucose levels. In some embodiments, the label comprises a listing of uses in specific populations. In some embodiments, the uses in specific populations indicate uses during pregnancy and lactation. In some embodiments, the label indicates that female subjects administered HSA-IGF2-R61A are to avoid becoming pregnant. In some embodiments, the label indicates that male subjects administered HSA-IGF2-R61A are to avoid impregnating a female partner. In some embodiments, the label indicates that female and male subjects are to use effective methods of contraception through defined periods during and after treatment with HSA-IGF2-R61A. In some embodiments, the label indicates that if a female subject becomes pregnant during HSA-IGF2-R61A treatment, HSA-IGF2-R61A therapy are to be stopped, and the patient is to be informed of the potential hazards to the fetus. In some embodiments, the label indicates that if a male subject's partner becomes pregnant during HSA-IGF2-R61A treatment, she is to be informed of the potential hazards to the fetus. In some embodiments, the label comprises instruction for use in adults. In some embodiments, the instruction for use in adults indicates that adults aged 18 years or older may be eligible to receive the treatment. In some embodiments, the instruction for use in adults indicates that adults aged 20 years or older may be eligible to receive the treatment. In some embodiments, the label comprises instruction for pediatric use. In some embodiments, the label indicates that HSA-IGF2-R61A is not to be administered to a subject less than 18 years of age. In some embodiments, the label comprises instruction for geriatric use. In some embodiments, the label indicates that HSA-IGF2-R61A is not to be administered to a subject that is over age 60. In some embodiments, the label indicates that HSA-IGF2-R61A administration is not recommended to a subject that is over age 60. In some embodiments, the label comprises information relating to overdosage. In some embodiments, the information relating to overdosage indicates that in case of overdose, it is recommended that the patient be monitored for any signs and symptoms of adverse reactions for effects and appropriate treatment be instituted immediately. In some embodiments, the label comprises a summary of clinical studies. In some embodiments, the label comprises storage conditions and information regarding how HSA-IGF2-R61A is supplied. In some embodiments, HSA-IGF2-R61A is a recombinant fusion protein (biologic) composed of HSA linked to a human IGF-2 sequence (HSA-IGF-2-R61A). In some embodiments, the label indicates that the formulation includes HSA-IGF2-R61A in 7 mM Sodium Citrate buffer, 8% sucrose (w/v) and 0.1% Pluronic 188 (w/v) at pH 6.0. Vials are to be stored at −20° C. In some embodiments, instructions for use indicate that resent shelf-life is for the product is 12 months.

In some embodiments, the label further comprises a summary of nonclinical study information. In some embodiments, the summary of nonclinical study information indicates primary pharmacology, safety pharmacology, pharmacokinetics, and/or toxicology. In some embodiments, the summary of nonclinical study information indicates that in the panCUG₉₆₀/+ murine DM1 model and in an aged mouse (sarcopenia) model showed that administration of HSA-IGF2-R61A was associated with improvements in skeletal muscle myopathology in the animals. Notably, administration of HSA-IGF2-R61A was associated with reversal of skeletal muscle mass loss, re-conversion of slow-twitch muscle fiber types to fast-twitch fiber types and improvements in functional assessments (grip strength and treadmill performance), observations that are directly relevant to patients with DM1. In some embodiments, the summary of nonclinical study information indicates that no stand-alone safety pharmacology studies have been conducted with HSA-IGF2-R61A. In some embodiments, the summary of nonclinical study information indicates that no HSA-IGF2-R61A-related effects on respiratory rates, neurological examinations or electrocardiogram (ECG) parameters (qualitative or quantitative). In some embodiments, the summary of nonclinical study information indicates that PK properties of HSA-IGF2-R61A administration were linear and dose-proportional in fed (SC) and fasted (IV) Sprague Dawley rats. In some embodiments, the summary of nonclinical study information indicates that IV administration of HSA-IGF2-R61A was associated with a dose-dependent decrease in blood glucose and that this effect was not seen with SC administration of HSA-IGF2-R61A up to 30 mg/kg. In some embodiments, the summary of nonclinical study information indicates that for toxicology studies, HSA-IGF2-R61A was administered subcutaneously (SC) Q3D for 4 weeks followed by a 4-week recovery period. TK and PD markers were evaluated. The high dose level of 60 mg/kg/dose was selected due to poor local tolerability of the 120 mg/kg/dose level in a prior dose-range-finding (DRF) study. In some embodiments, the summary of nonclinical study information indicates that no notable HSA-IGF2-R61A-related toxicity other than transient injection site-related findings were reported at any administered dose. In some embodiments, the summary of nonclinical study information indicates that a drastic drop in exposure over time was observed following HSA-IGF2-R61A administration. In some embodiments, the summary of nonclinical study information indicates that the No Observed Adverse Effect Level (NOAEL) was determined to be 60 mg/kg/dose under fed conditions in rats. In some embodiments, the summary of nonclinical study information indicates that toxicity observed at 20 mg/kg/dose in a dog included a single mortality associated with acute GI hemorrhage, transient minimal to mild findings in the kidney, transient injection site findings and hypersensitivity. In some embodiments, the label indicates that no serious adverse reactions (SARs) have been documented following administration of HSA-IGF2-R61A.

### EXAMPLES

The following examples are included for illustrative purposes only and are not intended to limit the scope of the invention.

### Example 1—an Inducible Myotonic Dystrophy 1 (DM1) Mouse Model Recapitulates DM1 Phenotype

A pan-inducible, TREDT960I transgenic mouse model containing a human genomic segment containing exons 11-15 of the human DMPK gene with 960 interrupted CTG repeats (CUG₉₆₀) under the direction of the tet-responsive elements (tetO) promoter was developed. TREDT960I mice were crossed with R26-M2rtTA mice to produce the inducible DM1 model CUG₉₆₀ mice. For induction of DM1 phenotype, CUG₉₆₀ mice (or panCUG₉₆₀/+) mice were fed 2 g/kg doxycycline for 9-10 weeks beginning at post-natal day 1 (PN1). Male and female mice were assayed separately. CUG₉₆₀ mice were determined to encompass key aspects of DM1 muscle deterioration as shown by both histological and functional testing. Distal muscle wasting and intracellular RNA foci accumulation in impacted tissues were both observed in adults in the animal model following transgene induction at PN1.

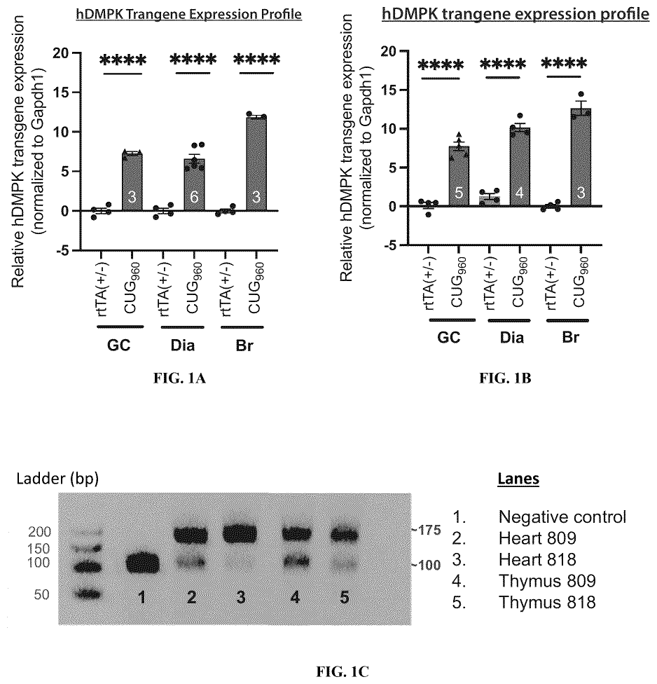

FIG. 1-FIG. 5 demonstrate the efficacy of induction and the DM1 symptomatology observed in adult CUG₉₆₀ mice. FIG. 1A-FIG. 1B demonstrate the extent of induction of human DM1 protein kinase (DMPK) transgene expression in male and female CUG₉₆₀ mice in various muscle tissues, normalized against GAPDH control expression levels. FIG. 1C shows induction of alternative splicing of Clcn1 mRNA transcripts in heart and thymus of CUG₉₆₀ mice.

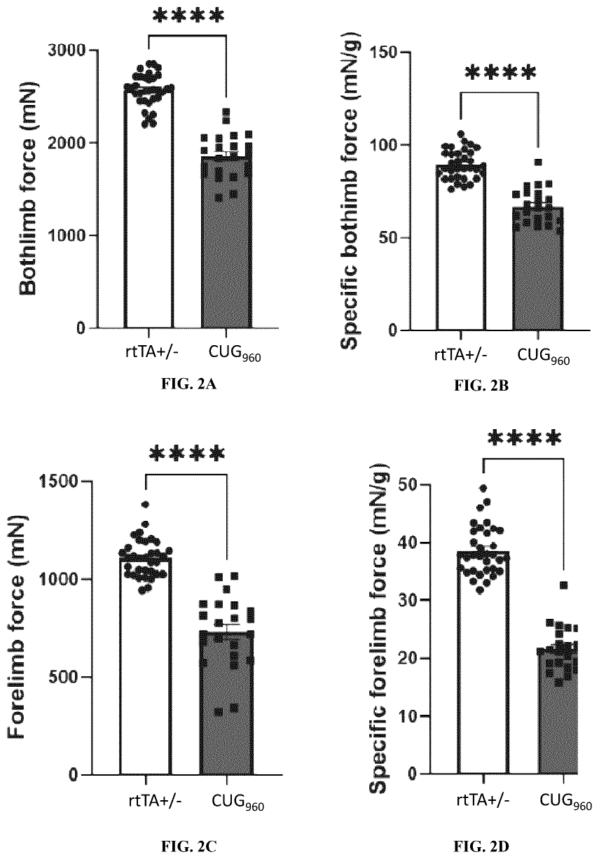

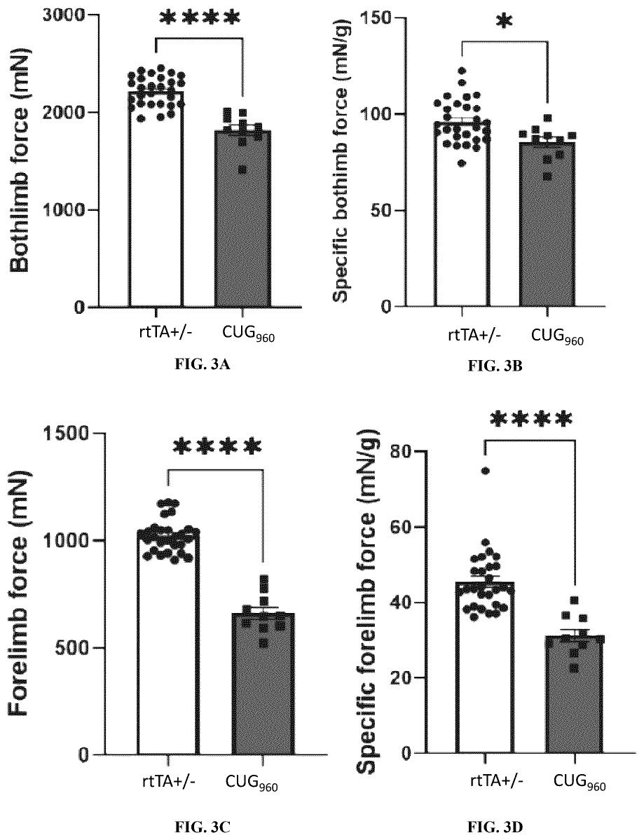

Grip strength and limb force was assayed in CUG₉₆₀ mice at 9-10 weeks of age. In FIG. 2A-FIG. 2D, the extent of muscle atrophy and grip strength reduction in males in the DM1 mouse model was determined (FIG. 2A: bothlimb force; FIG. 2B: specific bothlimb force; FIG. 2C: forelimb force; FIG. 2D: specific forelimb force). In FIG. 3A-FIG. 3D, the extent of muscle atrophy and grip strength reduction in females in the DM1 mouse model was determined (FIG. 3A: bothlimb force; FIG. 3B: specific bothlimb force; FIG. 3C: forelimb force; FIG. 3D: specific forelimb force).

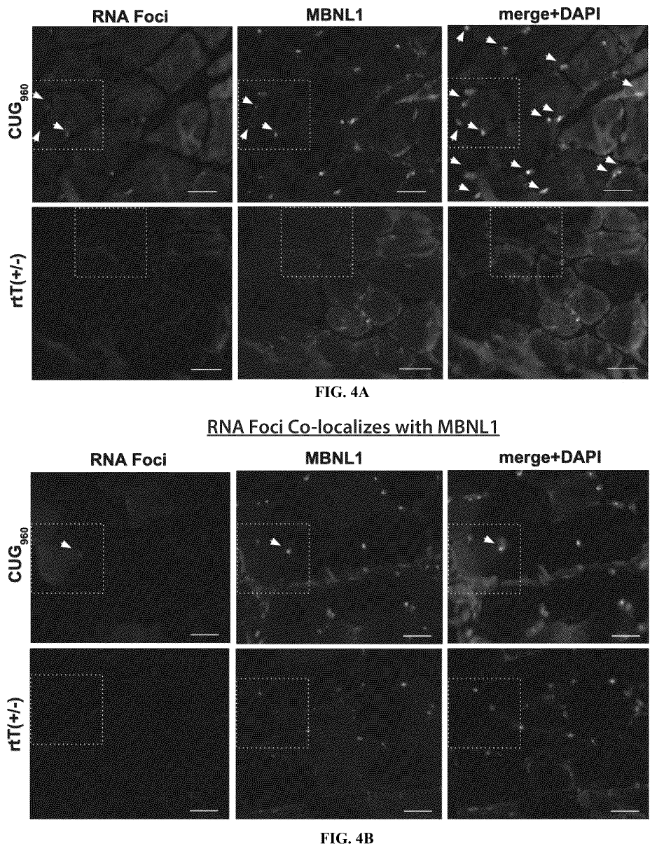

CUG₉₆₀ mice at 9-10 weeks of age were assayed for the presence of RNA foci and potential co-localization of RNA foci observed with MBNL1. FIG. 4A-FIG. 4B indicate the presence of RNA foci in muscle cells in CUG₉₆₀ male (in FIG. 4A) and female (in FIG. 4B) mice that co-localize with MBNL1. Control mice did not display evidence RNA foci.

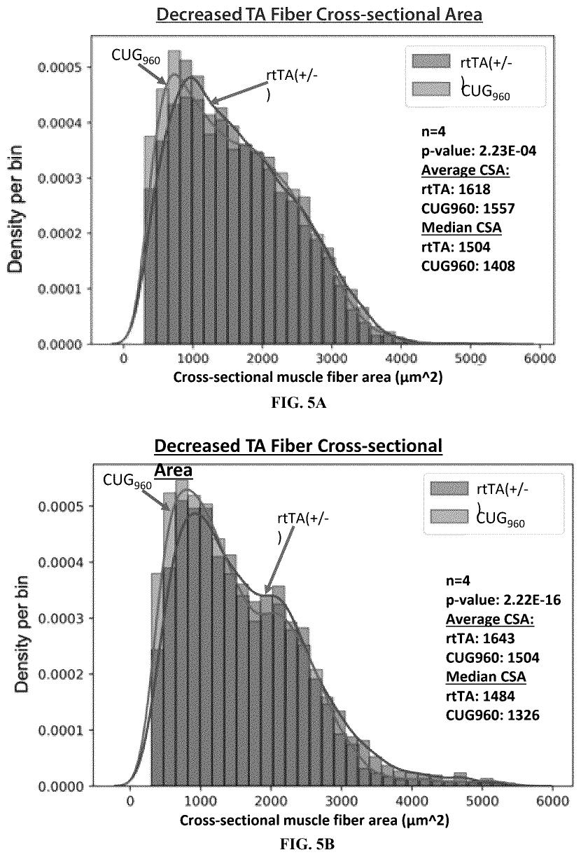

CUG₉₆₀ mice at 9-10 weeks of age were assayed for muscle atrophy as measure through laminin staining and calculation of TA fiber cross-sectional area (CSA). FIG. 5A-FIG. 5B demonstrate significant reductions in TA fiber CSA in both male (in FIG. 5A) and female (in FIG. 5B) CUG₉₆₀ mice.

The combination of mutant hDMPK gene expression, loss of grip strength, toxic RNA foci formation, subsequent aberrant mRNA splicing, and a decrease of CSA of TA muscle fibers confirmed that the CUG₉₆₀ mouse model represents DM1 pathology for both males and females.

### Example 2—In Vitro Treatment of Human Myoblasts with HSA-IGF2-R61A

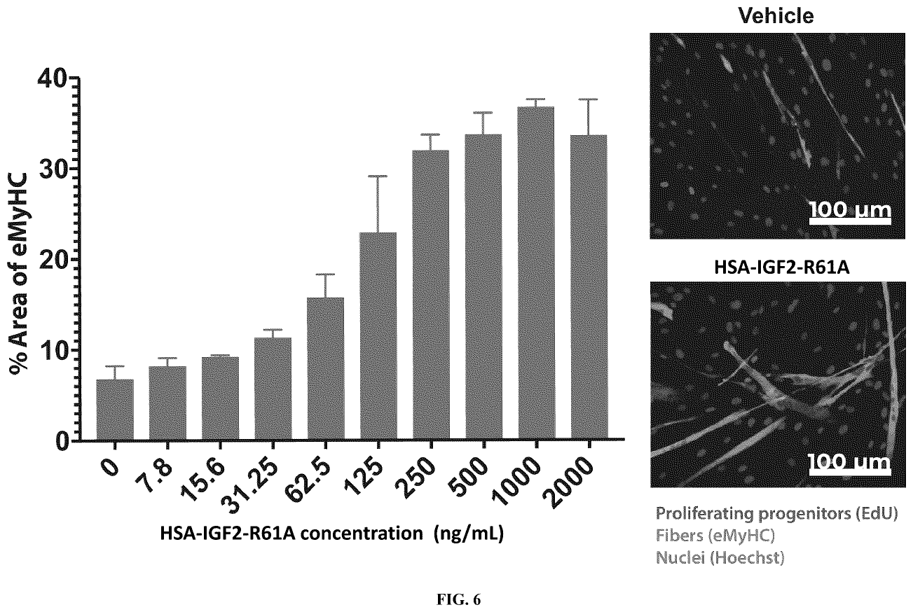

Human myoblasts from an age 30 female donor were cultured and plated at 20K cells/well and treated once every 24 hours for 72 hours with a range of concentrations of HSA-IGF2-R61A. After 72 hours, culture cells were stained for proliferation (EdU assay), to label nuclei (Hoechst), and to identity muscle fibers (eMyHC). Number of nuclei and proliferation rate were determined to be unaffected by HSA-IGF2-R61A treatment compared to controls. Treatment with HSA-IGF2-R61A lead to enhanced myogenesis via concentration-dependent human myoblast differentiation. FIG. 6 demonstrates the extent of concentration-dependent human myoblast differentiation.

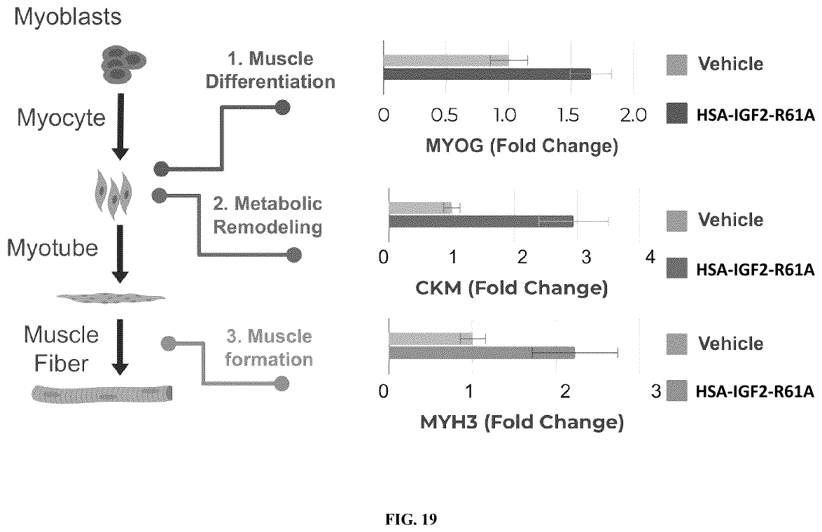

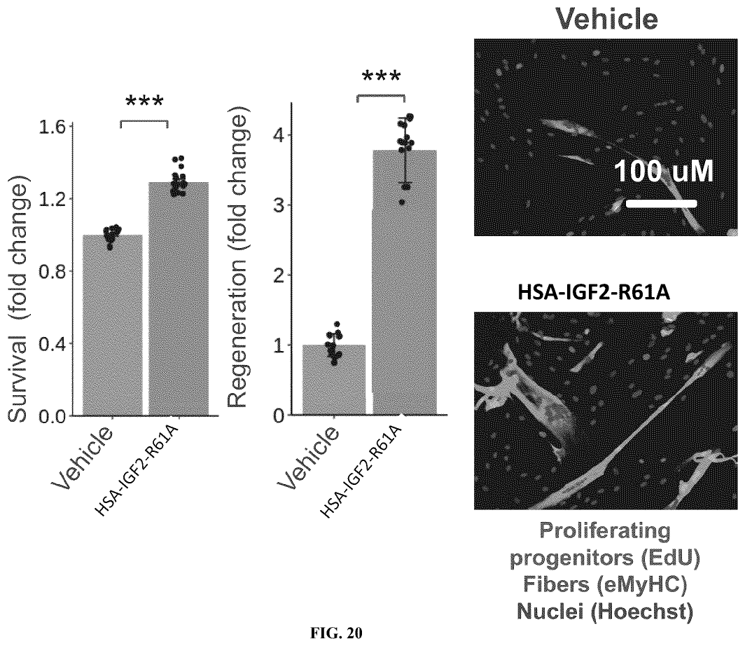

Human DM1 muscle precursors from 19-50 year-old male/female subjects diagnosed with DM1 were cultured and assayed for the effect of HSA-IGF2-R61A treatment on transcription factor level of key muscle regeneration transcription factors. Subjects were determined to have DMPK CTG repeats ranging between 202-394. Results in FIG. 19 indicate induction of MYOG, CKM, and MYH3 in treated cells as compared to expression levels in untreated cells. Cells were also assayed for survival and regeneration following HSA-IGF2-R61A treatment. Results in FIG. 20 indicate significant improvement in muscle cell survival and muscle regeneration following HSA-IGF2-R61A treatment in cultured DM1 cells as compared to untreated cultured DM1 cells. These results demonstrate the HSA-IGF2-R61A treatment is effective to improve myogenesis in muscle cells from DM1 subjects.

### Example 3—Administration of HSA-IGF2-R61A Restores Muscle Function, Fiber Size, and Fiber Composition in a Myotonic Dystrophy 1 (DM1) Mouse Model

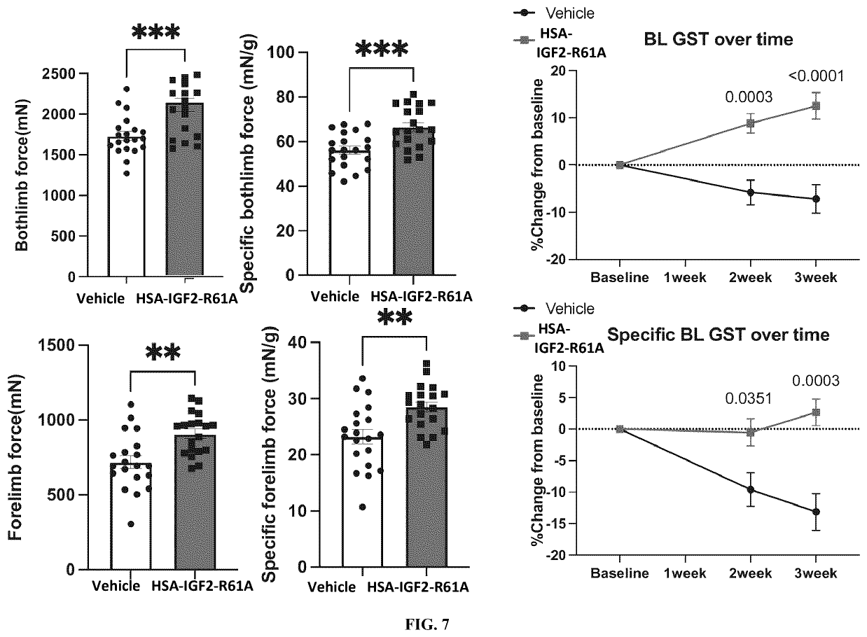

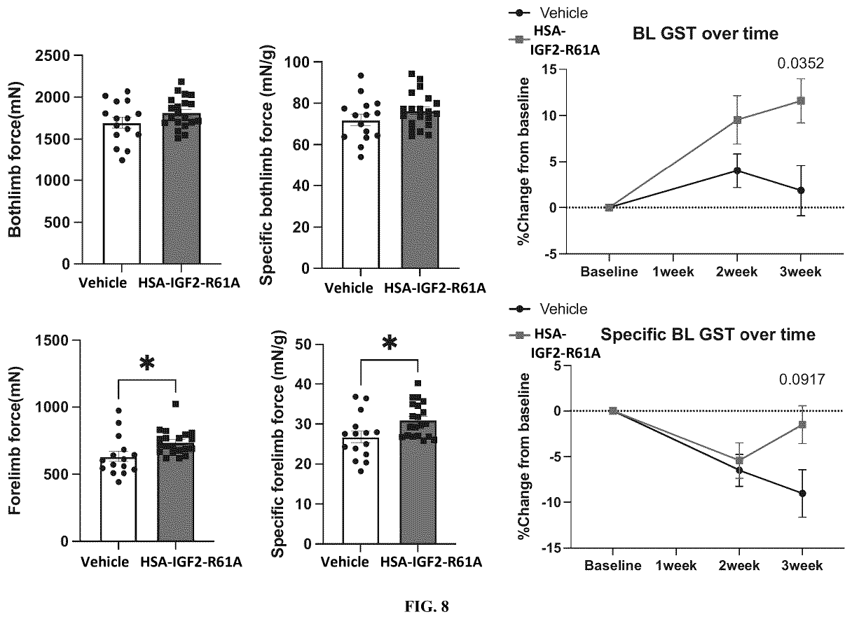

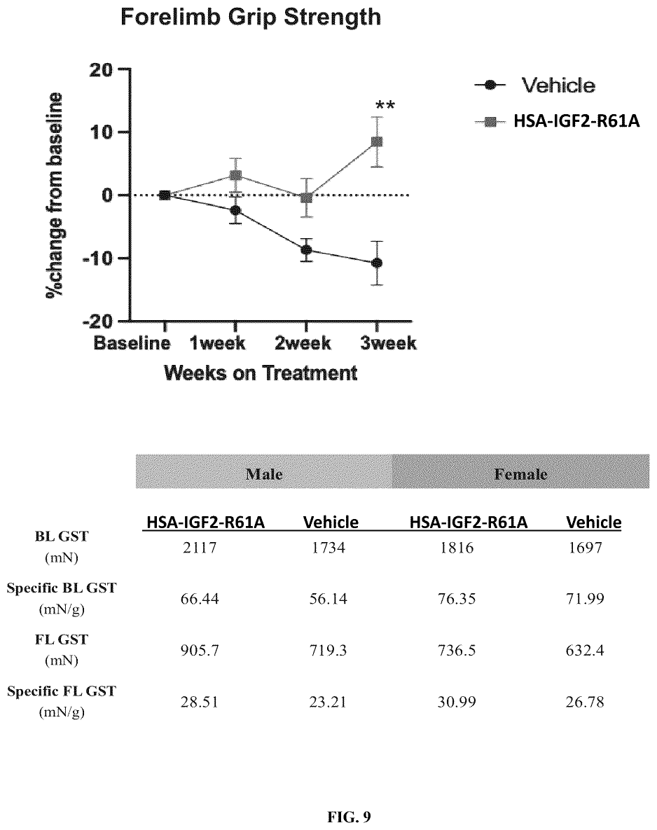

Male and female CUG₉₆₀ mice as described in Example 1, were treated with HSA-IGF2-R61A Q.O.D. for 3 weeks. PanCUG₉₆₀/+ mice were fed on 2 g/kg doxycycline beginning on PN1 for 9-10 weeks and through study duration. Mice were treated Q.O.D. with either 12 mg/kg body weight HSA-IGF2-R61A or PBS administered subcutaneously. FIG. 7-FIG. 8 display the results of grip strength tests following 3 weeks of treatment in males (in FIG. 7) and females (in FIG. 8) respectively. Bothlimb and specific bothlimb grip strength were additionally assayed at Week 0 and Week 2 to illustrate changes in effect on grip strength over the treatment period. Both male and female PanCUG₉₆₀/+ mice displays a protection of grip strength in HSA-IGF2-R61A-treated animals. Bothlimb grip strength improved over the period of treatment in both male and female PanCUG₉₆₀/+ mice. As shown in FIG. 9, forelimb grip strength improved over the period of treatment for male PanCUG₉₆₀/+ mice. A summary of effects of HSA-IGF2-R61A treatment on grip strength in male and female PanCUG₉₆₀/+ mice is provided below in Table 5.

TABLE 5
GST results in male and female PanCUG₉₆₀/+
mice following HSA-IGF2-R61A treatment

| Grip Strength Test (GST) | Sex | % increase vs. vehicle |
|---|---|---|
| Bothlimb GST | Male | 22% |
| Specific Bothlimb GST | Male | 18% |
| Forelimb GST | Male | 26% |
| Specific Forelimb GST | Male | 23% |
| Bothlimb GST | Female | 7% |
| Specific Bothlimb GST | Female | 6% |
| Forelimb GST | Female | 16% |
| Specific Forelimb GST | Female | 16% |

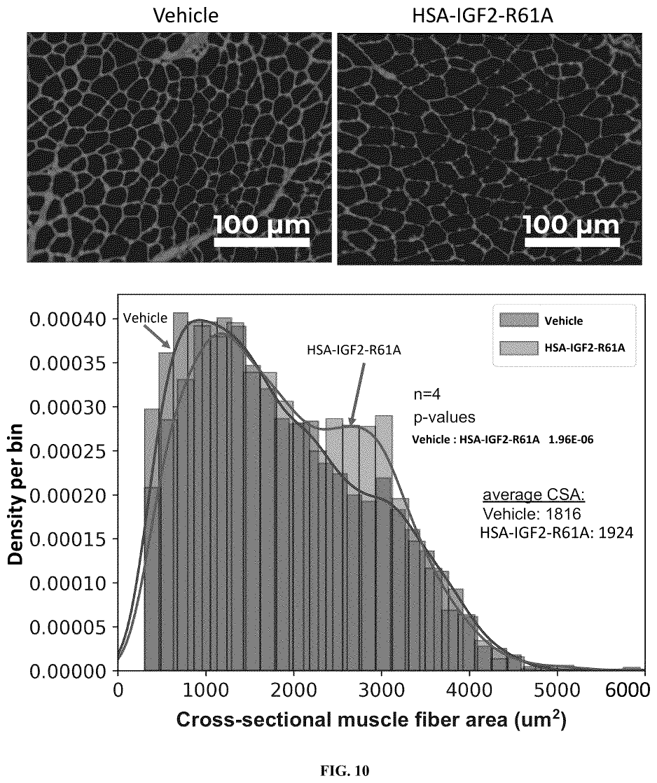

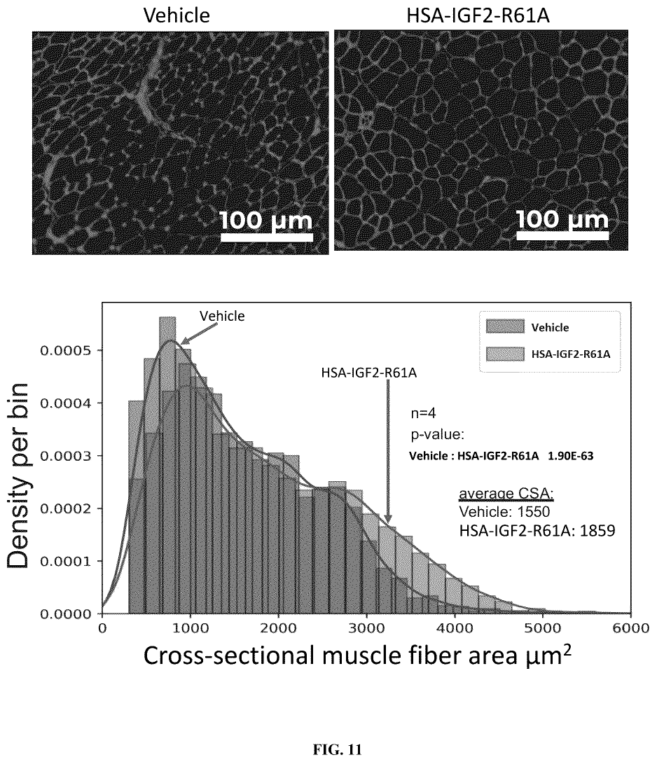

As shown in FIG. 10-FIG. 11, TA muscle fiber CSA was significantly increased in both male (in FIG. 10) and female (in FIG. 11) PanCUG₉₆₀/+ mice following the period of treatment.

Administration of HSA-IGF2-R61A in PanCUG₉₆₀/+ mice resulted in significant improvements in grip strength in both male and female mice indication effectiveness for prevent progression of DM1 symptoms and improvement of DM1 symptoms in this mouse model.

### Example 4—Pharmacokinetic and Pharmacodynamic Analysis of HSA-IGF2-R61A Administration in Mice, Rats, and Dogs

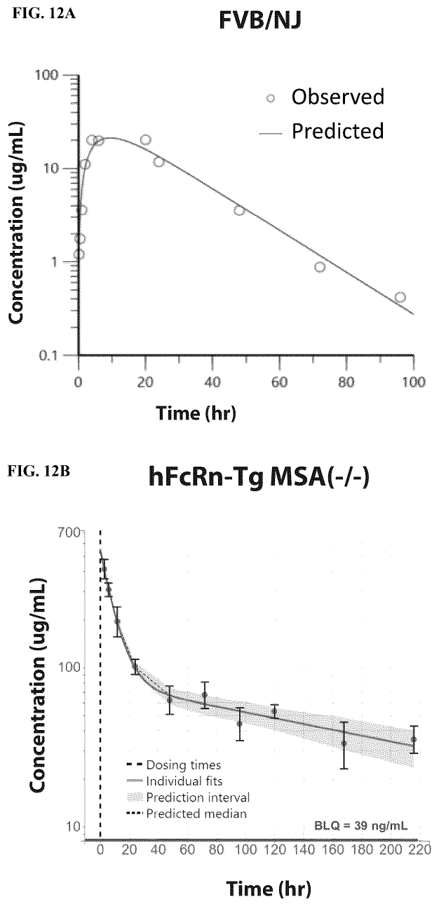

As shown in FIG. 12A, adult FVB/NJ mice were dosed subcutaneously with 12 mg/kg body weight of HSA-IGF2-R61A. Plasma concentrations of HSA-IGF2-R61A at various time points were assayed and plotted following the administration. Results were analyzed using Certara Phoenix WinNonLin software and fitted to a one-compartment first order model.

As shown in FIG. 12B, adult hFcRn-Tg MSA(−/−) mice were dosed intravenously with 161 μg of HSA-IGF2-R61A. Plasma concentrations of HSA-IGF2-R61A at various time points were assayed and plotted following the administration. Results were analyzed using Certara Phoenix WinNonLin software and fitted to a non-compartment model. Serum half-life was determined to by >136.5 hours.

These results demonstrate that administration of HSA-IGF2-R61A via s.c. or i.v. administration produce predictable PK profiles enabling extended bioavailability of HSA-IGF2-R61A to cell types following administration.

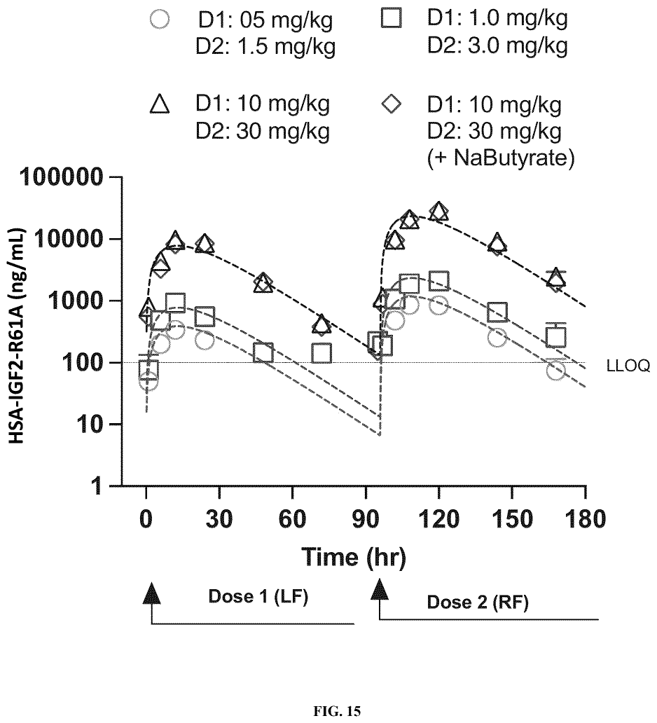

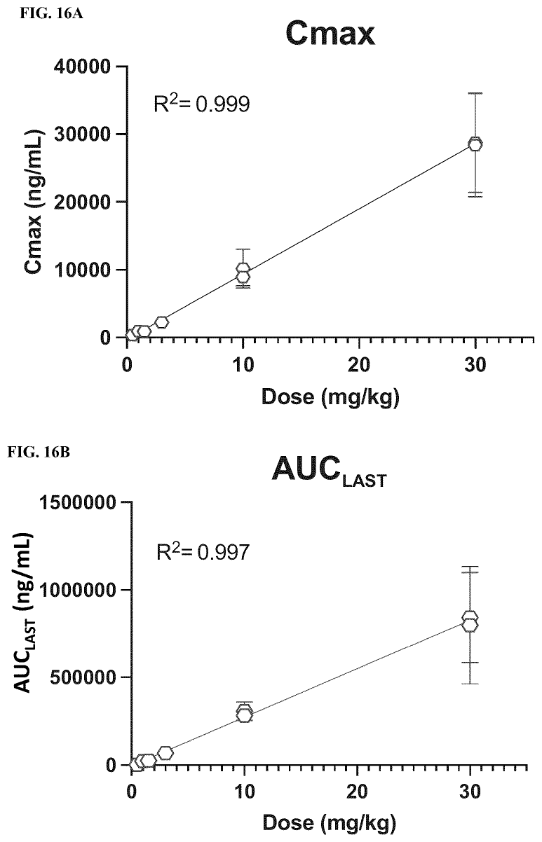

As shown in FIG. 15, adult rats were dosed subcutaneously with various concentrations of HSA-IGF2-R61A and the resulting PK profiles were analyzed. A first dose was administered s.c. at time zero and a second dose was administered s.c. 96 hours following the first dose. A one-compartment model closely fit the data following the first dose allowing the PK profile predictions following the second dose to be estimated. Cₘₐₓ and AUC_{LAST} were calculated in FIG. 16A and FIG. 16B respectively.

PK profiles of HSA-IGF2-R61A were assessed over a wide dose range (to assess linearity in PK in rats and dogs). PK profiles of HSA-IGF2-R61A were assessed following multiple dose administration and to establish the stationary nature (lack of time-dependency in rats and dogs) of the PK over time. Injection site reaction was monitored following single and multiple SC dose administration. The effect of sodium butyrate, as an excipient, on the absorption profile of HSA-IGF2-R61A was assessed following SC dose administration. In addition, MTD studies in Sprague Dawley rats and Beagle dogs were conducted to evaluate the PK and PD relationship between exposure and potential hypoglycemic effect following IV (Rats and Dogs) and SC (Dogs) administration of HSA-IGF2-R61A in a dose escalation fashion under FASTED conditions. Results obtained from these studies indicated dose-proportional (linear PK) and time-independent (stationary) PK properties for HSA-GF2-R61A in rats and dogs following single and multiple dose administration. Additionally, under the FED condition, no impact on blood glucose, or signs of hypoglycemia were observed. Absolute bioavailability of HSA-IGF2-R61A in dogs following SC administration was complete (108). Table 6 summarizes nonclinical PK and PD studies in mice, rats, and dogs for administering HSA-IGF2-R61A.

TABLE 6
Summary of the Nonclinical PK and PD Studies in Mice, Rats and Dogs

| Study ID | | Study Objectives | |
|---|---|---|---|
| (CRO) | Study Title | (non-GLP/Research) | Study Results |
| MS072 PK IV | Pharmacokinetics | To determine the PK | In naïve male FVB/NJ mice, |
| and SC (Juvena | of HSA- | of a single dose of | administration of a single 12 mg/kg SC |
| Therapeutics) | IGF2-R61A | HSA-IGF2-R61A | dose of HSA-IGF2-R61A resulted in a |
| | Following | following SC or IV | rapid absorption profile from the SC |
| | Single-dose | administration in | space into systemic circulation. The |
| | Subcutaneous | FED, male naïve | comparison of the PK profiles following |
| | or Intravenous | mice | SC and IV dosing demonstrated no |
| | Administration | To predict the PK of | evidence of flip-flop kinetics. |
| | to Naïve | HSA-IGF2-R61A | Simulations showed that concentrations of |
| | Fvb/Nj Mice | dosed SC QOD to | HSA-IGF2-R61A in serum should |
| | Under | guide dosing | provide adequate exposure in efficacy |
| | Nonfasted | regimens for | studies where the comparable dosing |
| | Conditions | nonclinical | paradigm is employed (i.e., 12 mg/kg |
| | | pharmacology | QOD). |
| | | studies | |
| Rat_JUV_2022_PKPD | PK and PD | To determine the PK | PK properties of HSA-IGF2-R61A |
| (WuXiAppTec) | Study of HSA- | and PD of HSA- | following SC administration over a wide |
| | IGF2-R61A In | IGF2-R61A | dose range (at 0.5, 1.0 to 10 mg/kg) on |
| | Male SD Rats | following SC | the study Day 1 were linear and dose- |
| | following | administration to | proportional. |
| | Multiple | FED male SD rats | Administration of HSA-IGF2-R61A (at |
| | Subcutaneous | following dose | 1.5, 3.0, 30 mg/kg) on the study Day 4 |
| | Administration | administration on | was predictable from the exposure-time |
| | (FED) | study Days 1 and 4. | data following administration of the dose |
| | | To assess injection | on the study Day 1. |
| | | site reaction | These results indicated time-independent |
| | | following SC dosing | Pproperties in rats following SC |
| | | of HSA-IGF2-R61A +/− | administration of the second dose |
| | | sodium butyrate | Sodium butyrate, an excipient, had no |
| | | To evaluate the effect | effect on the absorption of HSA-IGF2- |
| | | of sodium butyrate as | R61A PK. |
| | | an excipient on the | No injection site reaction was observed in |
| | | absorption profile of | rats following administration of HSA- |
| | | HSA-IGF2-R61A | IGF2-R61A throughout the study. |
| | | following SC dose | Under the FED condition, no impact on |
| | | administration | blood glucose, or signs of hypoglycemia |
| | | | was observed. |
| Rat_JUV_2023_MTD_01 | An Exploratory | To determine the | Results from this MTD study are |
| (WuXiAppTec) | Dose | Maximum Tolerated | significant to support rats as a relevant |
| | Escalation | Dose (MTD) of HSA- | species for the conduct of the |
| | MTD Study of | IGF2-R61A | pharmacology/toxicology studies. |
| | HSA-IGF2- | following daily dose | HSA-IGF2-R61A serum exposure |
| | R61A in Male | escalation of IV doses | following IV administration in FASTED |
| | and Female SD | in male and female | rats appeared linear and dose- |
| | Rats following | SD rats | proportional. |
| | Daily | | Under the FASTED conditions, a dose- |
| | Intravenous | | dependent decrease in blood glucose was |
| | Administration | | observed. |
| | (FASTED) | | Approximately a 40% decrease in |
| | | | blood glucose was observed 2 hours |
| | | | after IV administration of HSA- |
| | | | IGF2-R61A at 10 mg/kg under |
| | | | FASTED conditions. |
| | | | The decrease in blood glucose at t = 2 |
| | | | hours was more pronounced for the |
| | | | 30 mg/kg and 100 mg/kg dose (~55% |
| | | | and >70%, respectively). |
| | | | Results from this study indicated that the |
| | | | hypoglycemic effect is observed in |
| | | | FASTED animals following IV dosing. |
| | | | The hypoglycemic effects were not |
| | | | observed in FED animals following SC |
| | | | administration of HSA-IGF2-R61A at |
| | | | doses up to 30 mg/kg previously (Study: |
| | | | Rat_JUV_2022_PKPD). |
| Dog_JUV_2022_PKPD_01 | PK and PD | To determine the PK | PK properties of HSA-IGF2-R61A |
| (WuXiAppTec) | Study of HSA- | and PD of HSA- | following IV and SC administration in a |
| | IGF2-R61A in | IGF2-R61A | stepwise fashion appeared linear and |
| | Male Beagle | following IV infusion | dose-proportional |
| | Dogs | or SC administration | Administration of HSA-IGF2-R61A in a |
| | Following | of HSA-IGF2-R61A | stepwise fashion in dogs (1- animal per |
| | Intravenous | to FED male beagle | arm) reflected a predictable exposure-time |
| | Infusion and | dogs in a stepwise | profile |
| | Subcutaneous | dose-escalation | These results support the stationery and |
| | Administration: | fashion | time-independent nature of the PK |
| | Stepwise | To assess the | properties in dogs following IV and SC |
| | Dose | injection site reaction | administration following multiple dosing |
| | Escalation | following SC dosing | Sodium butyrate, an excipient, had no |
| | Design (FED) | of HSA-IGF2-R61A +/− | effect on the absorption of HSA-IGF2- |
| | | sodium butyrate | R61A PK |
| | | To evaluate the effect | No injection site reactions were observed |
| | | of sodium butyrate as | in dogs following administration of HSA- |
| | | an excipient on the | IGF2-R61A |
| | | absorption profile of | Under the FED condition, no impact on |
| | | HSA-IGF2-R61A | blood glucose, or signs of hypoglycemia |
| | | following SC dose | were observed |
| | | administration | |
| Dog_JUV_2023_MTD_01 | An Exploratory | To determine the | HSA-IGF2-R61A serum exposure |
| (WuXiAppTec) | Dose | MTD of HSA-IGF2- | following IV administration in FASTED |
| | Escalation | R61A following daily | dogs appeared linear. |
| | MTD Study of | dose escalation of IV | Under the FASTED conditions, the drop |
| | HSA-IGF2- | and SC dose in male | in the blood glucose was more |
| | R61A In Male | and female beagle | pronounced following IV dosing as |
| | and Female | dogs | compared to SC administration. |
| | Beagle Dogs | | The hypoglycemic effects were not |
| | Following | | observed in FED animals following SC |
| | Daily | | administration of HSA-IGF2-R61A |
| | Intravenous & | | |
| | Subcutaneous | | |
| | Dose | | |
| | Administration | | |
| | (FASTED) | | |
| Dog_JUV_2023_PKPD_02 | Pharmacokinetic | To determine the | All animals tolerated HSA-IGF2-R61A |
| (WuXiAppTec) | Evaluation of | pharmacokinetics | during the entire course of the study. |
| | HSA-IGF2- | (PK) of HSA-IGF2- | No adverse effect was observed during |
| | R61A in Naive | R61A in serum, | the in-life phase of the study. |
| | Dogs following | following intravenous | Absolute Bioavailability following SC |
| | IV and SC | (IV) bolus or | administration was complete. |
| | Dosing in FED | subcutaneous (SC) | |
| | Animals | administration of | |
| | | HSA-IGF2-R61A to | |
| | | FED male naive | |
| | | beagle dogs | |

Abbreviations:
IV: Intravenous;
MTD: Maximum tolerated dose;
PD: pharmacodynamics;
PK: pharmacokinetics;
SC: Subcutaneous

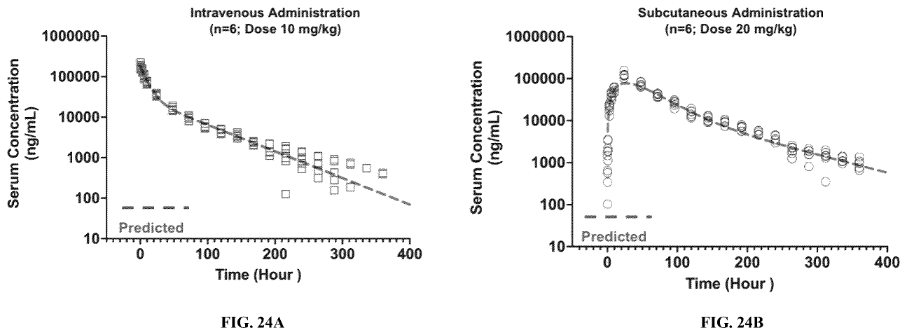

Further in this Example, a pharmacokinetic HSA-IGF2-R61A valuation of HSA-IGF2-R61A in naive dogs following IV And SC dosing in FED animals, (Study Dog_JUV_2023_PKPD_02, Non-GLP) was conducted. The goal of this study was to determine the PK of HSA-IGF2-R61A in serum, following IV bolus or SC administration of HSA-IGF2-R61A to FED male naive beagle dogs and to assess HSA-IGF2-R61A absolute bioavailability and to provide high resolution PK data for predictions of human PK in healthy subjects. Twelve naïve male beagle dogs were divided into two groups with 6 animals/group. Animals in Group 1 were administered HSA-IGF2-R61A by single IV bolus administration at 10 mg/kg. Animals in Group 2 were administered HSA-IGF2-R61A by single SC administration at 20 mg/kg. For Groups 1 and 2, blood samples for glucose test and serum samples for PK were collected at pre-dose (0), 0.5, 1, 3, 6, 10, 24, 48, 72, 96, 120, 144, 168, 192, 216, 240, 264, 288, 312, 336, 360 and 600 hours post-dose. The serum concentration-time profiles for the individual animals in each group are shown in FIG. 24A-FIG. 24B (graph of IV administration in FIG. 24A; graph of subcutaneous administration in FIG. 24B). Pharmacokinetic parameters estimate is shown in Table 7.

TABLE 7
PK Parameters Estimate of HSA-IGF2-R61A in Dogs
Following IV and SC Dose Administration (Study: Dog_JUV_{—}
2023_PKPD_02; Noncompartmental Analysis)

| | Group | | | |
|---|---|---|---|---|
| | 1 (n = 6) | | 2 (n = 6) | |
| | Dose Route | | | |
| | IV Bolus | | SC | |
| | Dose Level (mg/kg) | | | |
| | 10 | | 20 | |
| PK Parameters | Mean | SD | Mean | SD |
| C₀ or Cₘₐₓ (ng/mL) | 206536 | 55260 | 122833 | 18335 |
| Tₘₐₓ (h) | — | — | 24.0 | 0.00 |
| T_{1/2} (h) | 46.2 | 16.7 | 61.7 | 26.6 |
| AUC_{Extra} (%) | 0.561 | 0.410 | 0.558 | 0.358 |
| Bioavailability (%)c | — | — | 111 | — |
| Bioavailability (%)d | — | — | 108 | — |

C₀ was reported for the IV route and Cₘₐₓ was reported for the SC route
b: Range
cAbsolute bioavailability was calculated using AUC_{0-inf} and nominal dose.
dBioavailability was calculated using AUC₀₋₂₀₀ and nominal dose.
“—” means not calculated.

In this study, all animals tolerated HSA-IGF2-R61A at the dosing levels during the entire course of the study. No adverse effect was observed during the in-life phase of the study. Additionally, administration of HSA-IGF2-R61A to FED male dogs at 10 mg/kg IV or 20 mg/kg SC did not produce a clear or statistically significant test article related effect on blood glucose. However, there was a trend toward reduced blood glucose at 3 to 10 hours post-dose when administered IV and 10 hours post dose when administered SC.

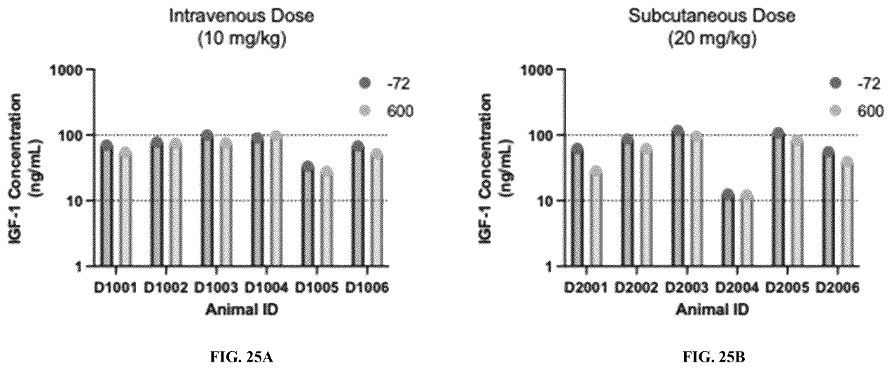

In some animals in both groups shown in FIG. 25A-FIG. 25B (chart of IV administration in FIG. 25A; chart of subcutaneous administration in FIG. 25B) the PK profiles appeared to deviate from the earlier reproducible and consistent profiles after the 200-hour sample. The deviations observed in exposure in these animals are consistent with the potential development of the anti-drug antibody following administration of the fully human protein HSA-IGF2-R61A following both SC and IV administration. Interestingly, no differences in the endogenous IGF levels collected at baseline (prior to HSA-IGF2-R61A administration) and 600 hours after HSA-IGF2-R61A was observed in either group in all animals (FIG. 25A-FIG. 25B). These results are significant as they reflect the absence of any correlation between the aberrant changes in HSA-IGF2-R61A serum concentrations (after 200-hour samples due to potential immunogenicity) and IGF concentrations post HSA-IGF2-R61A administration. In FIG. 25A-FIG. 25B, the endogenous IGF-1 concentrations at baseline (−72 Hours) and post HSA-IGF2-R61A administration (600 hours) in the individual animals in each dosing cohorts. Due to interference of HSA-IGF2-R61A with the IGF-2 assay endogenous IGF-2 concentrations could not be measured.

### Example 5—Concentration-Dependent Binding of HSA-IGF2-R61A to Primary Muscle Cells

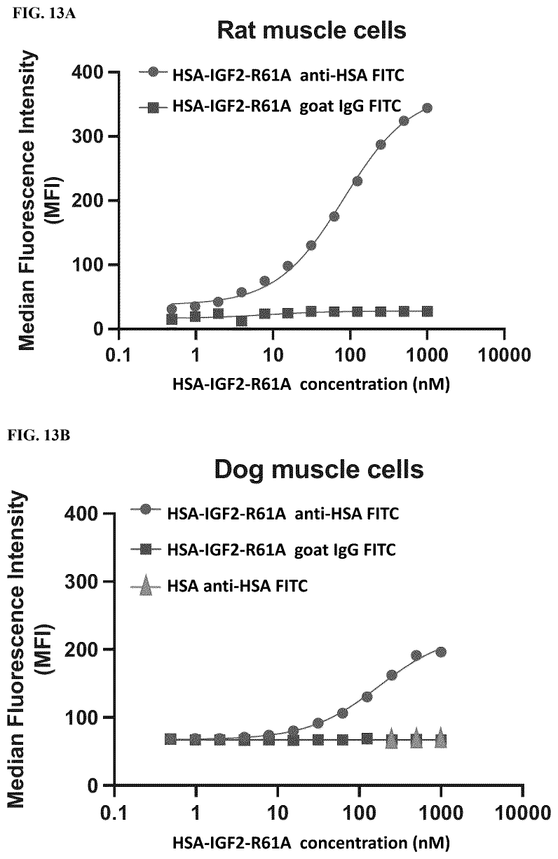

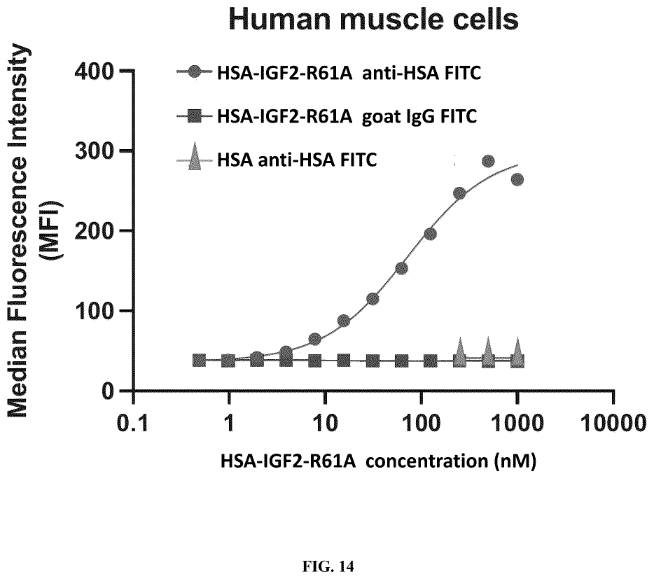

Rat, dog, and human primary muscle cells were obtained and cultured. Various concentrations of HSA-IGF2-R61A were added to the culture medium and specific binding of HSA-IGF2-R61A to primary muscle cells was assayed. As shown in FIG. 13A-FIG. 13B, FIG. 14, HSA-IGF2-R61A was determined to bind specifically to primary muscle cells from rat, dog, and human in a concentration-dependent manner. FIG. 13A shows specific binding in rat primary muscle cells. FIG. 13B shows specific binding in dog primary muscle cells. FIG. 14 shows specific binding in human primary muscle cells.

### Example 6—Administration of HSA-IGF2-R61A Protect Muscle Atrophy, Restores Muscle Composition, and Restores Metabolism in a Myotonic Dystrophy 1 (DM1) Mouse Model

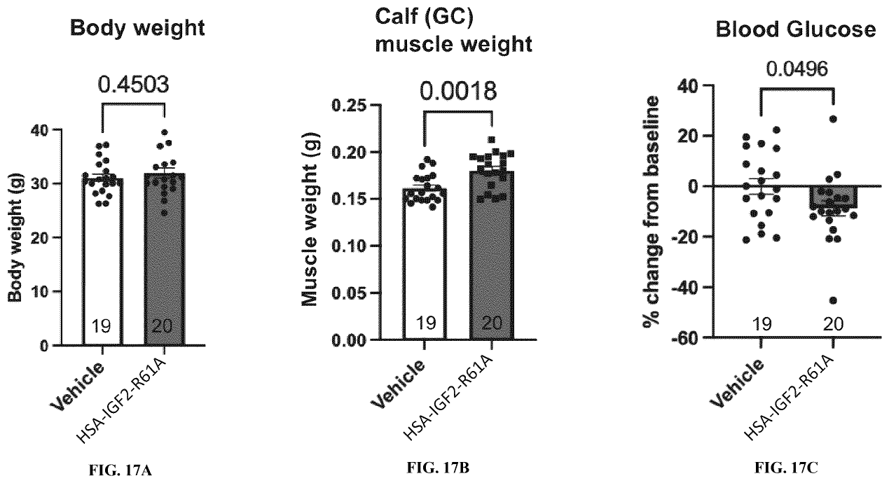

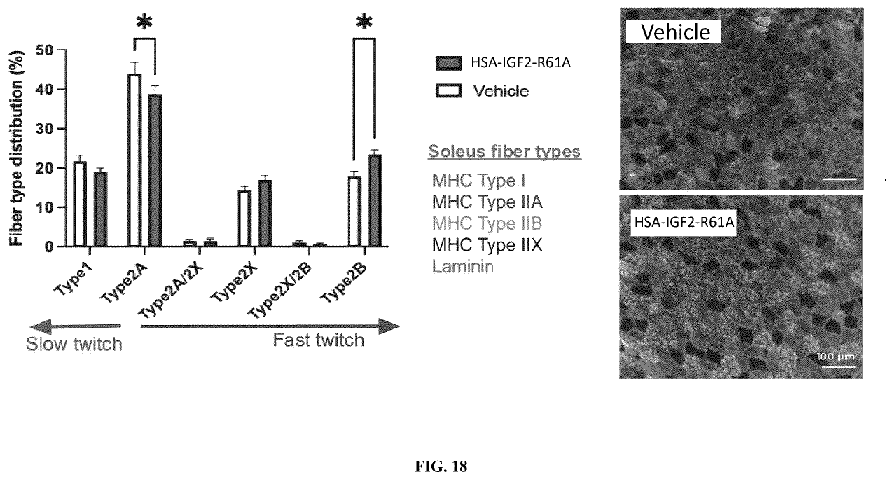

The DM1 mouse model described in Example 1 were utilized to determine the effect of HSA-IGF2-R61A treatment on body weight, muscle mass, and blood glucose. PanCUG₉₆₀/+ mice were fed on 2 g/kg doxycycline beginning on PN1 for 9-10 weeks and through study duration. Mice were treated Q.O.D. with either 12 mg/kg body weight HSA-IGF2-R61A or PBS administered subcutaneously. After 3 weeks of treatment, mice were assayed for body weight, muscle mass, and blood glucose. Results in FIG. 17A-FIG. 17C indicate that HSA-IGF2-R61A treatment did not significantly impact body weight but did have a significant effect on preserving muscle weight and in restoring blood glucose levels. FIG. 17A shows a graph of body weight with HSA-IGF2-R61A or vehicle treatment. FIG. 17B shows a graph of calf (GC) muscle weight with HSA-IGF2-R61A or vehicle treatment indicating that HSA-IGF2-R61A treatment significantly increased muscle weight. FIG. 17C shows a graph of blood glucose with HSA-IGF2-R61A or vehicle treatment indicating that HSA-IGF2-R61A treatment significantly improved blood glucose levels measured as a percentage decrease from baseline. Results in FIG. 18 indicate that HSA-IGF2-R61A treatment restored muscle composition by significantly reducing the measured distribution of Type2A muscle fibers and significantly increasing the measured distribution of Type2B muscle fibers.

### Example 7—Bioinformatic Analysis of HSA-IGF2-R61A Treatment of Differentiating Primary DM1 Human Myoblasts

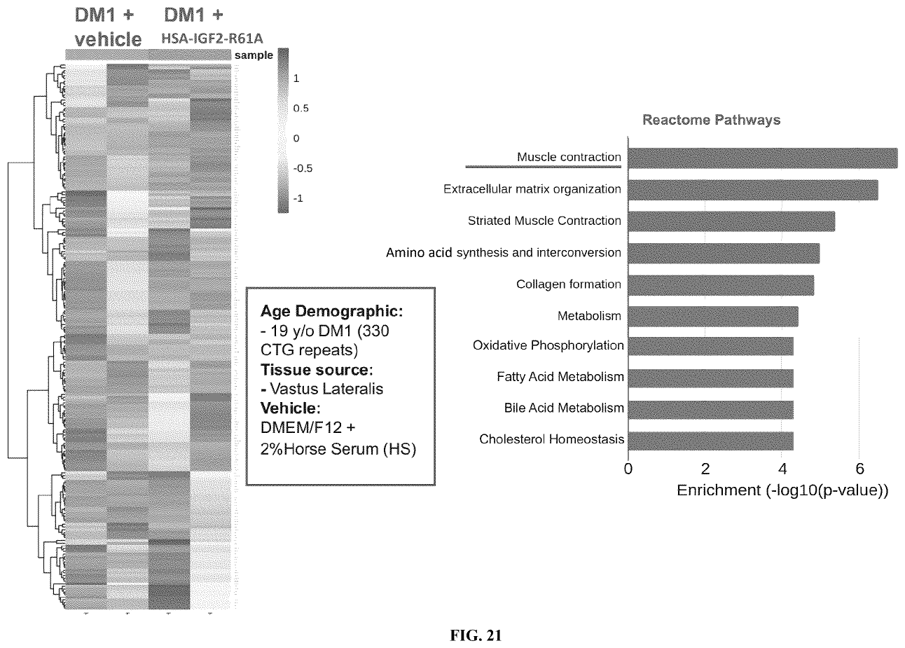

Primary human myoblasts derived from vastus lateralis from a subject diagnosed with DM1 (age 19 years; determined to have 330 DMPK CTG repeats) were cultured in DMEM/F12 media plus 2% horse serum. Cells were subjected to HSA-IGF2-R61A-treatment or vehicle treatment. Following treatment, RNA was extracted from treated cells and prepared for RNAseq analysis. Biological pathways indicating significant enrichment in transcription were plotted in FIG. 21. Noteworthy, several metabolic reactome pathways and other pathways involved in muscle contraction, extracellular matrix organization, and collagen formation were indicated as being enriched following HSA-IGF2-R61A-treatment. Pathway dysregulation involving PI3K/AKT, ERK1/2 signaling was confirmed and the effect of HSA-IGF2-R61A-treatment on this pathway dysregulation was assessed.

### Example 8—Toxicology Studies of Administering HSA-IGF2-R61A

In this Example, toxicology studies were conducted to assess any potential harmful effects of administering HSA-IGF2-R61A. Good laboratory practice (GLP) and dose range finding (DRF) toxicology studies were conducted to:
(i) Determine the potential toxicity of 2HSA-GF2-R61A when administered by SC injection once every three days for eighteen days (six doses in total) and once every three days for 4-weeks (ten doses in total), for the DRF and GLP studies, respectively; and
(ii) Evaluate the toxicokinetics (TK) of HSA-IGF2-R61A.
   Table 8 lists features of the toxicology studies.

TABLE 8
Summary of the Toxicology Studies

| Study ID (CRO) | Study Title | Study Objectives | Report Number (Study Status) |
|---|---|---|---|
| Rat_JUV_2023_DRF_01 | HSA-IGF2-R61A: 18- | To determine the potential toxicity of HSA- | Rat_JUV_DRF_Report_01 |
| (WuxiAppTec) | Day Repeated | IGF2-R61A when administered once every | (Completed) |
| | Subcutaneous Injection | three days by SC injection to Sprague- | |
| | Toxicity and | Dawley rats for 18 days (once every three | |
| | Toxicokinetic Study in | days, six doses in total) and to establish the | |
| | Rats | doses for the GLP study. | |
| | | To determine the TK of HSA-IGF2-R61A | |
| | | in rats. | |
| 20465144 | A 4-Week Study of | To determine the potential toxicity of HSA- | GLP-Tox-Rat-01 |
| (Charles River | HSA-IGF2-R61A by | IGF2-R61A when administered once every | (2023) |
| GLP) | Subcutaneous Injection | three days by SC injection to Sprague- | |
| | Administration in Rats | Dawley rats for 4-weeks (once every three | |
| | with a 4-Week | days, ten doses in total) and to determine | |
| | Recovery Period Under | reversibility of any findings. | |
| | FED Conditions | To determine the TK of HSA-IGF2-R61A. | |
| Dog_JUV_2023_DRF_01 | HSA-IGF2-R61A: 18- | To determine the potential toxicity of HSA- | Dog_JUV_DRF_Report_01 |
| (WuxiAppTec) | Day Repeated | IGF2-R61A when administered by SC | (Completed) |
| | Subcutaneous Injection | injection to Beagle Dog for 18 Days (once | |
| | Toxicity and | every three days, six doses in total) and to | |
| | Toxicokinetic Study in | establish the doses for the GLP study. | |
| | Beagle Dogs | To determine the TK of HSA-IGF2-R61A | |
| | | in dogs | |
| 20465145 | A 4-Week Study of | To determine the potential toxicity of HSA- | GLP-Tox-Dog-01 |
| (Charles River, | HSA-IGF2-R61A by | IGF2-R61A when administered by SC | (2023) |
| GLP) | Subcutaneous | injection to Beagle Dog for 4-weeks (once | |
| | Administration in Dog | every three days, ten doses in total) and to | |
| | with a 4-Week | determine reversibility of any findings. | |
| | Recovery Period | To determine the TK of HSA-IGF2-R61A | |
| | | in dogs | |

### Single Dose Studies:

No single dose toxicity studies have been conducted. Single dose tolerability was evaluated in PK and PD studies. HSA-IGF2-R61A was well tolerated at up to 30 mg/kg (SC) in the Sprague-Dawley rat and at up to 20 mg/kg (SC) in the beagle dog under FED conditions. FED conditions prevent the hypoglycemia observed in some pilot studies under fasted conditions.

Dose-Range Finding Study in Rats (Rat_JUV_2023_DRF_01; non-GLP; FED):

The study design is shown Table 9; the objective of the repeat-dose DRF study in rats is to determine the potential toxicity of HSA-IGF2-R61A when administered SC every three days for 18 days under FED conditions in order to enable dose selection for the GLP repeat-dose toxicity study. The FED conditions prevent the hypoglycemia observed in some pilot studies under fasted conditions. In addition, TK and PD markers were also evaluated. The doses for this study were based on the previous IV and SC MTD, and PK and PD studies adjusted for an estimated SC bioavailability of approximately 50%.

TABLE 9
Sprague Dawley Rat Dose Range Finding Study Design (Study:
RAT_JUV_2023_DRF_01), Non-GLP

| | | Dose | Dose | Main Study | Toxicokinetic |
|---|---|---|---|---|---|
| | | (mg/kg/ | Volume | Animals | Animals |
| Group | Treatment | dose)^{a} | (mL/kg) | (M/F) | (M/F) |
| 1 | PBS | 0 | 3.0 | 3/3 | — |
| 2 | HSA-IGF2- | 6 | 0.15 | 5/5 | 6/6 |
| | R61A | | | | |
| 3 | HSA-IGF2- | 20 | 0.5 | 5/5 | 6/6 |
| | R61A | | | | |
| 4 | HSA-IGF2- | 60 | 1.5 | 5/5 | 6/6 |
| | R61A | | | | |
| 5 | HSA-IGF2- | 120 | 3.0 | 5/5 | 6/6 |
| | R61A | | | | |

^{a}HSA-IGF2-R61A was administered at a fixed concentration of 40 mg/mL.

Test-article related effects on standard toxicological endpoints including clinical observations, post-dose observations, injection site observations, body weights, food consumption, hematology, clinical chemistry, coagulation, organ weights, macroscopic and limited microscopic histopathology were evaluated. In addition to the standard parameters, effects on blood glucose levels, a PD marker, were monitored using a ONETOUCH Ultra Vue Glucometer at multiple timepoints on each dosing occasion. Samples for TK were collected for evaluation throughout the study.

All animals survived until the scheduled necropsy day. There were no test-article-related changes in clinical observations, body weight, food consumption, blood glucose, clinical pathology parameters, or organ weight.

Test-article-related macroscopic changes were focal or multifocal, SC red discoloration in the injection site(s) for both sexes at ≥60 mg/kg/dose.

Test-article-related microscopic changes consisted of minimal to moderate, focal or locally extensive, SC hemorrhage for both sexes at ≥60 mg/kg/dose, minimal to moderate, multifocal, SC, subacute inflammation for both sexes at ≥60 mg/kg/dose; and minimal to mild, focal or multifocal, SC mononuclear infiltration for males at 6, 20, and 60 mg/kg/dose and females at ≥6 mg/kg/dose in the injection site(s). Due to the inflammation that accompanied the hemorrhage, these changes at ≥60 mg/kg/dose in the injection site(s) were considered adverse.

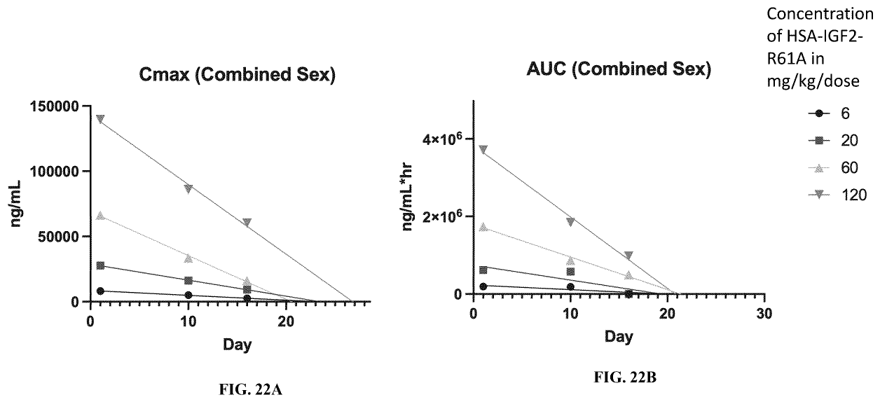

Toxicokinetic results after SC injection of HSA-IGF2-R61A at 6, 20, 60, or 120 mg/kg/dose to male and female rats once every 3 days for 18 days are shown in Table 10. It is notable that a drastic decline in exposure was observed (as seen in FIG. 22A-FIG. 22B) Day1>Day 10>Day 16), likely due to the presence of anti-drug antibodies (ADAs). No marked sex difference in systemic exposure was observed on Day 1. The systemic exposure increased dose proportionally in both sexes on Day 1. Data from Day 10 and Day 16 are confounded by the probable presence of ADAs. FIG. 22A shows a graph of the areas under the curve of the drug (AUC) (combined sex) when administered at 6, 20, 60, and 120 mg/kg/dose once every 3 days for 18 days by SC injection under FED conditions. FIG. 22B shows a graph of highest concentration of the drug (AUC) (combined sex) when administered at 6, 20, 60, and 120 mg/kg/dose once every 3 days for 18 days by SC injection under FED conditions.

TABLE 10
Selected TK Parameters in Rats Following Repeat Dose HSA-IGF2-R61A SC
Administration to FED Rats (Study: RAT_JUV_2023_DRF_01), Non-GLP

| Dose | Study | | Cₘₐₓ | Tmax | AUCτ | AUCτ/D |
|---|---|---|---|---|---|---|
| (mg/kg/dose) | Day | Sex | (ng/mL) | (h) | (h * ng/mL) | (h * kg * ng/mL/mg) |
| 6 | 1 | Male | 6700 | 12.0 | 167,000 | 27,900 |
| | | Female | 9710 | 12.0 | 218,000 | 36,400 |
| | 10 | Male | 3980 | 12.0 | 142,000 | 23,700 |
| | | Female | 6140 | 12.0 | 231,000 | 38,400 |
| | 16 | Male | 1650 | 12.0 | NA | NA |
| | | Female | 3510 | 12.0 | 134,000 | 22,400 |
| 20 | 1 | Male | 19,800 | 12.0 | 481,000 | 24,100 |
| | | Female | 35,600 | 12.0 | 757,000 | 37,800 |
| | 10 | Male | 13,500 | 12.0 | 501,000 | 25,100 |
| | | Female | 18,900 | 12.0 | 651,000 | 32,600 |
| | 16 | Male | 7720 | 12.0 | 267,000 | 13,300 |
| | | Female | 11,000 | 12.0 | NA | NA |
| 60 | 1 | Male | 59,600 | 12.0 | 1,580,000 | 26,400 |
| | | Female | 73,400 | 12.0 | 1,910,000 | 31,800 |
| | 10 | Male | 30,100 | 12.0 | 632,000 | 10,500 |
| | | Female | 37,300 | 12.0 | 1,310,000 | 21,900 |
| | 16 | Male | 22,200 | 12.0 | 769,000 | 12,800 |
| | | Female | 10,600 | 6.0 | 238,000 | 3960 |
| 120 | 1 | Male | 92,000 | 12.0 | 2,800,000 | 23,400 |
| | | Female | 187,000 | 12.0 | 4,620,000 | 38,500 |
| | 10 | Male | 59,900 | 12.0 | 1,150,000 | 9590 |
| | | Female | 112,000 | 12.0 | 2,530,000 | 21,100 |
| | 16 | Male | 44,400 | 12.0 | 813,000 | 6780 |
| | | Female | 76,000 | 12.0 | 1,140,000 | 9480 |

NA: Not applicable due to less than 3 quantitative time points.

Results: HSA-IGF2-R61A was well tolerated systemically when administered at 6, 20, 60, and 120 mg/kg/dose once every 3 days for 18 days by SC injection under FED conditions. No changes to blood glucose were observed at any dose when administered under these conditions. Adverse HSA-IGF2-R61A-related macroscopic and microscopic changes were limited to injection site(s) for both sexes at ≥60 mg/kg/dose, while no test-article-related systemic adverse changes were observed. Therefore, the NOAEL for systemic toxicity in this study was considered to be 120 mg/kg/dose and the NOAEL for local toxicity in this study was considered to be 20 mg/kg/dose. The corresponding AUC, and Cₘₐₓ of HSA-IGF2-R61A following the last dose (Day 16) at 20 mg/kg/dose were 267,000 h*ng/mL and 7720 ng/mL for males, and not calculatable and 11,000 ng/mL for females, respectively. In addition, it should be noted that exposure on Day 16 is notably lower than on Day 1, likely due to the presence of ADAs.

4-Week GLP Study in Rats with Recovery (Ongoing: GLP-Tox-Rat-01), GLP, FED

The study design is shown in Table 11; the objective of the repeat-dose GLP study in rats is to determine the potential toxicity of HSA-IGF2-R61A when administered SC every three days for 4-weeks (total of 10 doses) under FED conditions, followed by a 4-week recovery period to monitor reversibility. The FED conditions prevent the hypoglycemia observed in some pilot studies under fasted conditions. In addition, TK and PD markers are also being evaluated. The dose levels for this study were based on the previous SC DRF study above. The high dose level of 60 mg/kg/dose was selected due to the poor local tolerability of the 120 mg/kg/dose level in the DRF study.

TABLE 11
Sprague Dawley Rat GLP Study Design (Study: GLP-Tox-Rat-01)

| | | | Dose | Main Study | Recovery | Toxicokinetic |
|---|---|---|---|---|---|---|
| | | Dose | Volume | Animals | Animals | Animals |
| Group | Treatment | (mg/kg/dose)^{a} | (mL/kg) | (M/F) | (M/F) | (M/F) |
| 1 | PBS | 0 | 1.5 | 10/10 | 5/5 | 6/6 |
| 2 | HSA-IGF2- | 3 | 0.075 | 10/10 | 5/5 | 6/6 |
| | R61A | | | | | |
| 3 | HSA-IGF2- | 10 | 0.3 | 10/10 | 5/5 | 6/6 |
| | R61A | | | | | |
| 4 | HSA-IGF2- | 60 | 1.5 | 10/10 | 5/5 | 6/6 |
| | R61A | | | | | |

Abbreviations: F: female; M: male; PBS: Phosphate buffered saline
^{a}HSA-IGF2-R61A was administered at a fixed concentration of 40 mg/mL.

Test article related effects on standard toxicological endpoints including clinical observations, post-dose observations, injection site observations, body weights, food consumption, ophthalmic examinations, evaluation of skin reactions, hematology, clinical chemistry, coagulation, urinalysis, organ weights, macroscopic and microscopic histopathology were evaluated. In addition to the standard parameters, effects on blood glucose levels, a PD marker, were monitored using a glucometer at multiple timepoints on each dosing occasion. Samples for TK were collected for evaluation throughout the study.

A single mortality was observed on Day 22. Animal 4512 (female, 60 mg/kg) was found dead on Day 22 prior to dosing. A blood sample was taken from this animal earlier in the day and at that time this animal was normal. No HSA-IGF2-R61A-related macroscopic or microscopic findings were observed and this death was considered procedural and not HSA-IGF2-R61A-related. Otherwise, results indicate that HSA-IGF2-R61A was systemically well tolerated during the 4-week dosing period at all doses.

HSA-IGF2-R61A-related effects were limited to the injection sites and the draining lymph nodes associated with them. No systemic effects were observed, possibly due to the lack of systemic exposure after the first few days of the study.

HSA-IGF2-R61A-related effects on Day 29 were characterized macroscopically by gelatinous swelling in the SC sites of two females at 3 mg/kg/dose and enlargement of draining lymph nodes in one male at 60 mg/kg/dose and six females at ≥3 mg/kg/dose, with microscopic increased mixed cell infiltrates, hemorrhage, pseudocyst formation, and/or edema in SC injection sites in males and females at ≥3 mg/kg/dose, and in draining lymph nodes by increased incidence of lymphoid cellularity in one male at 10 mg/kg/dose, one male at 60 mg/kg/dose, and in females at ≥10 mg/kg/dose. On Recovery Day 57, microscopic findings noted at the terminal euthanasia were almost completely resolved in SC sites, except for minimal hemorrhage in one female (Animal No. 2514) at 3 mg/kg/dose. Persistent changes in axillary lymph nodes consisted of enlargement in one female at 60 mg/kg/dose with correlative mild increased lymphocytes; additional increased lymphocytes in draining lymph nodes, without a macroscopic change, were also noted in one male each at 3 and 60 mg/kg/dose and in two females at 3 mg/kg/dose and one female at 60 mg/kg/dose. Changes in the SC sites were considered almost completely resolved, while only partially resolved in the draining lymph nodes.

The lymphatic route is the main venue for absorption of biologics following SC dosing. Therefore, these findings at the SC sites and the draining lymph nodes are consistent with the administration of a fully human protein to a rat by the SC route. Additionally, the compartmentalization of cellular and molecular mechanisms involved in the generation of antigen-specific humoral responses may further contribute to the development of potential anti-drug antibody response to administered fully human protein drug in rats.

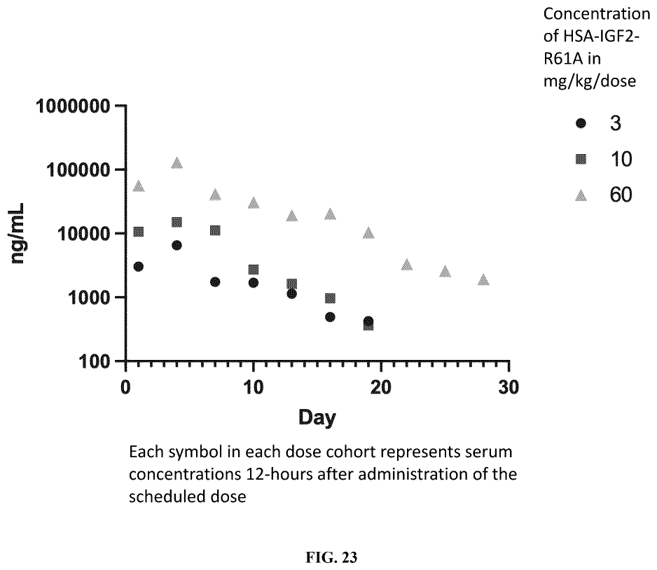

Bioanalytic and TK results are in progress. Limited bioanalytic data is available and has not been through a formal QC process yet. In order to closely monitor the drastic drop in exposure, a bioanalytic timepoint was collected at 12 hours post dose (approximately the Tₘₐₓ) after every dose. FIG. 23 shows that the accumulation occurred between the first and second doses (Day 1 and Day 4). Following the second dose, a drastic drop in exposure is observed, approaching near zero (below LLOQ) by Day 22 at the lower doses and barely detectable at the high dose of 60 mg/kg/dose. The mid-study AUCₗₐₛₜ (Day 16) was notably lower than the Day 1 AUCₗₐₛₜ at all doses and the end of study AUC last (Day 28) was only calculatable at the highest dose level of 60 mg/kg/dose. No notable HSA-IGF2-R61A-related toxicity other than transient injection site-related findings has been reported at any administered dose following SC administration of HSA-IGF2-R61A every 3 days for 1-month. Given the minimal exposure observed in longer duration studies, the Sponsor does not plan to use the rat as a toxicology species in studies lasting longer than a few days. In summary, the NOAEL was 60 mg/kg/dose under FED conditions when administered every 3 days for 1-month with a 1-month recovery.

Dose Range Finding study in Dogs (Ongoing: Dog_JUV_2023_DRF_01), Non-GLP, FED

The study design is shown in Table 12; the objective of the planned repeat-dose DRF study is to determine the potential toxicity of HSA-IGF2-R61A when administered SC every three days for approximately 2-weeks under FED conditions in order to enable dose selection for the GLP repeat dose toxicity study. The FED conditions prevent the hypoglycemia observed in some pilot studies under fasted conditions. In addition, TK and PD markers were also evaluated. The doses for this study were based on the IV MTD, PK and PD study adjusted for an estimated SC bioavailability of approximately 50%.

TABLE 12
Beagle Dog Dose Range Finding Study Design

| | | Dose | Dose | Main Study |
|---|---|---|---|---|
| | | (mg/kg/ | Volume | Animals |
| Group | Treatment | dose) ^{a} | (mL/kg) | (M/F) |
| 1 | PBS | 0 | 1.25→0.75b | 2/2 |
| 2 | HSA-IGF2-R61A | 3 | 0.075 | 2/2 |
| 3 | HSA-IGF2-R61A | 10 | 0.25 | 2/2 |
| 4 | HSA-IGF2-R61A | 20 | 0.5 | 2/2 |
| 5 | HSA-IGF2-R61A | 50→30^{b} | 1.25→0.75b | 2/2 |

Abbreviations:
F: female;
M: male;
PBS: Phosphate buffered saline
^{a} HSA-IGF2-R61A was administered at a fixed concentration of 40 mg/mL.
^{b}After Animal 5502 died on Day 6, the other animals in Group 5 were placed on dose holiday until Day 13 (i.e. doses were administered to this group on Days 1, 4, 13, 16). When dosing resumed on Day 13, the dose administered was reduced to 30 mg/kg/dose.

Test-article related effects on standard toxicological endpoints including clinical observations, post-dose observations, injection site observations, body weights, food consumption, hematology, clinical chemistry, coagulation, urinalysis, organ weights, macroscopic and limited microscopic histopathology were evaluated. In addition to the standard parameters, effects on blood glucose levels, a PD marker, were monitored using a ONETOUCH Ultra Vue Glucometer at multiple timepoints on each dosing occasion. Samples for TK are being collected for evaluation throughout the study.

One female at 50 mg/kg/dose was found dead on Day 6 after the second dose was administered on Day 4. Test-article-related in-life observations included fluid under skin (dorsal surface, noted only on dose days), prostration, bilateral eyes, ptosis, and material about mouth (red/brown) after first dose. Prior to being found dead, the animal had zero food consumption on Day 5. Test-article-related lower blood glucose levels at 12 h post-dose, compared with 0 h, were noted up to −39.5% on Day 1, which showed recovery at 24 h post-dose, and were noted up to −62.5% on Day 4. Similar trends in blood glucose were observed in other animals at 50 mg/kg/dose (below). When compared with pretest, clinical pathology evaluations performed on Day 1 revealed slightly decreased platelets, increased neutrophils, and increased prothrombin time. At necropsy, test-article-related macroscopic observations included discoloration in multiple organs, which correlated microscopically with multi-organ hemorrhage. In addition, diffuse edema in the alveolus in the lungs with mainstem bronchi; increased cellularity in the white pulp of spleen; decreased secretion in the acinar cells of pancreas; decreased vacuolation in the zona fasciculate of the adrenal glands; and decreased cellularity of the lymphocytes in the cortex and medulla of thymus was also observed. The cause of death was considered to be test-article-related hemorrhage in multiple organs and markedly diffuse edema in the lungs.

Test-article-related clinical signs, including fluid under skin (dorsal surface), were noted in animals at ≥10 mg/kg/dose on dosing days and had fully resolved prior to the next day.

No test-article-related changes were observed on body weight/body-weight gain. Test-article-related lower food consumption was noted up to −68.3% in one female (4502) at 20 mg/kg/dose from Day 16 to Day 18, compared with controls.

Test-article-related decreases in blood glucose were noted up to −47.1%, −54.7%, and −62.5% in one male and two females at 12 h following the dose of 50 mg/kg/dose on Day 1 and/or Day 4 when compared with pre-dose levels.

No test-article-related changes were observed in hematology, or coagulation during the study. Test-article-related changes in serum chemistry and urinalysis were limited to 20 mg/kg/dose. Compared with pretest, test-article-related changes included increased urea (6-fold), creatinine (2-fold), high urine bilirubin (+++) and urine protein (+++) in one female (4502) at 20 mg/kg/dose. In addition, high urine specific gravity (6.3% compared with pretest) and urine protein (++++) were observed in one male (4002) at 20 mg/kg/dose at the end of the dosing period. These changes were considered adverse, as they correlated with observed hyperplasia of the mesangial cells and hyaline cast in the kidney.

Test-article-related organ weight changes consisted of increased kidney weights for both sexes at ≥20 mg/kg/dose, increased splenic weights for both sexes at ≥3 mg/kg/dose, decreased thymic weights for males at 3 mg/kg/dose and females at 20 mg/kg/dose, and decreased pancreas weights for both sexes at 3 mg/kg/dose. Test-article-related macroscopic changes consisted of multifocal dark red discoloration in the kidneys and enlarged kidneys in one male (4002) at 20 mg/kg/dose, diffuse pale discoloration in the kidneys, and a small thymus in one female (4502) at 20 mg/kg/dose.

Test-article-related microscopic changes consisted of multifocal/diffuse hyperplasia of the mesangial cells in the glomerulus of the kidneys in males at ≥20 mg/kg/dose and one female at 20 mg/kg/dose; multifocal hyaline cast in the tubules of the kidneys in both sexes at ≥20 mg/kg/dose; multifocal tubular dilatation in the kidneys; multifocal infiltration of mononuclear cells in the interstitium of the kidneys; scattered increased mitotic figure in the tubules of the kidneys; multifocal congestion in the kidneys in one male at 20 mg/kg/dose; increased cellularity in the white pulp of the spleen in both sexes at ≥3 mg/kg/dose; decreased lymphocytic cellularity in the cortex and/or medulla of the thymus in males at ≥10 mg/kg/dose and one female at 20 mg/kg/dose; increased single-cell necrosis of the acinar cells of the pancreas in males at ≥3 mg/kg/dose; multifocal/diffuse decreased secretion in the acinar cells in males at 3, 20, and 50→30 mg/kg/dose and females at ≥3 mg/kg/dose; and multifocal infiltration of mononuclear cells in the subcutis and/or muscularis in both sexes at ≥3 mg/kg/dose. These changes (hyperplasia of the mesangial cells and hyaline cast) in one male (4002) and one female (4502) at 20 mg/kg/dose correlated with the increased urine protein and specific gravity, and increased serum chemistry parameters of urea and creatinine, which indicates an effect on the renal function and were considered adverse at ≥20 mg/kg/dose. Other changes were considered non-adverse due to the low grade and severity.

After once-every-three days SC injection of HSA-IGF2-R61A at 3, 10, 20, or 50-30 mg/kg/dose to male and female dogs for up to 18 days, the toxicokinetic profiles (Table 13) of HSA-IGF2-R61A on Days 1, 10, and 16, summarized as mean Tₘₐₓ values, were observed between 18.0 and 48.0 hours post-dose; no marked sex difference in systemic exposure was observed at any dose level; the systemic exposure increased dose proportionally in both sexes on Day 1 as the dosage increased from 3 to 50 mg/kg/dose; and no marked drug accumulation was observed at 3, 10, and 20 mg/kg/dose (Day 10 over Day 1 and Day 16 over Day 1).

TABLE 13
Selected TK Parameters in Beagle Dogs Following Repeat Dose HSA-IGF2-
R61A SC Administration to Dogs (Study: Dog_JUV_2023_DRF_01), Non-GLP, FED

| Dose | Study | | Cₘₐₓ | Tₘₐₓ | AUCT | AUCτ/D |
|---|---|---|---|---|---|---|
| (mg/kg/dose) | Day | Sex | (ng/mL) | (h) | (h * ng/mL) | (h * kg * ng/mL/mg) |
| 3 | 1 | Male | 8300 | 36.0 | 449,000 | 150,000 |
| | | Female | 8630 | 36.0 | 441,000 | 147,000 |
| | 10 | Male | 12,400 | 18.0 | 641,000 | 214,000 |
| | | Female | 11,600 | 24.0 | 630,000 | 210,000 |
| | 16 | Male | 12,000 | 18.0 | 587,000 | 196,000 |
| | | Female | 10,400 | 12.0 | 528,000 | 176,000 |
| 10 | 1 | Male | 32,500 | 36.0 | 1,670,000 | 167,000 |
| | | Female | 34,700 | 36.0 | 1,700,000 | 170,000 |
| | 10 | Male | 43,400 | 24.0 | 2,340,000 | 234,000 |
| | | Female | 44,400 | 24.0 | 2,290,000 | 229,000 |
| | 16 | Male | 38,500 | 24.0 | 2,040,000 | 204,000 |
| | | Female | 29,100 | 24.0 | 1,290,000 | 129,000 |
| 20 | 1 | Male | 91,900 | 24.0 | 4,080,000 | 204,000 |
| | | Female | 72,100 | 36.0 | 3,690,000 | 185,000 |
| | 10 | Male | 104,000 | 24.0 | 5,140,000 | 257,000 |
| | | Female | 100,000 | 24.0 | 5,150,000 | 257,000 |
| | 16 | Male | 58,800 | 18.0 | 2,610,000 | 130,000 |
| | | Female | 56,000 | 18.0 | 3,140,000 | 157,000 |
| 50 ^{a} | 1 | Male | 180,000 | 48.0 | 9,490,000 | 1900,00 |
| | | Female | 195,000 | 36.0 | 10,400,000 | 208,000 |
| 30 ^{a} | 16 | Male | 90,800 | 24.0 | 4,800,000 | 160,000 |
| | | Female | 107,000 | 24.0 | 4,750,000 | 158,000 |

^{a} After Animal 5502 died on Day 6, the other animals in the 50 mg/kg group were placed on dose holiday until Day 13 (i.e. doses were administered to this group on Days 1, 4, 13, 16). When dosing resumed on Day 13, the dose administered was reduced to 30 mg/kg/dose.

Results: Administration of HSA-IGF2-R61A to dogs once every three days up to 18 days by SC injection at dosages of 3, 10, 20, or 50→30 mg/kg/dose resulted in early death of one female at 50 mg/kg/dose, and adverse effects in food consumption, serum chemistry, urinalysis, and pathology at ≥20 mg/kg/dose. Therefore, the no-observed-adverse-effect level (NOAEL) for this study was considered to be 10 mg/kg/dose. The corresponding AUCτ and Cₘₐₓ of HSA-IGF2-R61A following the last dose at the NOAEL were 2,040,000 h*ng/mL and 38,500 ng/mL for males, and 1,290,000 h*ng/mL and 29,100 ng/mL for females, respectively.

4-Week GLP Study in Beagle Dogs with Recovery (Ongoing: GLP-Tox-Dog-01), GLP, FED

The study design is shown Table 14; the objective of the planned repeat-dose GLP study is to determine the potential toxicity of HSA-IGF2-R61A when administered SC every three days for 1-month under FED conditions, followed by a 4-week recovery period to monitor reversibility. In addition, TK and PD markers are also being evaluated. The doses for this study were based on the previous SC DRF study: (Dose Range Finding study in Dogs (Ongoing: Dog_JUV_2023_DRF_01), Non-GLP, FED).

TABLE 14
Beagle Dog GLP Study Design

| | | Dose | Dose | Main Study | Recovery |
|---|---|---|---|---|---|
| | | (mg/kg/ | Volume | Animals | Animals |
| Group | Treatment | dose) ^{a} | (mL/kg) | (M/F) | (M/F) |
| 1 | PBS | 0 | 0.5 | 4/4 | 2/2 |
| 2 | HSA-IGF2-R61A | 0.5 | 0.0125 | 4/4 | 2/2 |
| 3 | HSA-IGF2-R61A | 2 | 0.05 | 4/4 | 2/2 |
| 4 | HSA-IGF2-R61A | 20 | 0.5 | 4/4 | 2/2 |

Abbreviations:
F: female;
M: male;
PBS: Phosphate buffered saline
^{a} HSA-IGF2-R61A was administered at a fixed concentration of 40 mg/mL.

Test-article related effects on standard toxicological endpoints including clinical observations, post-dose observations, injection site observations, body weights, food consumption, ophthalmic examinations, hematology, clinical chemistry, coagulation, urinalysis, organ weights, macroscopic and microscopic histopathology are being evaluated. Neurological, respiratory and cardiovascular safety pharmacology parameters are being monitored by neurological examination, manual count and ECG, respectively. In addition to the standard parameters, effects on blood glucose levels, a PD marker, were monitored using a glucometer at multiple timepoints on each dosing occasion. Samples for TK were collected for evaluation throughout the study.

A single mortality was observed on Day 28. Animal 4003 (male, 20 mg/kg) exhibited adverse clinical signs including labored breathing, decreased activity, and severely decreased spontaneous movements approximately 0.5 hours following dose administration (prior to dose administration no notable clinical signs were observed). The veterinarian diagnosed a hypersensitivity reaction and administered diphenhydramine. The diphenhydramine was initially effective, however 4003 continued to deteriorate and was euthanized later that day (approximately 3 hours post dose) for animal welfare reasons including vomiting blood. The underlying cause for morbidity was acute multisystemic hemorrhage, principally gastrointestinal (notably stomach and rectal) and considered HSA-IGF2-R61A related, which correlated with minimally decreased platelets (0.57×) as well as markedly increased activated partial thromboplastin time (APTT, up to 15.88×). Further evaluation is ongoing. Otherwise, initial results indicate that HSA-IGF2-R61A was systemically well tolerated during the 4-week dosing period at all doses.

In addition to the mortality described above, HSA-IGF2-R61A-related clinical observations included lameness and swelling in 1 female at 2 mg/kg and lameness, swelling/edema, and erythema in 3 females at 20 mg/kg. These incidences typically occurred following dosing and were considered likely secondary to hypersensitivity reaction. These females were administered Benadryl for most of the second half of the study, and these findings largely resolved and were no longer present following the cessation of dosing. A small mean body weight reduction was also observed in females at 20 mg/kg, which persisted through the recovery period.

Only very slight injection site findings were observed visually. However, microscopically, transient injection site changes were present in both males and females, in all dose groups, and the severity of findings was generally dose dependent. The transient injection site changes were characterized by one of the following: minimal to moderate acute hemorrhage, minimal to mild mononuclear cell infiltration, or mild to moderate mixed cell inflammation.

Excluding the mortality, no HSA-IGF2-R61A changes were observed in safety pharmacology parameters, ophthalmology, coagulation, clinical chemistry, urinalysis, or blood glucose levels.

A minimal transient decrease in red blood cells (RBC) mass and platelets was observed at in males at 20 mg/kg. At 20 mg/kg/day, HSA-IGF2-R61A administration resulted in minimal to mild gastrointestinal hemorrhage in one animal and 1 of 3 males and 3 of 4 females demonstrated a kidney impact. Kidney changes were characterized by minimal to mild mononuclear inflammatory cell infiltration. Following a 28-day recovery period (Day 56) there were no HSA-IGF2-R61A-related organ weight changes, macroscopic, or microscopic findings at doses up to 20 mg/kg/day. HSA-IGF2-R61A associated injection site, gastrointestinal, and renal effects were not observed on Day 56, indicating resolution.

In order to monitor the exposure, a bioanalytic timepoint was collected at 12 hours post dose (approximately the Cₘₐₓ) after every dose. shows that similar Cmax exposure was observed throughout the entire study at ≥2.0 mg/kg/dose. Exposure was also maintained in individual animals at 0.5 mg/kg/dose.

Exposure is expected to be similar to that in the DRF study above. Toxicity observed at 20 mg/kg/dose included a single mortality associated with acute GI hemorrhage, transient minimal to mild findings in the kidney, transient injection site findings and hypersensitivity. The dog NOAEL under FED conditions when administered every 3 days for 1-month was 2.0 mg/kg/dose. Exposure was maintained throughout the study at the 2.0 mg/kg/dose NOAEL.

### General Toxicology Study

3-month multiple dose toxicology studies with recovery is undertaken in the dog to support a Phase 2 Adult DM1 patient study (see Table 13). 3-month studies are not planned in the rat due to the drastic decline in exposure over time (FIG. 22A-FIG. 22B). Longer term general toxicities studies are undertaken and the design of those studies is based upon the results of the 3-month dog study. Chronic toxicity, carcinogenicity, reproductivity toxicity and developmental toxicity risks are evaluated.

### 3-Month Repeat Dose Dog GLP Study

The objective of the 3-month repeat-dose GLP study is to determine the potential toxicity of HSA-IGF2-R61A when administered SC every three days for approximately 3-months. This study may also evaluate the potential reversibility of any findings following an approximately 1-month recovery period. In addition, safety pharmacology parameters, TK and exploratory PD markers are evaluated. Doses were selected based on the 1-month GLP study. The study design is shown in Table 15.

TABLE 15
3-Month Beagle Dog GLP Study Design

| | Dose | Main Study | | Recovery (if included) | |
|---|---|---|---|---|---|
| Group | mg/kg | Male | Female | Male | Female |
| 1 | PBS Control | 4 | 4 | 2 | 2 |
| 2 | low | 4 | 4 | 2 | 2 |
| 3 | mid | 4 | 4 | 2 | 2 |
| 4 | high | 4 | 4 | 2 | 2 |
| | Total: | 16 | 16 | 8 | 8 |

| | |
|---|---|
| Animal Species: | Beagle Dogs |
| Route of | Subcutaneous; alternating sites |
| Administration: | |
| Dose Frequency: | Every 3^{rd} day |
| Termination: | Day 91 (main study), Day 119 (recovery; if |
| | included) |

Safety Endpoints

| | |
|---|---|
| Cageside | Once daily with twice daily mortality/moribundity; |
| Observations: | beginning Week −2 |
| Detailed Clinical | At least once during pretest for all animals, once |
| Observations: | weekly thereafter |
| Injection Site | weekly |
| Observations: | |
| Food Consumption: | Twice daily |
| Body Weights: | Standard |
| Ophthalmology: | Standard |

Safety Pharmacology Endpoints

| | |
|---|---|
| Cardiovascular JET: | Pretest one day during Weeks 1 and 12. Recovery |
| | animals are examined again prior to recovery |
| | necropsy if treatment related effects are found |
| | during Week 12 exams |
| Neurological FOB: | Pretest and at 24 hrs (+/−4 hrs) post dose |
| | one day during Weeks 1 and 12. Recovery animals |
| | are examined again prior to recovery necropsy if |
| | treatment related effects are found during Week |
| | 12 exams |
| Visual Assessment | Pretest and at 24 hrs (+/−4 hrs) post dose |
| of Respiratory | one day during Weeks 1 and 12. Recovery animals |
| Rate | are examined again prior to recovery necropsy if |
| | treatment related effects are found during Week |
| | 12 exams |

Clinical Pathology

| | |
|---|---|
| Hematology, Clin | Pre-study: Twice |
| Chem, Coagulation, | 7 occasions |
| Urinalysis: | Prior to termination |
| Blood Glucose | Blood glucose is evaluated via glucometer at the |
| | same time points as TK sample collection below. |

Pathology

| | |
|---|---|
| Necropsy: | All study animals |
| Organ weights: | Standard organ weight |
| Histopathology: | standard list including both injection sites; |
| | all animals |
| ADA | At least Prestudy, monthly |

Bioanalytical
Plasma Sample Collection Time Points

| | | Time Points |
|---|---|---|
| Animals | Study Day or Week | (Relative to Dosing) |
| All | At least First dose, Day 29, | Full TK: 0 (predose), 1, |
| | Day 85, Last dose | 6, 12, 24, and 48 h postdose |
| All | other dosing days (optional - | Peak/Trough |
| | TBD after TK data from 1- | |
| | month study is evaluated) | |
| Recovery | Weekly during recovery | Single time point |
| Animals | | |

Genotoxicity and Carcinogenicity: HSA-IGF2-R61A is a recombinant protein made up entirely of naturally occurring amino acids and contains no inorganic or synthetic organic linkers or other non-protein portions. This indicates that it is highly unlikely that HSA-IGF2-R61A would react directly with DNA or other chromosomal material. Therefore, no genotoxicity assays are planned, in accordance with ICH S6. Standard carcinogenicity bioassays are generally inappropriate for biotechnology-derived pharmaceuticals such as HSA-IGF2-R61A. Therefore, the carcinogenic risks of HSA-IGF2-R61A are evaluated as described for protein therapeutics in ICH guidelines.

Local Tolerance: No stand-alone local toxicity studies are currently planned; however, consistent with ICH S6, evaluation of local tolerance (injection site clinical observations, macro- and microscopic examination of tissue samples from the injection site) are included as part of the repeat-dose toxicology studies. Reversible local tolerability findings were observed in both species.

Local tolerability was the dose-limiting toxicity in the rat. Macroscopically gelatinous swelling at the injection site and enlarged draining lymph nodes were observed. These findings were accompanied by microscopic observations of increased mixed cell infiltrates, hemorrhage, pseudocyst formation, and/or edema. These local findings were reversible.

In the dog, only very slight injection site findings were observed visually. However, microscopically, injection site changes were observed including minimal to moderate acute hemorrhage, minimal to mild mononuclear cell infiltration, and/or mild to moderate mixed cell inflammation. These local findings were reversible.

### Nonclinical Immunogenicity Assessment:

As human protein therapeutics can induce immune responses in animal species, the development of the relevant ADA can limit the evaluation of the study outcomes with effect on PK (anti-isotypic and anti-idiotypic antibodies) and PD (anti-idiotypic antibodies) in various animal species. To ensure appropriate interpretation of the nonclinical safety data in the absence of a potential ADA response, the standard bridging ELISA methods are developed where HSA-IGF2-R61A is used as both the capture and detection. Appropriate positive controls have been generated or acquired to determine the method sensitivity, drug tolerance, and conditions for false positive or false negative. This method is validated according to the relevant regulatory guidance(s) and support the IND-enabling GLP studies. Additionally, similar validated assays are used in support of the clinical development of HSA-IGF2-R61A. It is also the applicant's intention to establish a NAB in support of the proposed clinical studies. The configuration and details of the NAB assay are submitted with the IND. Additionally, as the approach to the assessment of immunogenicity, in vivo is a composite approach, the results from various ADA assays are evaluated in combination with the animal or patient HSA-IGF2-R61A exposure-time profile (PK) and the relevant safety or activity markers.

### Example 9—Phase 1 Combined Single-Ascending Dose (SAD) and Multiple-Ascending Dose (MAD) Clinical Study in Humans for HSA-IGF2-R61A

The focus of the study in this Example is assessment of safety and tolerability, PK, and PD in healthy volunteers. Based on data from the study, doses and dosing regimens are selected to take into future studies in patients with adult-onset DM1. Table 16 provides Phase 1 study synopsis.

TABLE 16
Synopsis of First in Human (FIH) Healthy Volunteer SAD/MAD Study

| | |
|---|---|
| Objective | Endpoint |

Primary

| | |
|---|---|
| To evaluate the safety, tolerability | Changes from baseline following single- and multiple-dose |
| and immunogenicity of HSA-IGF2- | subcutaneous administration of study drug (HSA-IGF2-R61A or |
| R61A administered subcutaneously | placebo): |
| in single ascending doses (SAD) | Treatment-emergent adverse events |
| and multiple ascending doses | Vital signs, physical examination |
| (MAD) to healthy adults | Clinical laboratory tests (hematology, chemistry, coagulation, |
| | urinalysis) |
| | ECG parameters (rhythm, heart rate [HR], PR, QRS, and |
| | QTcF intervals) |
| | Presence (titers) of anti-drug antibodies (ADA) and anti- |
| | HSA-IGF2-R61A neutralizing antibodies (Nab) |

Secondary

| | |
|---|---|
| To characterize the single- and | Pharmacokinetic Profile (including, but not limited to): |
| multiple-dose plasma | Time to maximum concentration (Tₘₐₓ) |
| pharmacokinetic (PK) profile of | Maximum observed concentration (Cₘₐₓ) |
| HSA-IGF2-R61A | Area under the curve from time zero to infinity (AUC_{(0-∞)}) |
| | Area under the curve from time zero to last measurable |
| | concentration (AUC(₀₋ₗₐₛₜ₎) |
| | Terminal half-life (t_{1/2}) |

Exploratory

| | |
|---|---|
| To preliminarily evaluate | Changes from baseline following single- and multiple-dose |
| pharmacodynamic responses to | subcutaneous administration of study drug (HSA-IGF2-R61A or |
| administration of HSA-IGF2-R61A | placebo): |
| | Oral glucose tolerance test |
| | Biomarkers related to target engagement |
| | Biomarkers related to metabolism |
| | Biomarkers related to myogenesis |

### Study Rationale

HSA-IGF2-R61A is being developed to treat symptoms associated with adult-onset, myotonic dystrophy type 1 (DM1). HSA-IGF2-R61A has not been previously administered in human subjects.

The present First-In-Human (FIH) study (HSA-IGF2-R61A-101) aims to assess the safety, tolerability, and pharmacokinetics (PK) of single and repeated subcutaneous doses of HSA-IGF2-R61A in healthy volunteers. The study design is well-established for FIH studies and appropriate to assess the preliminary safety and tolerability of new drug candidates.

This study is also designed to support future clinical development of HSA-IGF2-R61A in other degenerative myopathies and other disorders for which preclinical efficacy data have been obtained.

### Trial Design

This is a first-in-human, randomized, double-blind (Sponsor unblinded), placebo-controlled, combined single-ascending dose (SAD) and multiple-ascending dose (MAD) study. The study is conducted at a single center and includes healthy volunteer subjects.

### Single-Ascending Dose (SAD)

The SAD part consists of 5 cohorts of 8 healthy adult volunteer subjects each of whom receive a single dose of study drug (HSA-IGF2-R61A, n=6; placebo, n=2).

For the SAD cohorts, after consideration of all relevant information obtained from in vitro and nonclinical toxicity studies was used to estimate no observed adverse effect levels (NOAELs) and a human equivalent dose (HED). Based on this information, the planned starting dose of 0.01 mg/kg administered SC under fed conditions was selected to mitigate unknown or theoretical risks associated with HSA-IGF2-R61A. Sentinel dosing is implemented within each cohort as an additional safety measure to enable detection of acute safety risk(s). The recommended FIH dose should provide an approximately 300-fold lower exposure in humans relative to the NOAEL in dogs.

### Cohorts:

Cohort S1: A single dose of HSA-IGF2-R61A Dose Level S1 (0.01 mg/kg) (n=6) or matching placebo (n=2) on D1
Cohort S2: A single dose of HSA-IGF2-R61A Dose Level S2 (0.1 mg/kg) (n=6) or matching placebo (n=2) on D1.
Cohort S3: A single dose of HSA-IGF2-R61A Dose Level S3 (1.0 mg/kg) (n=6) or matching placebo (n=2) on D1.
Cohort S4: A single dose of HSA-IGF2-R61A Dose Level S4 (2.0 mg/kg) (n=6) or matching placebo (n=2) on D1.
Cohort S5: A single dose of HSA-IGF2-R61A Dose Level S5 (4.0 mg/kg) (n=6) or matching placebo (n=2) on D1
   The initial starting dose is based on all relevant information from non-clinical safety testing studies from which a human equivalent dose and expected no observed adverse event level are calculated. Subsequent doses are designated as multiples of the first dose level. D=day

In each cohort, 2 subjects (1 HSA-IGF2-R61A and 1 placebo) receive Study Drug on Study Day 1 (sentinel dosing). In Cohort 1, following a 72-hour period to detect the occurrence of reactions or significant adverse events (AEs) if the safety and tolerability results are acceptable, 3 subjects are dosed on Study Day 4. Assuming no significant safety issues are identified, this interval is shortened to 48 hours for subjects participating in Cohorts 2 through 5. For all 5 Cohorts, following an additional 48-hour period, if safety and tolerability results are acceptable, the final 3 subjects in the respective Cohorts receive Study Drug.

A Safety Review Committee (SRC) conducts two Safety Reviews per dosing Cohort.

On Study Day 3, the SRC reviews available clinical (including adverse events) and laboratory data to assess initial safety and tolerability of study drug. Assuming no safety issues are identified, dosing proceeds as described above.

On Study Day 28 (Day 21 for Subjects 6, 7 and 8), the SRC reviews available pharmacokinetic (PK) data, laboratory data and clinical observations, including adverse events, to assess safety and tolerability. Assuming no significant safety issues are identified, previously-screened subjects may proceed to the next assigned dose in the next Cohort. Alternatively, the SRC may determine that dose level adjustments be made or that Study Drug dosing be discontinued.

A sixth study cohort may be added, pending approval by the SRC, if the Sponsor believes additional dosing, safety, or pharmacokinetic data are required.

Study-related activities for subjects participating in the SAD portion of the study are described below. See Schedule of Activities (Table 18).

Conservative estimates for bioavailability (100% following SC dosing) and fast absorption rate (similar to that observed in dogs and faster than that reported for albumin fusion proteins) are likely to yield an overestimation of the anticipated exposure profiles in FIH PK projections following SC administration. Data from pre-clinical PK studies with HSA-IGF2-R61A in both dogs and rats indicate linear properties across a wide dose range in both species following IV and SC dosing. PK properties are similarly assumed to be linear following human exposure.

Dog was selected as the most pharmacologically sensitive and relevant species for evaluation of the HSA-IGF2-R61A PK, PD, and in GLP toxicology studies. Plasma concentration-time data for HSA-IGF2-R61A in dogs were described by a two-compartment PK model following IV and SC dosing. Plasma clearance for HSA-IGF2-R61A was assumed to be similar to that for Alb-IFN; other PK parameters are assumed to be similar to those from a high-resolution canine PK study.

Based on these studies and observations, the predicted Tₘₐₓ following SC administration of each single dose of HSA-IGF2-R61A is approximately 29 hours and the predicted half-life of HSA-IGF2-R61A is approximately 140 hours (i.e., approximately 5.9 days).

### Multiple-Ascending Dose (MAD)

The MAD part consists of 4 cohorts of 8 healthy adult volunteer subjects each of whom receives study drug (HSA-IGF2-R61A, n=4; placebo, n=2) weekly×3 (Study Days 1, 8, 15).

For the MAD cohorts, the dose range of 0.1 mg/kg to 4.0 mg/kg SC was chosen based on a predicted efficacious dose of 1 mg/kg SC once every week. Subjects were confined for a day prior to the first dose through at least 96 hours after the first dose. This design was chosen to optimize efficiency, while prioritizing safety and employing a conservative, stepwise approach, allowing SAD and MAD regimes to be conducted simultaneously. Progression to higher MAD doses (2.0 mg/kg and 4.0 mg/kg) are allowed after review of the safety and PK of cumulative SAD and MAD data available at that time.

### Cohorts:

Cohort M1: HSA-IGF2-R61A Dose Level M1 (0.1 mg/kg) (n=6) or matching placebo (n=2) SC QWX3
Cohort M2: HSA-IGF2-R61A Dose Level M2 (1.0 mg/kg) (n=6) or matching placebo (n=2) SC QWX3
Cohort M3: HSA-IGF2-R61A Dose Level M3 (2.0 mg/kg) (n=6) or matching placebo (n=2) SC QWX3
Cohort M4: HSA-IGF2-R61A Dose Level M4 (4.0 mg/kg) (n=6) or matching placebo (n=2) SC QWX3
   First dose level in Cohort M1 has safely administered to subjects participating in the SAD portion of the study prior to administration in the MAD portion. D=day

In each cohort, 2 subjects (1 HSA-IGF2-R61A and 1 placebo) receive Study Drug on Study Day 1 (sentinel dosing). In Cohort 1, following a 72-hour period to detect the occurrence of reactions or significant adverse events (AEs) and if the safety and tolerability results are acceptable, 3 subjects are dosed on Study Day 4. Assuming no significant safety issues are identified, this interval is shortened to 48 hours for subjects participating in Cohorts 2 through 4. For all 4 Cohorts, following an additional 48-hour period, if safety and tolerability results are acceptable, the final 3 subjects in the respective Cohorts receive Study Drug.

Assuming no safety issues have been identified, Cohort M1 receive the first dose of Study Drug (Study Day 1) on the day following successful review of safety data for SAD Cohorts 1 through 3.

An SRC conducts two Safety Reviews per dosing Cohort.

On Study Day 9, the SRC reviews available clinical (including adverse events) and laboratory data to assess initial safety and tolerability of study drug.

On Study Day 34 (Day 25 for Subjects 3-5 and Day 23 for Subjects 6-8), the SRC reviews available pharmacokinetic (PK) data, laboratory data and clinical observations, including adverse events, to assess safety and tolerability. Assuming no significant safety issues are identified, previously-screened subjects may proceed to the next assigned dose in the next Cohort. Alternatively, the SRC may determine that dose level adjustments be made or that Study Drug dosing be discontinued.

Study-related activities for subjects participating in the MAD portion of the study are described below. See Schedule of Activities (Table 19).

### Subject Population

Since the first target indication for HSA-IGF2-R61A is adult-onset myotonic dystrophy type 1 (DM1), this study includes subjects who are ≥18 years of age. Otherwise, for enrollment, there are no limitations with regard to gender, ethnicity or racial background.

SAD: The subject population is comprised of approximately 40 healthy normal volunteers (HNV), males or females, ages 18 through 60 inclusive.

MAD: The subject population is comprised of approximately 32 healthy normal volunteers (HNV), males or females, ages 18 through 60 inclusive.

### Dose & Dose Justification

The dose selection of HSA-IGF2-R61A is based on the No Observed Adverse Effect Level (NOAEL) in animals and the anticipated PK and pharmacodynamic (PD) responses in humans in combination with available non-clinical biology, toxicology and PK data. Calculation of the maximum recommended starting dose (MRSD) is based on the NOAEL converted into a human equivalent dose (HED) with an appropriate safety factor to derive the MRSD. This methodology is consistent with guidance from the United States Food and Drug Administration (FDA) and the European Medicines Agency (EMA) in order to identify and mitigate risks for first-in human clinical trials with investigational medicinal products.

The range of selected doses (from the MRSD to the maximal dose to be tested) is determined to ensure the safety of subjects participating in the study and to cover doses at which HSA-IGF2-R61A is expected to exert a beneficial effect in patients affected with DM1.

The SAD portion of the study is initiated first. Five dose levels of HSA-IGF2-R61A are incorporated into the SAD portion of the study. Following completion of Cohort 1, subjects participating in subsequent Cohorts receive study drug levels, based on adequate safety review, that are multiples of the initial dose level.

The MAD portion of the study begins after the completion of SAD Cohorts S1, S2 and S3. Assuming adequate safety and tolerability, the first MAD dose level administered in Cohort M1 is a dose level previously administered to subjects participating in the SAD portion of the study. Following completion of Cohort M1, subjects participating in subsequent Cohorts receive study drug levels, based on adequate safety review, that are multiples of the initial dose level. Dosing during the MAD portion of the study is weekly×3 (Study Days 1, 8 and 15).

### Route of Delivery

Doses are administered subcutaneously (SC).

### Eligibility Criteria (Applicable for Both SAD and MAD Portions of Study)

### Inclusion Criteria

Subjects must meet all the following criteria at Screening and on admission to the Study Unit (Day −1):
1. Are males or nonpregnant females, ages 18 to 60 (inclusive) at time of signing Informed Consent with body mass index (BMI) 18 to 35 kg/m².
2. Are willing and able to give informed consent and follow all study procedures and requirements.
3. Are able to understand the requirements of the study protocol.
4. Agree to complete all required study visits.
5. Are healthy based on physician examination, medical history laboratory tests, vital signs and resting electrocardiograms.
6. Are non-smokers or users of fewer tobacco- or nicotine-containing products equivalent to 5 cigarettes a day.
7. Are willing to abstain from caffeine and nicotine while in the Study Unit.
8. Have negative screens for alcohol and drugs of abuse at screening and admission.
9. Have no history of psychiatric disorders.
10. Are willing to abstain from all alcoholic beverages and cannabinoids for 48 h prior to dosing through the End-of-Study (EOS) visit.
11. Females must be either:
   of non-childbearing potential (defined as having undergone surgical sterilization (hysterectomy, bilateral salpingectomy, bilateral oophorectomy) or being postmenopausal (i.e., greater than 45 years old with amenorrhea for ≥12 months AND have a serum follicle stimulating hormone (FSH) level >40 mIU/mL)
OR
   Females must be of child-bearing potential and using at least one of the following acceptable methods of contraception from the time of informed consent through the time of study drug administration and for 8 weeks after last administration of study drug [calculation based on 5 HSA-IGF2-R61A half-lives+28 days; assume HSA-IGF2-R61A half-life of 6 days]:
      a. Stable use of hormonal contraceptive treatment (including oral, intravaginal or transdermal combined (estrogen- and progestogen-containing) contraception, oral or injectable progestogen-only contraception, and intrauterine hormone-releasing systems)
      b. Intrauterine device (IUD, including hormone-releasing IUDs)
      c. Monogamous relationship with a vasectomized partner
      d. Sexual abstinence (i.e., refraining from heterosexual intercourse) for 8 weeks prior to signing informed consent.
12. Men who are sexually active must use the following forms of medically acceptable birth control during the study drug treatment period and for 16 weeks after the last administration of study drug [calculation based on 5 HSA-IGF2-R61A half-lives+90 days; assume HSA-IGF2-R61A half-life of 6 days]:
   a. Vasectomy with medical assessment of surgical success
   OR
   b. Consistent use of a condom with partner also using either stable hormonal contraceptive or IUD.
   c. Sperm donation is prohibited during the study and for up to 4 weeks after the last administration of study drug.
13. Have NOT participated in a clinical study utilizing an investigational agent within 28 days or within 6 half-lives of the investigational drug (whichever is longer) prior to Screening

### Exclusion Criteria

Participants must meet NONE of the following at Screening and on admission (Day −1):
1. Are unwilling or unable to comply with study procedures, including follow-up, as specified by the protocol, or unwilling to cooperate fully with the Investigator.
2. Have a history of non-compliance with other therapies.
3. Have a history of drug or alcohol abuse within 3 months of Screening.
4. Have evidence of clinically significant abnormalities or disease, including, the following:
   a. Impaired renal function (estimated glomerular filtration rate [eGFR]<60 ml/min/1.73 m², using the CKD-EPI (CysC) equation)
   b. Urine dipstick results
      > trace protein; if present, a quantitative total urine protein measurement of <0.5 g/24 hours would enable study enrollment
      > trace blood; if present, urine microscopy showing <5 red blood cells per high power field would enable study enrollment
   c. Alanine aminotransferase (ALT) or aspartate aminotransferase (AST)>2.0× the upper limit of normal (ULN).
   d. Bilirubin >ULN (unless has a genetically confirmed diagnosis of Gilbert's syndrome)
   e. Platelet count <125×10⁹/L
   f Electrocardiogram (ECG) showing QTcF>470 msec female or >450 msec male
   g. Any other laboratory, vital sign, ECG abnormality, or clinical history or finding that, in the investigator's opinion, is likely to increase the risk of study participation, confound study results, or interfere with study conduct or adherence
   h. History of bleeding disorder or excessive bleeding
6. Have an active malignancy or have a history of malignancy within the 5 years prior to Screening. Subjects with prior basal or squamous cell carcinoma of the skin or carcinoma in situ of the cervix that has been successfully treated may be enrolled.
7. Have received treatment with
   a. another investigational drug, biologic agent, or device within 1 month of Screening, or 5 half-lives of investigational agent, whichever was longer.
   b. use of any prescription medication within 4 weeks (exception: contraceptives are permitted).
   c. use of any non-prescription medication within 5 days prior to dosing (exception: acetaminophen up to 2 g per day prior to dosing is permitted).
8. Have any other active medical conditions or any clinically significant laboratory abnormality, medical or psychiatric illness which, in the opinion of the Investigator, could interfere with the conduct or interpretation of the study or put the participant at risk
9. Have any of the following known active infections:
   a. Infection requiring systemic antiviral or antimicrobial therapy that would not have been completed prior to Study Day 1.
   b. HIV not optimally controlled or treated. Participants with HIV who are on sustained stable antiretrovirals (for >4 weeks) and have CD4+ counts ≥350 cells/μL may be enrolled. No HIV testing is required unless mandated by local health authority.
   c. Chronic hepatitis B virus (HBV) infection with surface antigen positive: subjects with a prior history of treated HBV infection who are hepatitis B surface antigen-negative may be enrolled. No testing is required for hepatitis B unless mandated by local health authority.
   d. Chronic hepatitis C virus (HCV) infection: untreated or on active treatment. Participants with a prior history of treated HCV infection who are HCV RNA-undetectable may be enrolled. No testing is required for hepatitis C unless mandated by local health authority.
10. Prior exposure to HSA-IGF2-R61A
11. History or immune reaction to any biologic therapy.
12. Donation or loss of greater than 1 unit (450 mL) of blood within 1 month prior to dosing

### Trial Enrollment Targets

This is a first-in-human study for HSA-IGF2-R61A and is conducted at a single study center. The study design incorporates integrated single-ascending dosing (SAD) and multiple-ascending dosing (MAD) portions. Dosing cohorts in both the SAD and MAD portions of the study includes 8 subjects. The overall sample size for this study, as described below, is consistent with first-in-human studies. The target disease affects adults; accordingly, no pediatric subjects are enrolled.

SAD: The goal for SAD portion of the study is to enroll approximately 40 normal healthy volunteer subjects (males and females).

MAD: The goal for this study is to enroll approximately 32 patients (males and females) normal healthy volunteer subjects (males and females).

### Treatment Duration

Total duration of participation, including screening, for an individual subject ranges from up to 55 days (SAD) to up to 71 days (MAD).

### SAD

Screening Period: Up to 35 days (Including Day −1).
Treatment Period: A single dose of Study drug (HSA-IGF2-R61A or placebo) is administered on Day 1. Subjects are observed for safety for 2 days in the SU.
Follow-up: 19 days after study drug administration. The subject has out-patient visits on Study Days 5, 7, 10, 15 and 20. Study personnel contact the subject on Study Days 13 and 17 to review status and adverse events.

### MAD

Screening Period: Up to 35 days (Including Day −1).
Treatment Period: 15 days. Study drug (HSA-IGF2-R61A or placebo) is administered weekly×3 (Study Days 1, 8 and 15). Subjects is observed for safety in the SU and during outpatient study visits (Study Days 4 and 11). Study personnel contact the subject on Study Days 3 and 10 to review status and adverse events.

Follow-up: 21 days. During the Follow-up Period, subjects return to the Study Site on Study Days 19, 22, 26, 29 and 36. In addition, subjects are contacted by study personnel to assess safety and tolerability on Study Days 18 and 33. The final study visit (End of Study) for a subject occurs on Study Day 36.

### Schedule of Evaluations

### SAD

See Schedule of Assessments for SAD Portion of Study (below).

### MAD

See Schedule of Assessments for MAD Portion of Study (below).

### Duration of Follow Up

### SAD

Following administration of study drug on Study Day 1, subjects are followed either in the study unit or as an outpatient until the final follow-up on Study Day 20.

### MAD

Following administration of study drug on Study Days 1, 8 and 15, subjects are followed either in the study unit or as an outpatient until the final follow-up on Study Day 36.

### Safety Considerations & Stopping Rules

All subjects are monitored closely by the CRU nursing staff and investigators during the procedures to prevent and minimize any potential risks or discomfort. The nursing staff and study personnel receive in-service training regarding these procedures. Each participant is asked to report any continuing discomfort caused by any of the performed procedures.

### Safety Monitoring Related to Mechanism of Action of HSA-IGF2-R61A

HSA-IGF2-R61A is a recombinant fusion protein (biologic) comprised of human serum albumen (HSA) linked to a human insulin-like growth factor-2 sequence (HSA-IGF2-R61A). Insulin-like Growth Factor-2 (IGF-2) is a mitogenic peptide hormone that is structurally similar to insulin. It binds to IGF-1R and the insulin receptor A (IR-A) on the surface of target cells. Two currently-marketed products incorporating IGF-1 (IPLEX® and INCRELEX®), used for treating growth hormone deficiency, may be associated with hypoglycemia. Doege-Potter syndrome, an extremely rare fibrous tumor that results in excess secretion of IFG-2, is likewise associated with hypoglycemia. Thus, hypoglycemia is a potential side effect of HSA-IGF2-R61A related to its mechanism of action. During the study, careful monitoring of glucose is performed, particularly in the 24 hours following administration of study drug. The study drug is administered in a FED state to mitigate post-dosing hypoglycemia. In addition, since subjects are confined in a SU for the first 7 days of the study, meals are administered at regular intervals. Additional snacks are also available.

Administration of products incorporating IGF-1 to children with growth hormone deficiency has been associated with minor elevations in hepatic enzymes. This study incorporates routine monitoring of hepatic enzymes through the duration of the dosing and follow-up periods.

### Immune Monitoring

Since human protein therapeutics may be associated with immune responses, subjects are monitored for anti-drug antibodies (ADAs) and neutralizing antibodies (NAbs) through validated activity assays during the course of the study. Sampling for immunogenicity occurs in parallel with pharmacokinetic sampling and at timepoints corresponding to predicted peak antibody responses.

### Stopping Rules for Individual Subjects

Subjects may voluntarily withdraw from the study for any reason at any time. Subjects are withdrawn from the study if intolerable side effects or severe adverse events, as determined by the PI, are identified. Subjects who cannot tolerate the medication or procedures, have surgery or other illnesses which affect the evaluations have the drug discontinued.

Specific stopping rules include:
ALT or AST values ≥3×ULN associated with total bilirubin ≥2×ULN, with no underlying medical conditions to explain the elevated values
Uncontrolled hypoglycemia

### Data & Safety Monitoring (DSM)

This study is conducted according to the ICH E6(R3) risk and quality processes described in the applicable procedural documents. Accurate eCRFs and source documentation are maintained. An external contract research organization (CRO) is responsible for activities associated with the data management of this study. This includes setting up a relevant database and data transfer mechanisms, along with appropriate validation of data and resolution of queries. Data generated within this clinical study are handled according to the relevant Standard Operating Procedures (SOPs) of CRO. Investigative site personnel enter subject data into an EDC system. The analysis data sets is a combination of these data and data from other sources (e.g., laboratory data). Clinical data management is performed in accordance with applicable CRO standards and data cleaning procedures to ensure the integrity of the data, e.g., removing errors and inconsistencies in the data. Adverse event terms are coded using MedDRA, an internal validated medical dictionary, and concomitant medications are coded using the World Health Organization Drug Dictionary.

A medical monitor reviews and assesses all safety data as they become available throughout the study.

A study monitor (clinical research associate) from the CRO monitors the study and site activities to verify that data are authentic, accurate, and complete; the safety and rights of subjects are being protected and the study is being conducted in accordance with the currently approved protocol and any other study agreements, good clinical practice (GCP) and all applicable regulatory requirements. The monitor checks that the CRFs have been correctly completed, with comparison of source documents and check that AEs have been documented. All relevant study-related documents and source data must be available at the study site to document the existence of the research subjects and to substantiate the integrity of study data collected. Source data must include the original documents relating to the study, as well as the medical treatment and medical history of the subject.

A Safety Review Committee (SRC) is comprised of at least three members, none of whom have direct participation in the conduct of the Study: two physicians (e.g., neurologist, internist) with experience in myotonic dystrophy and a statistician otherwise unaffiliated with the study. The SRC operates under a study-specific Charter that is agreed by the Committee members prior to screening the first study subject. At designated times during the study, the SRC reviews available pharmacokinetic (PK) data, laboratory data and clinical observations, including adverse events, to assess safety. Assuming no safety issues are identified, the SRC may make the recommendation, per protocol, that previously-screened subjects may proceed to the next assigned dose in the next scheduled Cohort. The SRC may also recommend that the next scheduled dose level be modified, dosing be paused or dosing be discontinued, or to modify the next dose or pause or discontinue the trial. Each SRC review and recommendation is documented in writing.

### Secondary Studies

PK assessments are conducted in this study. These data enable more precise dosing and follow-up during studies in patients with adult-onset myotonic dystrophy type 1.

PD assessments regarding glucose and insulin levels are obtained periodically during the study to monitor possible hypoglycemia. All study drug dosing occurs in the FED state to minimize the risk of hypoglycemia.

During the MAD portion of the study, clinical evaluations are correlated with study drug exposure. Other PD assessments (e.g., molecular biomarkers) may be performed during the SAD and/or MAD portions of the study.

### Assays & Methodologies

All assays being performed in this study are standardized (including blood levels of HSA-IGF2-R61A and anti-drug antibodies). No specialized methodology is needed to be developed or be employed during the study.

### Statistical Analysis

Three analysis populations are defined for this study.
The Safety Population is defined as all subjects who receive at least 1 dose of study drug and have at least 1 post-baseline safety assessment.
The PK Population is defined as all enrolled subjects for whom at least 1 PK parameter of interest can be calculated. In general, on a parameter-by-parameter basis, an individual subject's data may be excluded from analysis if insufficient data are available for that subject to calculate the specific parameter in question.
The Full Analysis Population is defined as all randomized subjects who complete the study without experiencing major protocol deviations or violations.

### Sample Size

The sample size is typical for first in human studies and allows a preliminary determination of tolerability safety and PD parameters; it also allows for a full, comprehensive determination of the single dose PK in healthy adults.

### Safety Analysis

The full detail of the safety analyses is outlined in a statistical analysis plan (SAP).

The safety analyses is assessed based on the evaluation of treatment-emergent AE (TEAE) for the entire study. All AEs are coded using the Medical Dictionary for Regulatory Activities (MedDRA) version 27.1 or later. Safety data, including AEs, clinical laboratory test results, 12-lead ECGs and Holter monitors, assessments of immunogenicity and vital sign measurements, are summarized by treatment. Physical examination findings and concomitant medications are listed.

### Pharmacokinetic Analysis

The full detail of the PK analyses is outlined in a statistical analysis plan (SAP). Briefly, the PK analyses is performed using the PK population. Plasma concentrations of HSA-IGF2-R61A are summarized by dose and time point using descriptive statistics. Mean and individual plasma drug concentrations over time are presented in figures using linear and semi log scales. The PK parameters listed above under “Endpoints” are calculated using a non-compartmental analysis and summarized by dose. AUC and Cmax are tested across dose levels for dose proportionality.

### Exploratory Pharmacodynamic Analyses

Change from baseline and absolute values for PD markers for the relevant pathways (TBD) is summarized by dose. Exploratory exposure response analyses for PD markers is performed starting with simple scatterplots (actual values and change from baseline) vs plasma drug concentration; further analyses including modeling of the response is performed as indicated.

### Long Term Follow Up

No long-term follow-up is envisioned for the healthy normal volunteers participating in this study. Table 17 shows study design dosing schema.

### Study Schedule for the SAD and MAD Cohorts

The SRC, at designated time points, reviews available clinical observations, reported adverse events, laboratory data and PK data to assess the safety and tolerability of the study drug (HSA-IGF2-R61A and placebo). In each cohort, 2 subjects (1=HSA-IGF2-R61A and 1=placebo) receives Study Drug on Study Day 1 (sentinel dosing). Assuming safety and tolerability are acceptable, the remaining six subjects are enrolled in each cohort sequentially in groups of three. These SRC reviews also trigger screening and study drug administration in subsequent cohorts.

Assuming no safety issues have been identified, Cohort MAD 1 receives the first dose of Study Drug (Study Day 1) on the day following successful review of safety data for SAD Cohorts 1 through 3. The arrow indicates when MAD dosing begins in relation to SAD dosing.

Table 17 represents the designated first dose level. This dose is determined based on all relevant information from non-clinical safety testing studies from which a human equivalent dose and expected no observed adverse event level are calculated. Subsequent doses are designated as multiples of the first dose level. Schedule of Activities (SoA)—SAD is listed in Table 18.

TABLE 18
Schedule of Activities (SoA)-SAD: Individual Subjects

| | Study Visit | | | | | | | | | | | | | | | | | | | | | | |
|---|---|---|---|---|---|---|---|---|---|---|---|---|---|---|---|---|---|---|---|---|---|---|---|
| | 1 | | 3 | 4 | 5 | | 6 | | | 7 | | 8 | | | 9 | | 10 | | 11 | | | 12 | |
| | Study Day | | | | | | | | | | | | | | | | | | | | | | |
| | −35 | −1 | 1 | 2 | 3 | 4 | 5 | 6 | 7 | 8 | 9 | 10 | 11 | 12 | 13 | 14 | 15 | 16 | 17 | 18 | 19 | 20 | |
| | Visit Location | | | | | | | | | | | | | | | | | | | | | | |
| Assessment | OP | SU | SU | SU | SU | OP | OV | OP | OP | OV | OP | OV | OP | OP | TC | OP | OV | OP | TC | OP | OP | OV | ET |
| Informed consent a | X | | | | | | | | | | | | | | | | | | | | | | |
| Demography | X | | | | | | | | | | | | | | | | | | | | | | |
| Medical/surgical | X | | | | | | | | | | | | | | | | | | | | | | |
| history | | | | | | | | | | | | | | | | | | | | | | | |
| Inclusion/exclusion | X | X | | | | | | | | | | | | | | | | | | | | | |
| criteria b | | | | | | | | | | | | | | | | | | | | | | | |
| CSSRS | X | X | | | | | | | | | | | | | | | | | | | | X | X |
| Height and weight c | X | X | | | | | | | | | | | | | | | | | | | | X | X |
| Physical | X | X | | | | | | | | | | | | | | | | | | | | X | X |
| examination d | | | | | | | | | | | | | | | | | | | | | | | |
| Vital signs e | X | X | X | X | X | | X | | | X | | X | | | | | X | | | X | | X | X |
| DOA screen f | X | X | | | | | | | | | | | | | | | | | | | | | |
| Pregnancy testing g | X | X | | | | | | | | | | X | | | | | X | | | X | | X | X |
| Serology (HIV, Hep | X | | | | | | | | | | | | | | | | | | | | | | |
| B/C) h | | | | | | | | | | | | | | | | | | | | | | | |
| Randomization i | | X | | | | | | | | | | | | | | | | | | | | | |
| HSA-IGF2-R61A or | | | X | | | | | | | | | | | | | | | | | | | | |
| placebo | | | | | | | | | | | | | | | | | | | | | | | |
| Adverse Events j | | X | X | X | X | | X | | | X | | X | | | X | | X | | X | X | X | X | X |
| Telephone Safety | | | | | | | | | | | | | | | X | | | | X | | X | | |
| Call k | | | | | | | | | | | | | | | | | | | | | | | |
| Laboratory Studies | X | X | X | X | X | | X | | | X | | X | | | | | X | | | X | | X | X |
| (see list) l | | | | | | | | | | | | | | | | | | | | | | | |
| Covid testing l | | | | | | | | | | | | | | | | | | | | | | | |
| Coagulation tests l | X | X | | | | | | | | | | X | | | | | X | | | X | | X | X |
| Urinalysis l | X | X | | | | | | | | | | X | | | | | X | | | X | | X | X |
| Glucose Tolerance | | X | | | | | | | | | | X | | | | | | | | | | | |
| Test l | | | | | | | | | | | | | | | | | | | | | | | |
| Insulin l | X | X | | | | | | | | | | X | | | | | X | | | X | | | |
| Hemoglobin A1C l | X | X | | | | | | | | | | X | | | | | X | | | X | | | |
| Lipid Profiles l | X | X | | | | | | | | | | X | | | | | X | | | X | | | |
| Insulin Sensitivity l | X | X | | | | | | | | | | X | | | | | X | | | X | | | |
| 12-lead ECG n | X | X | X | X | X | | X | | | X | | X | | | | | X | | | X | | X | X |
| Holter Monitor o | | | X | X | | | | | | | | | | | | | | | | | | | |
| Pharmacokinetics p | | | X | X | X | | X | | | X | | X | | | | | X | | | X | | X | X |
| Pharmacodynamics q | | | X | | X | | X | | | X | | | | | | | X | | | | | X | X |
| Immunogenicity r | | | X | | | | | | | | | X | | | | | X | | | | | X | |
| Concomitant | X | X | X | X | X | | X | | | X | | X | | | X | | X | | X | X | | X | X |
| Medications | | | | | | | | | | | | | | | | | | | | | | | |

Abbreviations: CSSRS: Columbia-Suicide Severity Rating Scale; DOA: Drugs of abuse; EOS: End of Study; ET: Early Termination; FSH: Follicle stimulating hormone; OP: out-patient; OV: out-patient visit; SU: study unit; TC: telephone call

Informed consent may be obtained prior to Study Visit 1
b. All inclusion/exclusion criteria should be assessed during screening and confirmed prior to randomization (Study Day −1).
c. Height and weight will be measured with shoes off during Screening. BMI will be calculated during Screening. Weight only (with shoes off) will be collected at Study Day −1 and Study Day 20 (EOS) and at Early Termination (ET).
d. A full physical examination will be performed during Screening and at Study Day 20 (EOS) and at Early Termination (ET). Unscheduled symptom-directed physical examinations may be conducted at any time at the Investigator's discretion.
e. Vital signs will include the following assessments: blood pressure, pulse rate, respiratory rate and body temperature. Vital signs will be performed after the participant has been resting for at least 5 minutes. Body temperature will be assessed during Screening, after admission to the Study Unit on Study Day −1, prior to dosing with study drug on Study Day 1 and prior to collection of blood samples on Study Day 20 and at Early Termination. On all other occasions, blood pressure, pulse rate (heart rate) and respiratory rate will be obtained prior to collection of PK samples and 10 minutes after completion of PK sampling. At each designated timepoint, the assessment window is ±20 minutes.
f. Drug testing is for the following substances: amphetamines, barbiturates, benzodiazepines, cocaine, MDMA, methamphetamines, methadone, opiates, oxycodone, PCP, tricyclic antidepressants, and THC
g. Any female subject who does not meet the criteria for non-childbearing is considered to be of childbearing potential. For any suspected postmenopausal female (greater than 50 years of age with spontaneous amenorrhea for 12 months or longer), FSH will be measured (Serum FSH levels must be ≥40 mIU/mL for the participant to be considered postmenopausal). For all female subjects of childbearing potential, pregnancy tests will be performed. A serum pregnancy test will be performed during Screening. A urine β-hCG test will be performed on Study Day −1 and Study Day 20 (and at Early Termination). If the urine β-hCG test is reported as positive, the result will be confirmed with a serum pregnancy test.
h. Serology for HIV (antigen/antibody test), HBV (HBsAg) and HCV (HepC Ab)
i. After eligibility has been confirmed, the patient will be randomized to one of two treatments (HSA-IGF2-R61A or placebo) according to the randomization plan developed by the study statistician.
j. Adverse events will be monitored for individual subjects from the time of signing of the informed consent through their discharge from the study.
k. Telephone check-ins will be conducted on Study Day 13 and Study Day 17 to review concomitant medications and AEs.
l. Hematology, serum chemistry, coagulation assessments and urinalyses will be collected at various points during the study as noted above. Assessments are detailed in the accompanying table. Covid testing will performed during Screening and on Study Day −1; testing may be repeated at any time during the study at the Investigator's discretion. Samples will also be obtained with PK sampling on Study Days 5, 8, 10, 15, and 20 (End of Study) and at Early Termination. On Study Day 1, blood samples for laboratory assessments will be drawn in the fasted state prior to administration of study drug. eGFR will only be calculated at Screening using the CKD-EPI Creatinine-Cystatin C Age, Sex Equation (2021) [see Laboratory Tests and Assessments Description, below]
m. Standard (safety) digital ECGs will be performed during Screening and on Study Days −1 4, 5, 8, 10, 15 and 20 (End of Study) and at Early Termination. All standard (safety) digital ECGs will be performed in triplicate (approximately 1 minute apart) after the subject has been supine for ≥5 minutes. Additional 12-lead ECGs may be performed at any other time if clinically indicated. A hard copy of the ECG will be printed and signed by the investigator at the investigative site. Abnormal findings will be assessed by the investigator as either clinically significant (CS) or not clinically significant (NCS).
n. 24-hour Holter monitoring will be initiated prior to administration of study drug on Study Day 1. Digital ECGs will be extracted at times of PK sampling. At each time, a printed copy will be reviewed at the study site while an electronic copy will be transmitted to the central reading facility for review and formal analysis.
o. PK sampling (plasma) will be collected pre-dose and at 0.5, 1, 1.5, 2, 3, 4, 6, 8, 12, 16, 24, 36 and 48 hours (±10 minutes at each time point) after the start of administration of the respective study drugs. Subjects will then be discharged from the Study Unit and will return for outpatient visits at 96 (Study Day 5), 168 (Study Day 8), 216 (Study Day 10), 336 (Study Day 15) and 456 (Study Day 20) (±6 hours) hours for additional sampling.
q. Blood samples for biomarkers (see Assessment Description, below) will be obtained per the schedule of events. Processing for retention and analysis will be described in the Laboratory Manual for the study.
r. Blood samples for immunogenicity testing will be drawn on Study Day 1 (pre-dose, with PK sample) and with PK samples on Study Day 10, Study Day 15 and Study Day 20.
   NOTE: When multiple study procedures are scheduled at the same time point, the order of procedures should be as follows: perform ECG, obtain heart rate and blood pressure, and collect PK samples. At times when study procedures are scheduled simultaneously, PK blood samples must be collected within the designated windows at the scheduled time points; other procedures will be performed as close to the scheduled time point as possible. Table 19 lists Schedule of Activities (SoA)—MAD: Individual Subjects.

TABLE 19
Schedule of Activities (SoA)-MAD: Individual Subjects

| | Study Day | | | | | | | | | | | | | | |
|---|---|---|---|---|---|---|---|---|---|---|---|---|---|---|---|
| | −28 | −1 | 1 | 2 | 3 | 4 | 5 | 6 | 7 | 8 | 9 | 10 | 11 | 12 | 13 |
| | Visit Location | | | | | | | | | | | | | | |
| Assessment | OP | SU | SU | SU | SU | OP | OV | OP | SU | SU | SU | SU | OP | OV | OP |
| Informed consent a | X | | | | | | | | | | | | | | |
| Demography | X | | | | | | | | | | | | | | |
| Medical/surgical | X | | | | | | | | | | | | | | |
| history | | | | | | | | | | | | | | | |
| Inc/exc criteria b | X | X | | | | | | | | | | | | | |
| CSSRS | X | X | | | | | | | | | | | | | |
| Height and weight c | X | X | | | | | | | | | | | | | |
| Physical | X | X | | | | | | | | | | | | | |
| examination d | | | | | | | | | | | | | | | |
| Vital signs e | X | X | X | X | X | | X | | X | X | X | X | | X | |
| DOA screen f | X | X | | | | | | | | | | | | | |
| Pregnancy | X | X | | | | | | | | | | | | | |
| testing g | | | | | | | | | | | | | | | |
| Serology (HIV, | X | | | | | | | | | | | | | | |
| Hep B/C) h | | | | | | | | | | | | | | | |
| Randomization i | | X | | | | | | | | | | | | | |
| HSA-IGF2-R61A | | | X | | | | | | | X | | | | | |
| or placebo | | | | | | | | | | | | | | | |
| Adverse Events j | | X | X | X | X | X | X | | X | X | X | X | X | X | |
| Telephone Safety | | | | | | X | | | | | | | X | | |
| Call k | | | | | | | | | | | | | | | |
| Concomitant | X | X | X | X | X | X | X | | X | X | X | X | X | X | |
| medications | | | | | | | | | | | | | | | |
| Lab Studies (see | X | X | X | X | X | | X | | X | X | X | X | | X | |
| list) l | | | | | | | | | | | | | | | |
| Covid testing l | | | | | | | | | | | | | | | |
| Coagulation tests l | | X | | X | | | | | X | | X | | | | |
| Urinalysis l | X | X | | X | | | | | X | | X | | | | |
| GTT l | | X | | | | | | | | | | | | | |
| Insulin l | | X | | | | | | | | | | | | | |
| Hemoglobin A1C l | | X | | | | | | | | | | | | | |
| Lipid Profiles l | | X | | | | | | | | | | | | | |
| Insulin Sensitivity l | | X | | | | | | | | | | | | | |
| 12-lead ECG m | X | X | X | X | X | | X | | X | X | X | X | | X | |
| Holter Monitor n | | | X | X | | | | | | X | X | | | | |
| Pharmacokinetics o | | | X | X | X | | X | | | X | X | X | | X | |
| Pharmacodynamics q | | | | | | | | | | | | | | | |
| Biomarker-Target | | | X | | | | | | X | X | | | | | |
| Biomarker- | | | X | | | | | | X | X | | | | | |
| Muscle | | | | | | | | | | | | | | | |
| Biomarker- | | | X | | | | | | X | X | | | | | |
| Metabolism | | | | | | | | | | | | | | | |
| Immunogenicity r | | | X | | | | | | | X | | | | | |
| | Study Day | | | | | | | | | | | | | | |
| | | | | | | | 20- | | 23- | | 27- | | 30- | | |

| | 14 | 15 | 16 | 17 | 18 | 19 | 21 | 22 | 25 | 26 | 28 | 29 | 35 | 36 |
|---|---|---|---|---|---|---|---|---|---|---|---|---|---|---|
| | Visit Location | | | | | | | | | | | | | |
| Assessment | SU | SU | SU | SU | OP | OV | OP | OV | OP | OV | OP | OV | OP | ES |
| Informed consent a | | | | | | | | | | | | | | |
| Demography | | | | | | | | | | | | | | |
| Medical/surgical | | | | | | | | | | | | | | |
| history | | | | | | | | | | | | | | |
| Inc/exc criteria b | | | | | | | | | | | | | | |
| CSSRS | | X | | | | | | | | | | | | X |
| Height and weight c | X | | | | | | | | | | | | | X |
| Physical | | | | | | | | | | | | | | X |
| examination d | | | | | | | | | | | | | | |
| Vital signs e | X | X | X | X | | X | | X | | X | | X | | X |
| DOA screen f | | | | | | | | | | | | | | X |
| Pregnancy | | | | | | | | | | | | | | X |
| testing g | | | | | | | | | | | | | | |
| Serology (HIV, | | | | | | | | | | | | | | |
| Hep B/C) h | | | | | | | | | | | | | | |
| Randomization i | | | | | | | | | | | | | | |
| HSA-IGF2-R61A | | X | | | | | | | | | | | | |
| or placebo | | | | | | | | | | | | | | |
| Adverse Events j | X | X | X | X | X | X | | X | X | X | X | X | X | X |
| Telephone Safety | | | | | X | | | | X | | X | | X | |
| Call k | | | | | | | | | | | | | | |
| Concomitant | X | X | X | X | X | X | | X | X | X | X | X | X | X |
| medications | | | | | | | | | | | | | | |
| Lab Studies (see | X | X | X | X | | X | | X | | X | | X | | X |
| list) l | | | | | | | | | | | | | | |
| Covid testing l | | | | | | | | | | | | | | |
| Coagulation tests l | X | | X | | | | | | | | | | | X |
| Urinalysis l | X | | X | | | | | | | | | | | X |
| GTT l | X | | | | | | | | | | | | | X |
| Insulin l | X | | | | | | | | | | | | | X |
| Hemoglobin A1C l | X | | | | | | | | | | | | | X |
| Lipid Profiles l | X | | | | | | | X | | | | | | X |
| Insulin Sensitivity l | X | | | | | | | | | | | | | X |
| 12-lead ECG m | X | X | X | X | | X | | X | | X | | X | | X |
| Holter Monitor n | | X | X | | | | | | | | | | | |
| Pharmacokinetics o | | X | X | X | | X | | X | | X | | X | | X |
| Pharmacodynamics q | | | | | | | | | | | | | | |
| Biomarker-Target | X | X | | X | | | | X | | | | X | | X |
| Biomarker- | X | X | | X | | | | X | | | | X | | X |
| Muscle | | | | | | | | | | | | | | |
| Biomarker- | X | X | | X | | | | X | | | | X | | X |
| Metabolism | | | | | | | | | | | | | | |
| Immunogenicity r | | X | | | | | | X | | | | | | X |

Abbreviations: CSSRS: Columbia-Suicide Severity Rating Scale; DOA: Drugs of abuse; EOS: End of Study; ET: Early Termination FSH: Follicle stimulating hormone; OP: out-patient; OV: out-patient visit; SU: study unit; TC: telephone call

Informed consent may be obtained prior to Study Visit 1
b. All inclusion/exclusion criteria should be assessed during screening and confirmed prior to randomization (Study Day −1).
c. Height and weight will be measured with shoes off during Screening. BMJ will be calculated during Screening. Weight only (with shoes off) will be collected at Study Day −1 and Study Day 36 (EGS) and at Early Termination (ET).
d. A full physical examination will be performed during Screening and on Study Day 36 (EGS) and at Early Termination (ET). Unscheduled symptom-directed physical examinations may be conducted at any time at the Investigator's discretion.
e. Vital signs will include the following assessments: blood pressure, pulse rate, respiratory rate and body temperature. Vital signs will be performed after the participant has been resting for at least 5 minutes. Body temperature will be assessed during Screening, after admission to the Study Unit on Study Day −1, prior to dosing with study drug on Study Day 1 and prior to collection of blood samples on Study Day 36 and at Early Termination. On all other occasions, blood pressure, pulse rate (heart rate) and respiratory rate will be obtained prior to collection of PK samples and 10 minutes after completion of PK sampling. At each designated timepoint, the assessment window is ±20 minutes.
f. Drug testing is for the following substances: amphetamines, barbiturates, benzodiazepines, cocaine, MDMA, methamphetamines, methadone, opiates, oxycodone, PCP, tricyclic antidepressants, and THC
g. Any female subject who does not meet the criteria for non-childbearing is considered to be of childbearing potential. For any suspected postmenopausal female (greater than 50 years of age with spontaneous amenorrhea for 12 months or longer), FSH will be measured (Serum FSH levels must be ≥40 mIU/mL for the participant to be considered postmenopausal). For all female subjects of childbearing potential, pregnancy tests will be performed. A serum pregnancy test will be performed during Screening. A urine β-hCG test will be performed on Study Day −1 and Study Day 36 (and at Early Termination). If the urine β-hCG test is reported as positive, the result will be confirmed with a serum pregnancy test.
h. Serology for HIV (antigen/antibody test), HBV (HBsAg) and HCV (HepC Ab)
i. After eligibility has been confirmed, the patient will be randomized to one of two treatments (HSA-IGF2-R61A or placebo) according to the randomization plan developed by the study statistician.
j. Adverse events will be monitored for individual subjects from the time of signing of the informed consent through their discharge from the study.
k. Telephone check-ins will be conducted on Study Days 4, 11, 18, 24, 26, 32 to review concomitant medications and AEs.
l. Hematology, serum chemistry, coagulation assessments and urinalyses will be collected at various points during the study as noted above. Assessments are detailed in the accompanying table. Covid testing will performed during Screening and on Study Day −1; testing may be repeated at any time during the study at the Investigator's discretion. Samples will also be obtained with PK sampling on Study Days 3, 5, 7, 9, 10, 12, 14, 16, 17, 19, 22, 26, 29 and 36 (End of Study) and at Early Termination. On Study Days 1, 8 and 15, blood samples for laboratory assessments will be drawn in the fasted state prior to administration of study drug. eGFR will only be calculated at Screening using the CKD-EPI Creatinine-Cystatin C Age, Sex Equation (2021) [see Laboratory Tests and Assessments Description, below]
m. Standard (safety) digital ECGs will be performed during Screening and on Study Days −1, 3, 5, 7, 10, 12, 14, 17, 19, 22, 26, 29 and 36 (End of Study) and at Early Termination. All standard (safety) digital ECGs will be performed in triplicate (approximately 1 minute apart) after the subject has been supine for ≥5 minutes. Additional 12-lead ECGs may be performed at any other time if clinically indicated. A hard copy of the ECG will be printed and signed by the investigator at the investigative site. Abnormal findings will be assessed by the investigator as either clinically significant (CS) or not clinically significant (NCS).
n. 24-hour Holter monitoring will be initiated prior to administration of study drug on Study Day 1. Digital ECGs will be extracted at times of PK sampling. At each time, a printed copy will be reviewed at the study site while an electronic copy will be transmitted to the central reading facility for review and formal analysis.
o. Study Drug will be administered subcutaneously on Study Day 1, Study Day 8 and Study Day 15. On these days, PK sampling (plasma) will be collected pre-dose and at 0.5, 1, 1.5, 2, 3, 4, 6, 8, 12, 16, 24, 36 and 48 hours (±10 minutes at each time point) after the start of administration of the respective study drugs. Subjects will then be discharged from the Study Unit. Subjects will return for outpatient visits on Study Days 5, 12, 19, 22, 26, 29 and 36) (±6 hours) hours for additional sampling. These samples will be drawn in a fasting state.
q. Blood samples for biomarkers (Target, Muscle, Metabolism; see Assessment Description, below) will be obtained per the schedule of events. Processing for retention and analysis will be described in the Laboratory Manual for the study.
r. Blood samples for immunogenicity testing will be drawn on Study Day 1 (pre-dose, with PK sample) and with PK samples on Study Days 8, 15, 22 and 36 (EoS).

NOTE: When multiple study procedures are scheduled at the same time point, the order of

procedures should be as follows: perform ECG, obtain heart rate and blood pressure, and collect PK samples. At times when study procedures are scheduled simultaneously, PK blood samples must be collected within the designated windows at the scheduled time points; other procedures are performed as close to the scheduled time point as possible.

### Laboratory Tests

Clinical laboratory evaluations are performed centrally. Decisions regarding eligibility for this study may be made using local laboratory determinations. For dosing decisions, local hematology and chemistry laboratory results may be used. Handling and shipment of clinical laboratory samples is outlined in the study manual.

Clinical laboratory evaluations are performed as outlined below in Table 20. Blood samples for analysis of the following clinical chemistry and hematological parameters are obtained as specified in the Schedule of Events.

TABLE 20
Clinical laboratory evaluations

| | | |
|---|---|---|
| Hematology | Hemoglobin | MCH |
| | Hematocrit | MCHC |
| | RBC count | MCV |
| | Platelet (count) | WBC count with differential (absolute & %) |
| Serum Chemistry | Sodium | Total and indirect bilirubin |
| | Potassium | Alkaline phosphatase |
| | Chloride | ALT |
| | Bicarbonate | AST |
| | Glucose | Lactate dehydrogenase (LDH) |
| | Calcium | Gamma glutamyl transferase (GGT) |
| | Creatinine | Inorganic phosphate |
| | Blood urea nitrogen | Magnesium |
| | (BUN) | Creatine phosphokinase (CPK) |
| | Total protein | |
| | Albumin | |
| Glucose | Hemoglobin A1C | Insulin |
| metabolism & | C-peptide | Insulin resistance (HOMA-IR, QUICKI) |
| insulin sensitivity | | |
| Coagulation | PT | INR |
| | PTT | |
| Urinalysis | Specific gravity | Urine blood |
| | pH | Urine ketones |
| | Leukocyte esterase | Urine bilirubin |
| | Urine protein | Urine glucose |
| Lipid levels | Total cholesterol | HDL-C |
| | LDL-C | triglycerides |
| Screening Serology | HIV (antigen/antibody) | HCV (HepC Ab) |
| | HBV (HBsAg) | |
| Immunogenicity | Anti-drug antibodies | Neutralizing antibodies |
| Other | Cystatin C | eGFR: CKD-EPI Creatinine-Cystatin C Age, Sex |
| | | Equation (2021) [See below] |
| Cardiovascular | Echocardiogram | Safety ECG |
| | | Holter Monitoring with ECG extractions |

### CKD-EPI Creatinine-Cystatin C Age, Sex Equation (2021)

$$eGFR = 135 \times {min ( Scr / k , 1 )}^{α} \times {max ( Scr / k , 1 )}^{- 0.544} \times {min ( S cys / 0.8 , 1 )}^{- 0.323} \times {max ( S cys / 0.8 , 1 )}^{- 0.778} \times {0.9961}^{age} \times 0.963 [ if female ]$$

where
Scr=serum creatinine in mg/dL,
Scys is serum cystatin C in mg/dL,
k is 0.7 for females and 0.9 males,
α is −0.219 for females and −0.144 for males,
min indicates the minimum of Scr/k or 1,
   max indicates the maximum of Scr/k or 1]

TABLE 21
Assessments - Description

| Assessment | Description |
|---|---|
| Columbia-SSRS | The Columbia Protocol, also known as the Columbia-Suicide Severity Rating |
| | Scale (C-SSRS), supports suicide risk assessment through a series of simple, |
| | plain-language questions that anyone can ask. The answers help users identify |
| | whether someone is at risk for suicide, assess the severity and immediacy of |
| | that risk, and gauge the level of support that the person needs. |
| 12-lead ECG & Holter | A 12-lead, 24-hour Holter recorder is used to collect safety ECGs, at times |
| monitor | designated in the Schedule of Assessments, and to provide 24-hour |
| | arrhythmia monitoring. Safety ECGs are “captured” at the appropriate time. |
| | They are transmitted to a central reading facility. Copies are printed for local |
| | review. |
| Pharmacokinetics | PK determinations include at least Cₘₐₓ, Tₘₐₓ, estimated terminal phase half- |
| | life in plasma (t_{1/2λz}); area under the concentration-time curve (AUC) from |
| | time 0 to 24 h after administration (AUC_{0-24 h}) and percent dose excreted in |
| | urine. |
| Glucose Tolerance Test | A 2-hour oral glucose tolerance test (GTT) is performed under fasted |
| (Glucose and insulin levels) | conditions. Participants are administered 75 grams (g) glucose solution prior |
| | to administration of the first dose of study drug and on Study Day 15. Glucose |
| | and insulin levels are obtained at each time point. |
| Lipid Profiles | Total Cholesterol Level; Low-density Lipoproteins (LDL-C) Level; High- |
| | density Lipoproteins (HDL-C) Level; Triglycerides Level |
| Insulin Sensitivity | QUICKI |
| | The QUICKI is based on fasting glucose and insulin measurements and are |
| | calculated using the following equation: |
| | QUICKI = 1/[log(fasting glucose in mg/dL) + log (fasting insulin in |
| | uU/mL)] |
| | ISI-MATSUDA |
| | The ISI-Matsuda is based on the average glucose and insulin values obtained |
| | during the entire oral glucose tolerance test and are calculated using the |
| | following equation: |
| | ISI = 10,000/√[fasting glucose (mg/dL) × fasting insulin(uU/mL) × mean |
| | glucose × mean insulin] |
| Biomarker-Muscle | mRNA and microRNA expression changes, in particular, expression of |
| | candidate microRNAs relevant to the following targets: insulin receptor, |
| | muscle chloride channel, SERCA1, RyR1 and troponin T. The following |
| | microRNA targets may also be assessed: miR-1, miR-133b, miR-29 and miR- |
| | 206. |
| Biomarker-Target | IGF-1. Other markers may be identified and included in testing. |
| Biomarker-Metabolism | Glucose, insulin, insulin resistance, GTT, lipids, glucagon |

The results of this Example indicate that administration of HSA-IGF2-R61A has acceptable levels of safety and tolerability in healthy volunteers, with minimal risks, and merits proceeding to further clinical testing of the compound.

### Example 10—Phase 2 Proof-of-Concept Dose-Ranging Study in Human Patients with DM1

Approximately 60 subjects with adult-onset clinically demonstratable DM1 are enrolled and treated with study drug (HSA-IGF2-R61A or placebo) for 12 weeks. Endpoints evaluated include those related to function (mobility, strength), muscle mass, pulmonary function, patient reported outcomes, cognitive function, metabolic markers, quality of life/activities of daily living, biomarkers associated with target engagement and myogenesis, in addition to monitoring safety and tolerability.

### Study Objectives and Endpoints

### Primary:

In patients with adult-onset myotonic dystrophy type 1 to compare subcutaneous administration of HSA-IGF2-R61A to placebo over 12 weeks with regard to objectives and endpoints in Table 22.

TABLE 22
Primary objectives and endpoints

| Study Objectives | Endpoints |
|---|---|
| Efficacy as changes | Change from baseline to Week 12 as assessed |
| in motor strength | by grip strength (force) compared to: |
| | Baseline |
| | Placebo |
| | Age- and severity-matched controls from the END- |
| | DM1 database (if available) |

Secondary: In patients with adult-onset myotonic dystrophy type 1 to compare subcutaneous administration of HSA-IGF2-R61A to placebo over 12 weeks with regard to objectives and endpoints in Table 23.

TABLE 23
Secondary objectives and endpoints

| Study Objectives | Endpoints |
|---|---|
| Safety and | The proportion of participants with adverse events, |
| tolerability | serious adverse events (SAEs) and drug-related |
| | adverse events |
| Efficacy as changes | Change from baseline to Week 12 as assessed by |
| in activity and | DM1-Activ-C (score) compared to: |
| impact | Baseline |
| | Placebo |
| | Age- and severity-matched controls from the END- |
| | DM1 database (if available) |
| Efficacy as changes | Change from baseline to Week 12 as assessed by |
| in activity and | myotonic dystrophy health index (score) compared |
| impact | to: |
| | Baseline |
| | Placebo |
| | Age- and severity-matched controls from the END- |
| | DM1 database (if available) |
| Efficacy as changes | Change from baseline to Week 12 as assessed by |
| in motor strength | Qualitative Muscle Testing score compared to: |
| | Baseline |
| | Placebo |
| | Age- and severity-matched controls from the END- |
| | DM1 database (if available) |
| Efficacy as changes | Change from baseline to Week 12 as assessed by |
| in metabolic profile | glucose, insulin, hemoglobin A1c, C peptide and |
| (including insulin | calculated insulin sensitivity compared to: |
| sensitivity) | Baseline |
| | Placebo |
| | Age- and severity-matched controls from the END- |
| | DM1 database (if available) |
| Efficacy as changes | Change from baseline to Week 12 as assessed by |
| in perceived | Clinical Global Impression-Improvement compared |
| clinical benefit | to: |
| (clinician) | Baseline |
| | Placebo |
| Efficacy as changes | Change from baseline to Week 12 as assessed by |
| in perceived clinical | Clinical Global Impression-Improvement compared |
| benefit (patient/ | to: |
| caregiver) | Baseline |
| | Placebo |
| Efficacy as changes | Change from baseline to Week 12 as assessed by |
| in motor function. | Timed Up-and-Go (TUG) test in seconds compared |
| | to: |
| | Baseline |
| | Placebo |
| | Age- and severity-matched controls from the END- |
| | DM1 database (if available) |
| Efficacy as changes | Change from baseline to Week 12 as assessed by |
| in motor function. | 10-meter walk time in seconds compared to: |
| | Baseline |
| | Placebo |
| | Age- and severity-matched controls from the END- |
| | DM1 database (if available) |
| Efficacy as changes | Change from baseline to Week 12 as assessed by the |
| in quality of life | individualized neuromuscular quality of life (Score) |
| | compared to: |
| | Baseline |
| | Placebo |
| | Age- and severity-matched controls from the END- |
| | DM1 database (if available) |

To understand the pharmacokinetic profile of HSA-IGF2-R61A in patients with adult-onset myotonic dystrophy type 1:

| Study Objectives | Endpoints |
|---|---|
| Following weekly | Define the acute and steady state PK profile for HSA- |
| administration of | IGF2-R61A in patients (including, but not limited to): |
| HSA-IGF2-R61A | Time to maximum concentration (Tₘₐₓ) |
| over 12 weeks | Maximum observed concentration (Cₘₐₓ) |
| | Area under the curve from time zero to infinity |
| | (AUC_{(0-∞)}) |
| | Area under the curve from time zero to last |
| | measurable concentration (AUC(₀₋ₗₐₛₜ₎) |
| | Terminal half-life (t_{1/2}) |
| | Steady-state concentrations |

### Exploratory:

In patients with adult-onset myotonic dystrophy type 1 to compare subcutaneous administration of HSA-IGF2-R61A to placebo over 12 weeks with regard to exploratory study objections and endpoints in Table 24.

TABLE 24
Exploratory study objections and endpoints

| Study Objectives | Endpoints |
|---|---|
| Safety assessed | Change from baseline to Week 12 and through the |
| by immunogenicity | Follow-up period as assessed by presence (and |
| | titers) of anti-drug antibodies (ADAs) and |
| | identification of neutralizing antibodies (Nabs) |
| Efficacy as changes | Change from baseline to Week 12 as assessed by the |
| in lean body | DEXA Scan compared to: |
| mass | Baseline |
| | Placebo |
| | Age- and severity-matched controls from the END- |
| | DM1 database (if available) |
| Efficacy as changes | Change from baseline to Week 12 as assessed by |
| in myotonia | video hand opening time (vHOT) compared to: |
| | Baseline |
| | Placebo |
| | Age- and severity-matched controls from the END- |
| | DM1 database (if available) |
| Efficacy as changes | Change from baseline to Week 12 as assessed by |
| in pulmonary | forced expiratory volume in 1 second (FEV1) and |
| function | forced vital capacity (FVC) obtained through |
| | spirometry compared to: |
| | Baseline |
| | Placebo |
| | Age- and severity-matched controls from the END- |
| | DM1 database (if available) |
| Efficacy as changes | Change from baseline to Week 12 as assessed by |
| in sleepiness | Epworth Sleepiness Scale (score) compared to: |
| | Baseline |
| | Placebo |
| | Age- and severity-matched controls from the END- |
| | DM1 database (if available) |
| Efficacy as changes | Change from baseline to Week 12 as assessed by |
| in fatigue | Fatigue Severity Scale (score) compared to: |
| | Baseline |
| | Placebo |
| | Age- and severity-matched controls from the END- |
| | DM1 database (if available) |
| Pharmacodynamic | Change from baseline to Week 12 as assessed by |
| effects | Biomarkers related to target engagement |
| | Biomarkers related to metabolism |
| | Biomarkers related to myogenesis |
| | compared to: |
| | Baseline |
| | Placebo |

### Study Rationale

The present study (HSA-IGF2-R61A-201) aims to assess the efficacy, safety, tolerability, and pharmacokinetics (PK) of HSA-IGF2-R61A administered subcutaneously to patients with DM1.

This study is designed to support future clinical development of HSA-IGF2-R61A in DM1 and in other degenerative myopathies and other disorders for which preclinical efficacy data have been obtained.

### Trial Design

HSA-IGF2-R61A-201 is a multi-center, randomized, double-blind, placebo-controlled assessment of the efficacy, safety, pharmacokinetics (PK) and pharmacodynamics (PD) of HSA-IGF2-R61A in patients with symptomatic adult-onset DM1.

This is the first study of HSA-IGF2-R61A in patients. Goals of the study are to evaluate safety and tolerability of HSA-IGF2-R61A in patients with DM1, determine dose(s) of HSA-IGF2-R61A to include in a pivotal study in the subject population and to define more clearly clinically-meaningful endpoints representing symptomatic improvement of symptomatic patients with adult-onset DM1.

Doses of HSA-IGF2-R61A administered in this study are determined following review of results from non-clinical toxicology and the first-in-human study in healthy normal volunteers (HSA-IGF2-R61A-101). As place holders, doses of HSA-IGF2-R61A administered in this study are designated as HSA-IGF2-R61A-LO and HSA-IGF2-R61A-HI.

The study includes 3 periods lasting approximately 28 weeks:
Screening Period: Up to 4 weeks
Treatment Period: 12 weeks
Follow-up Period: 12 weeks

Subjects are screened within 28 days of dosing. During this period, subjects provide informed consent and undergo studies to ensure they meet inclusion and exclusion criteria. See the Schedule of Assessments for assessments to be performed during the Screening Period.

On Study Day −1, eligible subjects attend the clinical study site. After confirming eligibility criteria and obtaining baseline functional and laboratory assessments, subjects undergo an oral glucose tolerance test (GTT). Several functional and questionnaire assessments are also performed on this day. See the Schedule of Assessments for a detailed listing.

Subsequently, subjects are randomized into treatment Cohorts.

In a sub-study, 12 randomized subjects (following procedure-specific informed consent) undergo a baseline quadriceps needle muscle biopsy on the subject's dominant side. Approximately 4 subjects from each Cohort (HSA-IGF2-R61A-HI, HSA-IGF2-R61A-LO, placebo) participate in the sub-study.

On Study Day 1, subjects receive study drug in a fed state. (Because of the mechanism of action of HSA-IGF2-R61A and the potential for hypoglycemia, all doses of study drug are to be administered in the fed state.) Subjects undergo pharmacokinetic (PK) sampling according to the PK sampling schedule specified in the protocol through 24 hours. At 4, 6, 12 and 24 hours post dosing, glucose sampling is performed.

Subjects remain in the study facility for at least 24 hours following the first administration of study drug.

Subjects return to the study site on Study Day 5 and at Week 2, Week 3, Week 4, Week 6, Week 9 and Week 12 during the Treatment Period. Subjects also return at Study Week 13, Week 18 and Week 24 during the Follow-up Period. Site staff contacts subjects via telephone on Study Week 5, Week 7, Week 8, Week 10, Week 11 during the Treatment period and on Study Week 15 and Week 21 during the Follow-up period. During the telephone calls site staff inquiries about the subject's general status and ask specifically about concomitant medications and potential adverse events.

At all site visits, vital signs are obtained and subjects are queried about adverse events and concomitant medications. Laboratory studies (hematology, chemistry, coagulation, immunogenicity, urinalysis) are obtained at visits during the Treatment Period at Week1, Week 2, Week 3, Week 6, Week 9 and Week 12. During the Follow-up Period, laboratory tests are obtained at Week 15, Week 21 and Week 24. (See Schedule of Assessments.)

Assessments of glucose and insulin metabolism (e.g., glucose, insulin, Hemoglobin A1C and calculations of insulin sensitivity/resistance) is performed on samples taken on Day 1 and at Week 6 and Week 12 (during the Treatment Period) and at the End of Study visit at Week 24.

At the Week 15 visit (during Follow-up), a repeat muscle biopsy is obtained.

Efficacy Assessments (walking, myotonia, muscle strength, fine motor function, other symptom-related assessments and patient-reported outcomes) are assessed at baseline (Study Day −1 or Study Day 1), during treatment (Weeks 6 and 12) and at the end of study (Week 24).

A Data Safety Monitoring Board (DSMB) reviews available PK data, laboratory data and clinical observations, including adverse events, periodically during the study. Reviews occur after at least:
3 patients have completed their initial dosing visits,
12 patients have completed their initial dosing visits and
12 patients have completed their Month 3 dosing visit.

Additional reviews may occur as deemed necessary by the DSMB.

### Cohorts:

Approximately 60 subjects are randomized (1:1:1) into 3 study cohorts across 6-8 clinical study centers:
HSA-IGF2-R61A-LO (n=20)
HSA-IGF2-R61A-HI (n=20)
Placebo (n=20)

Study-related activities are described below. See Schedule of Activities.

### Subject Population

Subjects are males or females, ages 18 to 55, inclusive, with symptomatic adult-onset DM1.

For enrollment, there are no limitations with regard to gender, ethnicity or racial background. However, DM1 is a rare disease and affects races and ethnicities differently; notably, DM1 is particularly uncommon in Asian and Black populations.

### Dose & Dose Justification

Two dose levels of HSA-IGF2-R61A, here designated LO and HI, are used for this study. Each subject receives a single subcutaneous dose of study drug (HSA-IGF2-R61A-LO; HSA-IGF2-R61A-HI; placebo) on Study Day 1 and then weekly for the following 11 weeks. The Follow-up period lasts an additional 12 Weeks.

The dose selection of HSA-IGF2-R61A is based on results from final toxicology studies and data from Study HSA-IGF2-R61A-101 (Combined single- and multiple-ascending multiple dose study in healthy volunteers). It is expected that both doses of HSA-IGF2-R61A employed in this protocol, HSA-IGF2-R61A-201, show evidence of efficacy and are expected to be well tolerated.

### Route of Delivery

Doses are administered subcutaneously (SC).

Site personnel administer study drug on Study Day 1 and at the Week 2 visit. During these visits, participants and/or caregivers are instructed on care of study drug and procedures for administration. During the Week 3 and Week 4 study visits, the participant or caregiver administers study drug under the supervision of site personnel. Participants or caregivers administer study drug via subcutaneous injection (abdomen, thigh, deltoid regions) on designated days during Study Weeks 7, 9, 10 and 11. At site visits for Week 6, Week 9 and Week 12, the participant or caregiver administers study drug under the supervision of site personnel.

### Eligibility Criteria—Inclusion Criteria:

Subjects must meet all the following criteria at the time of enrollment:
1. Are males or nonpregnant females, ages 18 to 60 (inclusive) at time of signing Informed Consent with body mass index (BMI) 18 to 35 kg/m²
2. Have provided a written informed consent (signed and dated) and any authorizations required by local law
3. Are able to understand the requirements of the study protocol and are willing to comply with all study requirements
4. Have a clinical diagnosis of symptomatic myotonic dystrophy type 1 with onset after the age of 10 years
5. Have genetic confirmation of DM1 with dystrophia myotonica-protein kinase (DMPK) CTG repeat length ≥100
6. Have sufficient finger flexor strength to grasp the handle of the ergometer used to measure myotonia
7. Are ambulatory (orthoses allowed, canes and walkers not allowed) and able to walk at least 25 meters at Screening
8. Have negative screens for alcohol and drugs of abuse at screening and admission
9. Females must be either:
   of non-childbearing potential (defined as having undergone surgical sterilization (hysterectomy, bilateral salpingectomy, bilateral oophorectomy) or being postmenopausal (i.e., greater than 45 years old with amenorrhea for ≥12 months AND a serum follicle stimulating hormone (FSH) level >40 mIU/mL)
OR
   of child-bearing potential and using at least one of the following acceptable methods of contraception from the time of informed consent through the study Follow-up Visit or at least 8 weeks after the last dose of Study Drug:
   a. Stable use of hormonal contraceptive treatment (including oral, intravaginal or transdermal combined (estrogen- and progestogen-containing) contraception, oral or injectable progestogen-only contraception, and intrauterine hormone-releasing systems)
   b. Intrauterine device (IUD, including hormone-releasing IUDs)
   c. Monogamous relationship with a vasectomized partner
   d. Sexual abstinence (i.e., refraining from heterosexual intercourse) for 8 weeks prior to signing informed consent
10. Men who are sexually active must use the following forms of medically acceptable birth control during the study drug treatment period and for 16 weeks after the last administration of study drug:
   a. Vasectomy with medical assessment of surgical success
   OR
   b. Consistent use of a condom with partner also using either stable hormonal contraceptive or IUD.
   c. Sperm donation is prohibited during the study and for up to 4 weeks after the last administration of study drug.
11. Have NOT participated in a clinical study utilizing an investigational agent within 28 days or within 6 half-lives of the investigational drug (whichever is longer) prior to Screening.

### Eligibility Criteria—Exclusion Criteria

In order to participate in the study, the participant must meet NONE of the following at the time of enrollment:
1. Be unwilling or unable to comply with study procedures (e.g., muscle biopsies), including follow-up, as specified by the protocol, or unwillingness to cooperate fully with the Investigator
2. Have a history of non-compliance with other therapies
3. Have a history of drug or alcohol abuse within 3 months of Screening
4. Have evidence of clinically significant abnormalities or disease, including, the following:
   a. Impaired renal function (estimated glomerular filtration rate [eGFR]<60 ml/min/1.73 m², using the CKD-EPI (CysC) equation)
   b. Urine dipstick results
      > trace protein; if present, a quantitative total urine protein measurement of <0.5 g/24 hours would enable study enrollment
      > trace blood; if present, urine microscopy showing <5 red blood cells per high power field would enable study enrollment
   c. Alanine aminotransferase (ALT) or aspartate aminotransferase (AST)>3.0×the upper limit of normal (ULN).
   d. Bilirubin >ULN (unless has a genetically confirmed diagnosis of Gilbert's syndrome)
   e. Platelet count <125×10⁹/L
   f. Electrocardiogram (ECG) showing second- or third-degree AV block, atrial flutter, atrial fibrillation, ventricular arrhythmias, or QTcF>470 msec female or >450 msec male
   g. Left ventricular ejection fraction <35% on a transthoracic, 3-dimensional echocardiogram.
   h. Any other laboratory, vital sign, ECG abnormality, or clinical history or finding that, in the investigator's opinion, is likely to increase the risk of study participation, confound study results, or interfere with study conduct or adherence
6. Have an active malignancy or have a history of malignancy within the 5 years prior to Screening (subjects with pilomatricoma or prior basal or squamous cell carcinoma of the skin or carcinoma in situ of the cervix that has been successfully treated may be enrolled)
7. Have received treatment with
   a. another investigational drug, biologic agent, or device within 1 month of Screening, or 5 half-lives of investigational agent, whichever was longer
   b. systemic corticosteroids within 8 weeks prior to the first dose of Study Drug
   c. an anti-myotonia medication within 30 days prior to Screening; may have included, but were not limited to: phenytoin, carbamazepine, procainamide, disopyramide, nifedipine, acetazolamide, clomipramine, imipramine, amitriptyline, taurine, quinine, mexiletine
   d. medication for cardiac arrhythmia
8. If being treated with testosterone for hypogonadism, is on a stable replacement dose
9. Have these active medical conditions or any clinically significant laboratory abnormality, medical or psychiatric illness which, in the opinion of the Investigator, could interfere with the conduct or interpretation of the study or put the participant at risk
   a. Uncontrolled diabetes mellitus (Hemoglobin A1c≥6.5%)
   b. Symptomatic coronary artery disease, cardiomyopathy, congestive heart failure
   c. Implanted device for the treatment of cardiac arrhythmias (i.e., pacemaker or defibrillator)
   d. Untreated thyroid dysfunction
   e. Seizure disorder
   f. Arthritis or other medical condition (except DM1) that would limit ambulation
   g. Bleeding disorder or history of excessive bleeding
10. Have any of the following known active infections:
   a. Infection requiring systemic antiviral or antimicrobial therapy that would not have been completed prior to Study Day 1
   b. HIV not optimally controlled or treated. Participants with HIV who are on sustained stable antiretrovirals (for >4 weeks) and have CD4+ counts ≥350 cells/μL may be enrolled. No HIV testing is required unless mandated by local health authority.
   c. Chronic hepatitis B virus (HBV) infection with surface antigen positive: subjects with a prior history of treated HBV infection who are hepatitis B surface antigen-negative may be enrolled. No testing is required for hepatitis B unless mandated by local health authority.
   d. Chronic hepatitis C virus (HCV) infection: untreated or on active treatment. Participants with a prior history of treated HCV infection who are HCV RNA-undetectable may be enrolled. No testing is required for hepatitis C unless mandated by local health authority.
11. Have a history of hypersensitivity to local anesthetics that were to be used in the biopsy procedure or components thereof
12. Prior exposure to HSA-IGF2-R61A
13. History or immune reaction to any biologic therapy
14. Donation or loss of greater than 1 unit (450 mL) of blood within 1 month prior to dosing
   Additional exclusion criteria for subjects participating in the muscle biopsy sub-study include:
15. Use of anticoagulant such as warfarin or a direct oral anticoagulant (e.g., dabigatran) due to the increased risk of bleeding.
16. Use of aspirin or non-steroidal anti-inflammatory agents should be discontinued 3 days prior to the biopsy procedure, if possible.
17. Platelet count <50,000 (if known) due to the increased risk of bleeding.
18. History of a bleeding disorder due to the increased risk of bleeding.
19. Advanced wasting of tibialis anterior (TA) muscle that precludes needle muscle biopsy in order to ensure that a sample taken would be of muscle and not just fat and fascia.
20. Previous muscle biopsy of either TA in order to provide muscle tissue samples of non-biopsied muscles.

### Trial Enrollment Targets

The goal for this study is to enroll approximately 60 (males and females) with symptomatic adult-onset myotonic dystrophy type 1. Sample size for this study is consistent with exploratory Phase 2a studies.

This study attempts to enroll a representative sample of the general population as noted in the table below. The disease being studied is confined to adults, so no pediatric subjects are entered. Targeted/planned enrollment is listed in Table 25.

TABLE 25
Targeted/planned enrollment
TARGETED/PLANNED ENROLLMENT: Number of Subjects

| | Sex/Gender | | |
|---|---|---|---|
| | Females | Males | Total |
| Ethnic Category | | | |
| Hispanic or Latino | 2 | 2 | 4 |
| Not Hispanic or Latino | 28 | 28 | 56 |
| Ethnic Categories: TOTAL | 30 | 30 | 60 |
| Racial Categories | | | |
| American Indian/Alaska Native | 0 | 0 | 0 |
| Asian | 2 | 1 | 3 |
| Native Hawaiian or Other Pacific Islander | 0 | 0 | 0 |
| Black or African American | 4 | 4 | 8 |
| White | 24 | 25 | 49 |
| Racial Categories: TOTALS | 30 | 30 | 60 |

### Duration

Total duration of subject participation in the study is up to 28 weeks.
The Screening Period for the study is up to 28 days (Including Day −1).
The Treatment Period is 12 weeks. Study drug (HSA-IGF2-R61A or placebo) is administered on Day 1 and then weekly for a total of 12 doses. Subjects are observed for safety for 24 hours in the CRU following the first dose.
The Follow-up period lasts 12 weeks
   Schedule of Evaluations—Screening (Up to 28 day)

Subjects undergo informed consenting procedures. Initial evaluations of demography, medical history, inclusion/exclusion criteria, height and weight (with calculation of BMI), physical examination, vital signs, 12-lead ECG, echocardiogram, urinalysis, FSH (to document menopause, as appropriate), screen for drugs of abuse, HIV, Hepatitis B & C and laboratory tests (chemistry, hematology and coagulation) are performed.

For familiarization purposes, subjects complete functional walking assessments (10-Meter Walk/Run time, Timed Up and Go), functional strength assessments (quantitative muscle testing, muscular impairment rating scale), symptom-related assessments (spirometry), patient activity impact assessments (DM1-Activ-C, MDHI) and patient-reported outcome assessments (individualized neuromuscular quality of life, lower extremity functional scale). Scores for these assessments are recorded but are not included in statistical analyses.

Subjects complete the Columbia-Suicide Severity Rating Scale during the Screening Period.

### Study Day −1

Subjects are seen at the clinical trial site. Inclusion/exclusion criteria is reviewed. Evaluations of body weight, physical examination, vital signs, 12-lead ECG and a screen for drugs of abuse are performed. Laboratory studies (hematology, chemistry, coagulation, urinalysis) are obtained to ensure inclusion/exclusion criteria continue to be met. Subjects are randomized to study drug (HSA-IGF2-R61A-LO, HSA-IGF2-R61A-HI, placebo) in a 1:1:1 ratio. Adverse events and concomitant medications from the time of signing the informed consent are reviewed. A needle biopsy of the quadriceps muscle for PD is obtained for subjects participating in the sub-study.

### Study Day 1 and Study Day 2

Baseline values for vital signs, 12-lead ECG and laboratory studies (hematology, chemistry, coagulation, urinalysis) are collected. A Holter monitor is applied prior to dosing and records for at least 24 hours post-dosing. Before and following administration of study drug, blood samples are obtained for pharmacokinetics (PK) and pharmacodynamics (PD); similarly, urinalysis samples are obtained for PD. Information regarding concomitant medication is reviewed. Subjects are monitored for adverse events.

As noted above, subjects receive study drug in the fed state. Subjects are observed at the study center for at least 24 hours following administration of the first dose of study drug. During that time subjects receive regular meals and snacks are available. Subjects are particularly observed for signs of hypoglycemia.

Blood samples for PK and PD are obtained at times designated in the final protocol. Blood for biomarkers are obtained.

Baseline values are obtained for the following assessments:
Functional—Walking (10 mW/RT, TUG)
Functional—Myotonia (HGRT, VHOT)
Functional—Strength/Fine Motor (QMT, MIRS)
Symptom-Related (DEXA scan, FVC/FEV₁, BPI, FSS, ESS, BDI)
Patient impact activity assessments—(MDHI, DM1-Activ-C)
Patient-reported Outcomes—(CGI-S, PGI-S, INQoL, LEFS)

On Study Day 2, final PK and PD samples are taken. Vital signs are taken and adverse events are assessed. A 12-lead ECG is extracted from the Holter monitor and the Holter monitor is disconnected. Instructions regarding food intake and hypoglycemic symptoms are reinforced. Participants are discharged from the study unit

### On Treatment Study Visits on Day 5 and at Week 3, Week 6, Week 9 and Week 12 (End of Treatment

During these visits, vital signs and adverse events are assessed. Concomitant medications are reviewed. Safety-related laboratory tests (hematology, chemistry, urinalysis and coagulation studies) are obtained. Blood for biomarkers are obtained (Week 6 and Week 12 only). Blood samples for PD are obtained at times designated in the final protocol.

Values are obtained for the following assessments as described in the Schedule of Assessments:
Functional—Walking (10 mW/RT, TUG)
Functional—Myotonia (HGRT, VHOT)
Functional—Strength/Fine Motor (QMT, MIRS)
Symptom-Related (DEXA scan, FVC/FEV₁, BPI, FSS, ESS, BDI)
Patient impact activity assessments—(MDHI, DM1-Activ-C)
Patient-reported Outcomes—(CGI-S, PGI-S, INQoL, LEFS)

Instructions regarding food intake and hypoglycemic symptoms are reinforced.

### Follow-Up Study Visits at Week 15 and Week 21

During these visits, vital signs and adverse events are assessed. Concomitant medications are reviewed. Safety-related laboratory tests (hematology, chemistry, urinalysis and coagulation studies) are obtained. Blood samples for PD are obtained at times designated in the final protocol. Blood for biomarkers is obtained (Week 24 only).

If adverse events are on-going at the EOS visit, they are followed until resolution or stabilization.

### Duration of Follow Up

Following administration of study drug weekly for 12 weeks, subjects are followed at the investigative site for 12 additional weeks until the End of Study Visit on Study Week 24.

### Safety Considerations & Stopping Rules

All subjects are monitored closely by the CRU nursing staff and investigators during the procedures to prevent and minimize any potential risks or discomfort. The nursing staff and study personnel receives in-service training regarding these procedures. Each participant is asked to report any continuing discomfort caused by any of the performed procedures.

### Safety Monitoring Related to Mechanism of Action of HSA-IGF2-R61A

HSA-IGF2-R61A is a recombinant fusion protein (biologic) comprised of human serum albumen (HSA) linked to a human insulin-like growth factor-2 sequence (HSA-IGF2-R61A). Insulin-like Growth Factor-2 (IGF-2) is a mitogenic peptide hormone that is structurally similar to insulin. It binds to IGF-1R and the insulin receptor A (IR-A) on the surface of target cells. Two currently-marketed products incorporating IGF-1 (IPLEX® and INCRELEX®), used for treating growth hormone deficiency, may be associated with hypoglyemia. Doege-Potter syndrome, an extremely rare fibrous tumor that results in excess secretion of IFG-2, is likewise associated with hypoglycemia. Thus, hypoglycemia is a potential side effect of HSA-IGF2-R61A related to its mechanism of action. During the study, careful monitoring of glucose is performed, particularly in the 24 hours following each administration of study drug. The study drug is administered in a fed state to mitigate post-dosing hypoglycemia. Subjects and their families are educated about the need to maintain a regular meal schedule throughout the study. Subjects are educated about symptoms related to hypoglycemia. This education is reinforced by study site staff at each study visit.

Administration of products incorporating IGF-1 to children with growth hormone deficiency has been associated with minor elevations in hepatic enzymes. This study incorporates routine monitoring of hepatic enzymes through the duration of the dosing and follow-up periods.

### Immune Monitoring

Since human protein therapeutics may be associated with immune responses, subjects are monitored for anti-drug antibodies (ADAs) and neutralizing antibodies (Nabs) through validated activity assays during the course of the study. Sampling for immunogenicity occurs in parallel with pharmacokinetic sampling and at timepoints corresponding to predicted peak antibody responses.

### Stopping Rules for Individual Subjects

Subjects may voluntarily withdraw from the study for any reason at any time. Subjects are withdrawn from the study if intolerable side effects or severe adverse events, as determined by the PI, are identified. Subjects who cannot tolerate the medication or procedures, have surgery or other illnesses which affect the evaluations have the drug discontinued.

Specific stopping rules include:
ALT or AST values ≥3×ULN associated with total bilirubin ≥2×ULN, with no underlying medical conditions to explain the elevated values
Uncontrolled hypoglycemia

### Data & Safety Monitoring (DSM)

This study is conducted according to the ICH E6(R3) risk and quality processes described in the applicable procedural documents. Accurate eCRFs and source documentation are maintained. An external contract research organization (CRO) is responsible for activities associated with the data management of this study. This includes setting up a relevant database and data transfer mechanisms, along with appropriate validation of data and resolution of queries. Data generated within this clinical study are handled according to the relevant Standard Operating Procedures (SOPs) of CRO. Investigative site personnel enter subject data into an EDC system. The analysis data sets are a combination of these data and data from other sources (e.g., laboratory data). Clinical data management is performed in accordance with applicable CRO standards and data cleaning procedures to ensure the integrity of the data, e.g., removing errors and inconsistencies in the data. Adverse event terms are coded using MedDRA, an internal validated medical dictionary, and concomitant medications are coded using the World Health Organization Drug Dictionary.

A medical monitor reviews and assesses all safety data as they become available throughout the study.

A study monitor (clinical research associate) from the CRO monitors the study and site activities to verify that data are authentic, accurate, and complete; the safety and rights of subjects are being protected and the study is being conducted in accordance with the currently approved protocol and any other study agreements, GCP and all applicable regulatory requirements. The monitor checks that the CRFs have been correctly completed, with comparison of source documents and check that AEs have been documented. All relevant study-related documents and source data must be available at the study site to document the existence of the research subjects and to substantiate the integrity of study data collected. Source data must include the original documents relating to the study, as well as the medical treatment and medical history of the subject.

A Data Safety Monitoring Board (DSMB), comprised of at least three members: two physicians (e.g., neurologist, internist) with experience in myotonic dystrophy and a statistician otherwise unaffiliated with the study, provide safety oversight for this study. The DSMB operates under a study-specific Charter that is agreed by the Committee members prior to screening the first study subject. The DSMB reviews available pharmacokinetic (PK) data, laboratory data and clinical observations, including adverse events after approximately 3 patients have completed their initial dosing visits, after 12 patients have completed their initial dosing visits, after approximately 12 patients have completed their Month 3 dosing visit and after approximately 24 patients have completed their Month 3 dosing visit.

At the end of each DSMB meeting, the DSMB documents in writing the decisions taken.

### Secondary Studies

PK assessments are conducted in this study. These data enable more precise selection of doses for a subsequent pivotal study.

PD assessments regarding glucose and insulin levels are obtained periodically during the study to monitor possible hypoglycemia. All study drug dosing occur in the fed state to minimize the risk of hypoglycemia.

A sub-study involving muscle biopsies are conducted, involving 12 study participants.

### Assays & Methodologies

All assays being performed in this study are standardized (including blood levels of HSA-IGF2-R61A and anti-drug antibodies). No specialized methodology is needed to be developed or be employed during the study.

### Statistical Analysis

### Analysis Populations

Three analysis populations are defined for this study.
The Safety Population is defined as all subjects who receive at least 1 dose of study drug and have at least 1 post-baseline safety assessment.
The PK Population is defined as all enrolled subjects for whom at least 1 PK parameter of interest can be calculated. In general, on a parameter-by-parameter basis, an individual subject's data may be excluded from analysis if insufficient data are available for that subject to calculate the specific parameter in question.
The Full Analysis Population is defined as all randomized subjects who complete the study without experiencing major protocol deviations or violations.

### Sample Size

The sample size is typical for exploratory Phase 2a studies. The study allows a reasonable determination of safety and efficacy of HSA-IGF2-R61A at two doses compared to placebo when administered over 12 weeks. Since HSA-IGF2-R61A has not previously been administered to patients with DM1, a potential effect size with regard to an assessment (e.g., 10MWT) cannot be estimated precisely.

Recent observations in interventional studies in patients with DM1 support this sample size. Following 3 months of treatment with active drug (n=13) and placebo (n=10), differences between treatment groups in time required to complete 10MWT suggest that approximately 34 subjects would be required to show significance at a 0.05 level with 80% power. 10MWT data from a study evaluating strength training suggest that a similar number of subjects would be required to show significance at a 0.05 level with 80% power following 3 months of treatment.

As designed, the present study is not necessarily fully powered to provide a definitive assessment of HSA-IGF2-R61A in the study population. However, the cohort sizes are sufficient to generate meaningful comparative change data enabling calculations for powering a subsequent definitive study.

PK has been assessed in healthy normal volunteers (HSA-IGF2-R61A-101). Data obtained in this study allows an adequate definition of the PK profile of HSA-IGF2-R61A in this patient population.

### Safety Analysis

The full detail of the safety analyses is outlined in a statistical analysis plan (SAP). The safety analyses are assessed based on the evaluation of treatment-emergent AE (TEAE) for the entire study. All AEs are coded using the Medical Dictionary for Regulatory Activities (MedDRA) version 27.1 or later. Safety data, including AEs, clinical laboratory test results, 12-lead ECGs and Holter monitors, assessments of immunogenicity and vital sign measurements, are summarized by treatment. Physical examination findings and concomitant medications are listed.

### Pharmacokinetic Analysis

The full detail of the PK analyses is outlined in a statistical analysis plan (SAP). Briefly, the PK analyses is performed using the PK population. Plasma concentrations of HSA-IGF2-R61A are summarized by dose and time point using descriptive statistics. Mean and individual plasma drug concentrations over time are presented in figures using linear and semi log scales. The PK parameters listed above under “Endpoints” are calculated using a non-compartmental analysis and summarized by dose. AUC and Cmax are tested across dose levels for dose proportionality.

### Exploratory Pharmacodynamic Analyses

Change from baseline and absolute values for PD markers for the relevant pathways (TBD) are summarized by dose. Exploratory exposure response analyses for PD markers are performed starting with simple scatterplots (actual values and change from baseline) vs plasma drug concentration; further analyses including modeling of the response may be performed as indicated.

### Long-Term Follow Up

Subsequent to participation in this study (HSA-IGF2-R61A-201), subjects are offered the opportunity to participate in an open-label follow-on study (HSA-IGF2-R61A-201-OLE). At present, it is anticipated that subjects receiving the LO dose of HSA-IGF2-R61A in the present study continue on that dose in the OLE (assuming evidence of efficacy has been demonstrated and no safety issues have been identified). Similarly, it is anticipated that subjects receiving the HI dose of HSA-IGF2-R61A in the present study continue on that dose in the OLE (assuming evidence of efficacy has been demonstrated and no safety issued have been identified). Subjects receiving placebo in the present study who agree to participate in the OLE are randomized evenly to the LO and HI doses of HSA-IGF2-R61A. It is anticipated that the OLE study would last for approximately 12 months. The protocol for this study is developed after approximately 50% of subjects in HSA-IGF2-R61A-201 have completed 3 months of dosing. Schema for study periods is listed in Table 26 and SoA listed in Table 27.

TABLE 26
Schema

| | | Study Periods | | |
|---|---|---|---|---|
| | | Screening | Treatment | Follow-up |
| | N= | (~28 days) | (12 weeks) | (12 weeks |
| HSA-IGF2-R61A-HI SC Q | 20 | [symbol] | | |
| week × 12 | | | | |
| HSA-IGF2-R61A-LO SC Q | 20 | [symbol] | | |
| week × 12 | | | | |

| | | |
|---|---|---|
| Placebo SC Q week × 12 | 20 | [symbol] |

Approximately 60 subjects are randomized (1:1:1) into 3 study cohorts across 6-8 clinical study center:
HSA-IGF2-R61A-LO (n=20)
HSA-IGF2-R61A-HI (n=20)
Placebo (n=20

TABLE 27
Schedule of Activities (SoA)

| | Screen | | Treatment Period | | | | | | | | | | | | | | Follow-up | | | | | | |
|---|---|---|---|---|---|---|---|---|---|---|---|---|---|---|---|---|---|---|---|---|---|---|---|
| | Study Visit | | | | | | | | | | | | | | | | | | | | | | |
| | 1 | | 2 | | 3 | 4 | 5 | 6 | 7 | 8 | 9 | 10 | 11 | 12 | 13 | 14 | 15 | 16 | 17 | 18 | 19 | | |
| | Study Week | | | | | | | | | | | | | | | | | | | | | | |
| | | | | | D5 | W2 | W3 | W4 | W5 | W6 | W7 | W8 | W9 | W10 | W11 | W12 | W13 | W15 | W18 | W21 | W24 | | |
| | D-28 | D-1 | D1 | D2 | ±1d | ±1d | ±2d | ±2d | ±2d | ±2d | ±2d | ±2d | ±2d | ±2d | ±2d | ±2d | ±2d | ±2d | ±3d | ±2d | ±3d | | |
| | Visit Location | | | | | | | | | | | | | | | | | | | | | | |
| | OP | SU | SU | SU | OV | OV | OV | OV | TC | OV | TC | TC | OV | TC | TC | EoT | OV | TC | OV | TC | EoS | ET | |
| Assessment | Time | | | | | | | | | | | | | | | | | | | | | | |
| Informed | X | | | | | | | | | | | | | | | | | | | | | | |
| consent a | | | | | | | | | | | | | | | | | | | | | | | |
| Demography | X | | | | | | | | | | | | | | | | | | | | | | |
| Medical/ | X | | | | | | | | | | | | | | | | | | | | | | |
| surgical history | | | | | | | | | | | | | | | | | | | | | | | |
| Inclusion/ | X | X | | | | | | | | | | | | | | | | | | | | | |
| exclusion criteria b | | | | | | | | | | | | | | | | | | | | | | | |
| Height and | X | X | | | | | X | | | X | | | X | | | X | | | | | X | X | |
| weight c | | | | | | | | | | | | | | | | | | | | | | | |
| Physical | X | X | | | | | | | | X | | | | | | X | | | | | X | X | |
| examination d | | | | | | | | | | | | | | | | | | | | | | | |
| Vital signs e | X | X | X | X | X | X | X | X | | X | | | X | | | X | X | X | | X | X | X | |
| DOA screen f | X | X | | | | | | | | | | | | | | | | | | | | | |
| Pregnancy | X | X | | | | | | | | | | | | | | X | | | | | X | X | |
| testing g | | | | | | | | | | | | | | | | | | | | | | | |
| Serology (HIV, | X | | | | | | | | | | | | | | | | | | | | | | |
| Hep B/C) h | | | | | | | | | | | | | | | | | | | | | | | |
| Randomization i | | X | | | | | | | | | | | | | | | | | | | | | |
| HSA-IGF2-R61A | | | X | | | X | X | X | X | X | X | X | X | X | X | X | | | | | | | |
| or placebo | | | | | | | | | | | | | | | | | | | | | | | |
| Adverse Events j | | X | X | X | X | X | X | X | X | X | X | X | X | X | X | X | X | X | X | X | X | X | |
| Telephone | | | | | | | | | X | | X | X | | X | X | | | X | | X | | | |
| Safety Call k | | | | | | | | | | | | | | | | | | | | | | | |
| Concomitant | X | X | X | X | X | X | X | X | X | X | X | X | X | X | X | X | X | X | X | X | X | X | |
| Medications | | | | | | | | | | | | | | | | | | | | | | | |
| Laboratory | X | X | X | X | X | X | X | X | | X | | | X | | | X | X | | X | | X | X | |
| Studies (see list) l | | | | | | | | | | | | | | | | | | | | | | | |
| Covid testing l | | X | | | | | | | | | | | | | | | | | | | | | |
| | | Screen | | Treatment Period | | | | | | | | | | | | | | Follow-up | | | | | |
| | | Study Visit | | | | | | | | | | | | | | | | | | | | | |
| | | 1 | | 2 | | 3 | 4 | 5 | 6 | 7 | 8 | 9 | 10 | 11 | 12 | 13 | 14 | 15 | 16 | 17 | 18 | 19 | |
| | | Study Week | | | | | | | | | | | | | | | | | | | | | |
| | | | | | | D5 | W2 | W3 | W4 | W5 | W6 | W7 | W8 | W9 | W10 | W11 | W12 | W13 | W15 | W18 | W21 | W24 | |
| | | D-28 | D-1 | D1 | D2 | ±1d | ±1d | ±2d | ±2d | ±2d | ±2d | ±2d | ±2d | ±2d | ±2d | ±2d | ±2d | ±2d | ±2d | ±3d | ±2d | ±3d | |
| | | Visit Location | | | | | | | | | | | | | | | | | | | | | |
| | TIME | OP | SU | SU | SU | OV | OV | OV | OV | TC | OV | TC | TC | OV | TC | TC | EoT | OV | TC | OV | TC | EoS | ET |
| Assessment | | | | | | | | | | | | | | | | | | | | | | | |
| Muscle Biopsy (sub- | | | X | | | | | | | | | | | | | | | X | | | | | |
| study) r | | | | | | | | | | | | | | | | | | | | | | | |
| CSSRS | X | X | | | | | | | | | X | | | | | | X | | | | | X | X |
| BIOMARKER s | | | | | | | | | | | | | | | | | | | | | | | |
| Biomarker-Target | | | | X | | | | X | | | X | | | X | | | X | | | | | X | X |
| Biomarker- | | | | X | | | | X | | | X | | | X | | | X | | | | | X | X |
| Muscle | | | | | | | | | | | | | | | | | | | | | | | |
| Biomarker- | | | | X | | | | X | | | X | | | X | | | X | | | | | X | X |
| Metabolism | | | | | | | | | | | | | | | | | | | | | | | |
| WALKING | | | | | | | | | | | | | | | | | | | | | | | |
| 10-m walk/run | 10 | X | X | | | | | | | | X | | | | | | X | | | | | X | X |
| time | min | | | | | | | | | | | | | | | | | | | | | | |
| Timed Up and Go | | X | X | | | | | | | | X | | | | | | X | | | | | X | X |
| (TUG) | | | | | | | | | | | | | | | | | | | | | | | |
| MYOTONIA | | | | | | | | | | | | | | | | | | | | | | | |
| Hand Grip | 5 | | X | | | | | | | | X | | | | | | X | | | | | X | X |
| relaxation time | min | | | | | | | | | | | | | | | | | | | | | | |
| Video hand | 5 | | X | | | | | | | | X | | | | | | X | | | | | X | X |
| opening time | min | | | | | | | | | | | | | | | | | | | | | | |
| STRENGTH/FINE | | | | | | | | | | | | | | | | | | | | | | | |
| MOTOR | | | | | | | | | | | | | | | | | | | | | | | |
| QMT | 10 | | X | | | | | | | | X | | | | | | X | | | | | X | X |
| | min | | | | | | | | | | | | | | | | | | | | | | |
| MIRS | 2 | | X | | | | | | | | X | | | | | | X | | | | | X | X |
| | min | | | | | | | | | | | | | | | | | | | | | | |
| Grip strength | 5 | | X | | | | | | | | X | | | | | | X | | | | | X | X |
| | min | | | | | | | | | | | | | | | | | | | | | | |
| SYMPTOM- | | | | | | | | | | | | | | | | | | | | | | | |
| RELATED | | | | | | | | | | | | | | | | | | | | | | | |
| Brief Pain | 10 | | X | X | | | | | | | X | | | | | | X | | | | | X | X |
| Inventory | min | | | | | | | | | | | | | | | | | | | | | | |
| Fatigue Severity | 5 | | X | X | | | | | | | X | | | | | | X | | | | | X | X |
| Scale | min | | | | | | | | | | | | | | | | | | | | | | |
| Epworth | 5 | | X | X | | | | | | | X | | | | | | X | | | | | X | X |
| Sleepiness Scale | min | | | | | | | | | | | | | | | | | | | | | | |
| Beck Depression | 10 | | X | X | | | | | | | X | | | | | | X | | | | | X | X |
| Inventory | min | | | | | | | | | | | | | | | | | | | | | | |
| ACTIVITY/ | | | | | | | | | | | | | | | | | | | | | | | |
| IMPACT | | | | | | | | | | | | | | | | | | | | | | | |
| DM1-Activ-C | 5 | | X | | | | | | | | X | | | | | | X | | | | | X | X |
| | min | | | | | | | | | | | | | | | | | | | | | | |
| MDHI | 20 | | X | | | | | | | | X | | | | | | X | | | | | X | X |
| | min | | | | | | | | | | | | | | | | | | | | | | |
| PATIENT- | | | | | | | | | | | | | | | | | | | | | | | |
| REPORTED | | | | | | | | | | | | | | | | | | | | | | | |
| OUTCOMES | | | | | | | | | | | | | | | | | | | | | | | |
| CGI-Improvement | 3 | | | | | | | X | | | X | | | | | | X | | | | | X | X |
| (clinician) | min | | | | | | | | | | | | | | | | | | | | | | |
| CGI-Severity | 3 | | | X | | | | | | | | | | | | | | | | | | | |
| (clinician) | min | | | | | | | | | | | | | | | | | | | | | | |
| CGI-Severity | 3 | | | X | | | | | | | | | | | | | | | | | | | |
| (patient/caregiver) | min | | | | | | | | | | | | | | | | | | | | | | |
| CGI- | 3 | | | | | | | X | | | X | | | | | | X | | | | | X | X |
| Improvement | min | | | | | | | | | | | | | | | | | | | | | | |
| (pt/caregiver) | | | | | | | | | | | | | | | | | | | | | | | |
| InQoL | 20 | | | X | | | | | | | X | | | | | | X | | | | | X | X |
| | min | | | | | | | | | | | | | | | | | | | | | | |
| Lower extremity | 15 | | | | | | | | | | | | | | | | | | | | | | |
| functional scale | min | | | | | | | | | | | | | | | | | | | | | | |

Abbreviations: OP = out-patient; SU = study unit; OV = out-patient visit; TC = telephone call; CSSRS = Columbia-Suicide Severity Rating Scale; DOA = Drugs of abuse; FSH = Follicle stimulating hormone; EoT = End of Treatment; EoS = End of Study; ET = Early Termination; QMT = quantitative muscle testing; MIRS = Muscular impairment rating scale; MDHI = myotonic dystrophy health index; TIME = approximate time to complete procedure; CGI-S = clinical global impression-Severity; CGI-I = clinical global impression-improvement; InQoL = Individualized neuromuscular quality of life

a. Informed consent may be obtained prior to Study Visit 1
All inclusion/exclusion criteria should be assessed during screening and confirmed prior to randomization (Study Day −1).
c. Height and weight are measured with shoes off during Screening. BMI is calculated during Screening. Weight only (with shoes off) is collected at Study Day −1 and at the Week 12 Study Visit (EOT) and at Early Termination (ET). Waist and hip circumference measurements will be obtained during Screening and during study visits at Week 6 and Week 12.
d. A full physical examination will be performed during Screening and at the Week 12 Study Visit (EOT) and at Early Termination (ET). Unscheduled symptom-directed physical examinations may be conducted at any time at the Investigator's discretion.
e. Vital signs include the following assessments: blood pressure, pulse rate, respiratory rate and body temperature. Vital signs will be performed after the participant has been resting for at least 5 minutes. Body temperature will be assessed during Screening, after admission to the Study Unit on Study Day −1, prior to dosing with study drug on Study Day 1 and prior to collection of blood samples at the Week 12 Study Visit (EOT) and at Early Termination. On all other occasions, blood pressure, pulse rate (heart rate) and respiratory rate will be obtained prior to collection of PK samples and 10 minutes after completion of PK sampling. At each designated timepoint, the assessment window is ±20 minutes.
f. Drug testing is for the following substances: amphetamines, barbiturates, benzodiazepines, cocaine, MDMA, methamphetamines, methadone, opiates, oxycodone, PCP, tricyclic antidepressants, and THC
g. Any female subject who does not meet the criteria for non-childbearing is considered to be of childbearing potential. For any suspected postmenopausal female (greater than 50 years of age with spontaneous amenorrhea for 12 months or longer), FSH will be measured (Serum FSH levels must be ≥40 mIU/mL for the participant to be considered postmenopausal). For all female subjects of childbearing potential, pregnancy tests will be performed. A serum pregnancy test will be performed during Screening. A urine β-hCG test will be performed on Study Day −1 and at the Week 12 Study Visit (EOT) (and at Early Termination). If the urine β-hCG test is reported as positive, the result will be confirmed with a serum pregnancy test.
h. Serology for HIV (antigen/antibody test), HBV (HBsAg) and HCV (HepC Ab)
i. After eligibility has been confirmed, the patient will be randomized to one of three treatments (HSA-IGF2-R61A-HI, HSA-IGF2-R61A-LO or placebo) according to the randomization plan developed by the study statistician.
j. Adverse events will be monitored for individual subjects from the time of signing of the informed consent through their discharge from the study.
k. Telephone check-ins will be conducted during the Treatment Period during Study Weeks 5, 7, 10 and 11 and during the Follow-up period during Study Week 15 and Week 21 to review concomitant medications and inquire about AEs.
l. Hematology, serum chemistry, coagulation assessments and urinalyses will be collected at various points during the study as noted above. Assessments are detailed in the accompanying table. Covid testing will performed during Screening and on Study Day −1; testing may be repeated at any time during the study at the Investigator's discretion. Laboratory samples will be obtained with PK samples on Study Day 5 and at visits on Week 2, Week 3, Week 6, Week 9 and at the Week 12 Study Visit (EOT) and at Early Termination. On Study Day 1, blood samples for laboratory assessments will be drawn in the fasted state prior to administration of study drug. eGFR will only be calculated during Screening using The CKD-EPI Creatinine-Cystatin C Age, Sex Equation (2021) [see Laboratory Tests and Assessments Description, below]. An oral Glucose Tolerance Test (OGTT) will be performed on Study Day −1 and at study visits at Week 2 and Week 9 during the Treatment Period and at the Week 21 visit during the follow-up period. At these times, samples will also be drawn for insulin, hemoglobin A1c and C peptide and insulin resistance will be calculated. Lipid profiles will be drawn at Screening, on Study Day −1 and during study visits at Week 2, Week 3, Week 6 and at the Week 12 Study Visit (EOT). During the Follow-up Period, lipid profiles will be obtained at the Week 13, Week 21 and Week 24 (EOS) study visits.
m. Standard (safety) digital ECGs will be performed during Screening and on Study Day −1 and at visits at Week 3, Week 6 and at the Week 12 Study Visit (EOT) and at Early Termination. All standard (safety) digital ECGs will be performed in triplicate (approximately 1 minute apart) after the subject has been supine for ≥5 minutes. Additional 12-lead ECGs may be performed at any other time if clinically indicated. A hard copy of the ECG will be printed and signed by the investigator at the investigative site. Abnormal findings will be assessed by the investigator as either clinically significant (CS) or not clinically significant (NCS).
n. 24-hour Holter monitoring will be initiated prior to administration of study drug on Study Day 1. Digital ECGs will be extracted at times of PK sampling. At each time, a printed copy will be reviewed at the study site while an electronic copy will be transmitted to the central reading facility for review and formal analysis.
o. Spirometry will be obtained during the Screening Period and at the Week 12 Study Visit (EOT).
p. A 3-D transthoracic echocardiogram will be obtained during the Screening Period (to ensure the subject meets enrollment criteria) and at the Week 12 Study Visit (EOT)
q. PK sampling (plasma) will be collected pre-dose and at 0.5, 1, 1.5, 2, 3, 4, 6, 8, 12, 16 and 24 hours (±10 minutes at each time point) after the start of administration of the respective study drugs. Subjects will then be discharged from the Study Unit. Subjects will return for outpatient visits on Study Day 5 and at visits at Week 2, Week 3, Week 6 at the Week 12 Study Visit (EOT); at these visits, samples for PK will be drawn prior to administration of study drug (trough levels). If a subject terminates the study prior to completion of the Week 12 Study Visit, the investigator is encouraged to obtain a PK sample prior to termination if possible.
r. Blood samples for immunogenicity testing will be drawn on Study Day 1 (pre-dose, with PK sample) and with PK samples during Study Visits at Week 3, Week 6, Week 9 and Week 12 (EOT) during the Treatment Period and Week 24 (EOS) during the Follow-up Period.
s. Blood samples for biomarkers (see Assessment Description, below) will be obtained per the schedule of events. Processing for retention and analysis will be described in the Laboratory Manual for the study.
   NOTE: When multiple study procedures are scheduled at the same time point, the order of procedures should be as follows: perform ECG, obtain heart rate and blood pressure, and collect PK samples. At times when study procedures are scheduled simultaneously, PK blood samples must be collected within the designated windows at the scheduled time points; other procedures will be performed as close to the scheduled time point as possible.

### Laboratory Tests

Clinical laboratory evaluations are performed centrally. Decisions regarding eligibility for this study may be made using local laboratory determinations. For dosing decisions, local hematology and chemistry laboratory results may be used. Handling and shipment of clinical laboratory samples are outlined in the study manual.

Clinical laboratory evaluations are performed as outlined below. Blood samples for analysis of the following clinical chemistry and hematological parameters are obtained as specified in the Schedule of Events. Table 28 lists Laboratory tests performed. Table 29 lists descriptions of Assessments and Procedures.

TABLE 28
Laboratory Tests

| | | |
|---|---|---|
| Hematology | Hemoglobin | MCH |
| | Hematocrit | MCHC |
| | RBC count | MCV |
| | Platelet (count) | WBC count with differential (absolute & %) |
| Serum Chemistry | Sodium | Total and indirect bilirubin |
| | Potassium | Alkaline phosphatase |
| | Chloride | ALT |
| | Bicarbonate | AST |
| | Glucose | Lactate dehydrogenase (LDH) |
| | Calcium | Gamma glutamyl transferase (GGT) |
| | Creatinine | Inorganic phosphate |
| | Blood urea nitrogen | Magnesium |
| | (BUN) | Creatine phosphokinase (CPK) |
| | Total protein | |
| | Albumin | |
| Glucose | Hemoglobin A1C | Insulin |
| metabolism & | C-peptide | Insulin resistance (HOMA-IR, QUICKI) |
| insulin sensitivity | | |
| Coagulation | PT | INR |
| | PTT | |
| Urinalysis | Specific gravity | Urine blood |
| | pH | Urine ketones |
| | Leukocyte esterase | Urine bilirubin |
| | Urine protein | Urine glucose |
| Lipid levels | Total cholesterol | HDL-C |
| | LDL-C | triglycerides |
| Screening | HIV (antigen/antibody) | HCV (HepC Ab) |
| Serology | HBV (HBsAg) | |
| Immunogenicity | ADA antibodies | Neutralizing antibodies |
| Other | Cystatin C | eGFR: CKD-EPI Creatinine-Cystatin C Age, Sex |
| | | Equation (2021) [See below] |
| Cardiovascular | Echocardiogram | Safety ECG |
| | | Holter Monitoring with ECG extractions |

### CKD-EPI Creatinine-Cystatin C Age, Sex Equation (2021)

$$eGFR = 135 \times {min ( Scr / k , 1 )}^{α} \times {max ( Scr / k , 1 )}^{- 0.544} \times {min ( S cys / 0.8 , 1 )}^{- 0.323} \times {max ( S cys / 0.8 , 1 )}^{- 0.778} \times {0.9961}^{age} \times 0.963 [ if female ]$$

where
Scr=serum creatinine in mg/dL,
Scys is serum cystatin C in mg/dL,
k is 0.7 for females and 0.9 males,
α is −0.219 for females and −0.144 for males,
min indicates the minimum of Scr/k or 1,
   max indicates the maximum of Scr/k or 1]

TABLE 29
Assessments and Procedures - Description

| Assessment | Description |
|---|---|
| Waist and hip | For waist circumference measurement, the participant stands with arms at the sides, feet |
| circumference | together, and abdomen relaxed. Using a Gulick tape measure, a horizontal measure is |
| measures | taken at the narrowest part of the torso (above the umbilicus and below the xiphoid |
| | process). For hip circumference measurement, the participant stands erect and feet |
| | together, a horizontal measure is taken at the maximal circumference of buttocks. |
| | The Gulick tape measure (provided by the Sponsor) is a non-stretch, non-metallic tape |
| | measure that provides a known amount of tension while a measurement is being taken. |
| Glucose Tolerance | A 2-hour oral glucose tolerance test (GTT) is performed under fasted conditions. |
| Test (Glucose and | Participants are administered 75 grams (g) glucose solution prior to administration of the |
| insulin levels) | first dose of study drug and on Study Day 15. Glucose and insulin levels are obtained at |
| | each time point. |
| Lipid Profiles | Total Cholesterol Level; Low-density Lipoproteins (LDL-C) Level; High-density |
| | Lipoproteins (HDL-C) Level; Triglycerides Level |
| Insulin Sensitivity | QUICKI |
| | The QUICKI is based on fasting glucose and insulin measurements and are calculated |
| | using the following equation: |
| | QUICKI = 1/[log(fasting glucose in mg/dL) + log (fasting insulin in uU/mL)] |
| | ISI-MATSUDA |
| | The ISI-Matsuda is based on the average glucose and insulin values obtained during the |
| | entire oral glucose tolerance test and are calculated using the following equation: |
| | ISI = 10,000/√[fasting glucose (mg/dL) × fasting insulin(uU/mL) × mean glucose × |
| | mean insulin] |
| DEXA Scan | A DEXA scan is a non-invasive procedure requiring the participant to only lay still on a |
| (Dual-energy X- | padded table. It is a fast procedure that provides a variety of body composition variables |
| ray | (bone mass/area/density, fat mass, and fat free mass). The DEXA scan exposes individuals |
| absorptiometry) | to low levels of radiation, approximately 0.18 millirem. DEXA measures are described as |
| | having no observable or biological effect and are similar to natural background levels of |
| | radiation in most countries. The participants are asked to notify the technician if they think |
| | they may be pregnant before having the DEXA scan. |
| | Several values are determined, including lean body mass, free fat mass, total body muscle |
| | mass, total body fat percentage |
| 12-lead ECG & | A 12-lead, 24-hour Holter recorder is used to collect safety ECGs, at times designated in |
| Holter monitor | the Schedule of Assessments, and to provide 24-hour arrhythmia monitoring. Safety |
| | ECGs are “captured” at the appropriate time. They are transmitted to a central reading |
| | facility. Copies are printed for local review. |
| | A Holter monitor recording is obtained during the Screening period to ensure that |
| | participants meet enrollment criteria. |
| Spirometry | Sitting comfortably upright, in ambient conditions, participants perform a series of 3 |
| | strong inhalations, followed by a fast a strong exhalation. Forced vital capacity and |
| | forced expiratory volume are measured (in liters). |
| Echocardiogram | An echocardiogram is an ultrasound test that checks the structure and function of the |
| | participant's heart. A transthoracic, 3-dimensional echocardiogram is performed on 2 |
| | occasions during the study. During the Screening Period, an echocardiogram is |
| | performed to ensure the subject meets enrollment criteria. The second is performed at the |
| | end of the dosing period (Week 12). |
| Pharmacokinetics | PK determinations include at least Cₘₐₓ, Tₘₐₓ, estimated terminal phase half-life in plasma |
| | (t_{1/2λz}); area under the concentration-time curve (AUC) from time 0 to 24 h after |
| | administration (AUC_{0-24 h}) and percent dose excreted in urine. |
| Muscle biopsy | Approximately 4 subjects randomized to HSA-IGF2-R61A-LO dose, 4 subjects |
| (sub-study) | randomized to HSA-IGF2-R61A-HI dose and 4 subjects randomized to placebo, following |
| | informed consent, undergo a muscle biopsy on 2 occasions during the study. |
| | A 1 cm incision is made in skin previously disinfected and anesthetized at 15 cm above the |
| | patella to access the vastus lateralis muscle. Suction-modified Bergström muscle biopsy |
| | technique is performed to obtain the muscle sample. The incision is closed by sutures and |
| | participants are instructed about proper post-biopsy care. |
| | Muscles samples are immediately rinsed in a cold phosphate buffered saline solution |
| | (PBS) and frozen in tissue freezing medium in isopentane cooled in liquid nitrogen. The |
| | samples are then stored at −80° C. until further use. |
| Columbia-Suicide | The Columbia Protocol, also known as the Columbia-Suicide Severity Rating Scale (C- |
| Severity Rating | SSRS), supports suicide risk assessment through a series of simple, plain-language |
| Scale (C-SSRS) | questions that anyone can ask. The answers help users identify whether someone is at risk |
| | for suicide, assess the severity and immediacy of that risk, and gauge the level of support |
| | that the person needs. |
| Biomarker-Target | IGF-1. Other markers may be identified and included in testing. |
| Biomarker-Muscle | CK, troponin, follistatin, myostatin; Titin-N fragment (UA) (titin/Cr); Neurofilament light |
| | protein (NfL); DAPK1; Calpain-3 (CAN3); Cysteine and glycine-rich protein 3 (CSPR3). |
| | Additional targets may also be assessed. |
| Biomarker- | Glucose, insulin, insulin resistance, GTT, hemoglobin A1C, lipids, glucagon, c peptide |
| Metabolism | |
| Timed Up and Go | TUG is used to assess a person's mobility and requires both static and dynamic balance. |
| test (TUG) | During the test, the participant rises from a chair, walks 3 meters, turns around 180 |
| | degrees, walks back to the chair and sits down in the chair while turning 180 degrees. |
| | Time (seconds) is recorded from “Go!” until the subject is completely seated in the chair. |
| | During the test, the participant is expected to wear their regular footwear. In this study, no |
| | mobility aids are permitted. |
| | The test is performed twice at each administration: at Screening (for familiarization) and |
| | on Day −1 (baseline value), at Week 6, Week 12 (End of Treatment, EOT) and Week 24 |
| | (End of Follow-up). A 60-second rest interval is provided between each pair of tests. The |
| | test is be performed at Early Termination Visits (if possible). |
| | The test is scored using a stopwatch. Times are recorded for each attempt. Values for each |
| | visit are recorded and averaged. The average value is used for further assessments. |
| 10-meter | These tests are used to measure walking speed (muscle endurance). Tests are conducted at |
| Walk/Run Test | 2 different paces: comfortable walking and maximal pace (running is not permitted with |
| (10MW/RT) | either pace). A “track” is laid out on a flat surface. One mark is set at 0 meters, another at |
| | 2 meters, another at 12 meters and the last at 14 meters. The total distance the subject |
| | walks is 14 meters. |
| | Participants start on the 0-meter mark. They are asked to walk at a comfortable speed. As |
| | the subject hits the 2-meter mark, timing is started. It is stopped as the participant passes |
| | the 12-meter mark. The participant decelerates and stops at the 14-meter mark. After a 10- |
| | minute wait, the same procedure is followed for the 10-meter walk test at maximum speed. |
| | Walking time is measured to the nearest 0.001 sec using a digital stopwatch as the lead foot |
| | crosses markers at 2 and 12 meters, respectively. The time is rounded to the closest |
| | 0.01sec. Gait speed is calculated as meters per second (m/s). |
| | Testing is administered at Screening to provide initial experience with the procedure and |
| | at the following study visits: Day −1 (baseline), at Week 6, Week 12 (End of Treatment, |
| | EOT) and Week 24 (End of Follow-up). |
| Video hand | The dominant wrist and hand are placed on a bedside table with the forearm fully |
| opening time | supinated. The participant is asked to open the hand after making a tight fist for 3-5 |
| (vHOT) | seconds. A digital video recording is made of the hand opening. The video field of view |
| | only includes the participant's forearm and hand. The time required for hand opening. |
| | which often ranges from 3 to 20 seconds in individuals with DM1, is determined by |
| | blinded review of the video recording. The procedure is carried out three times over |
| | approximately 30 minutes. |
| Quantitative | Maximum Voluntary Isometric Contraction Testing (MVICT) of the limb muscles is |
| Myometry (QMT) | performed using an adjustable cuff to attach the patient's arm or leg to an inelastic strap |
| | that is connected to a force transducer with a load of 0.5 to 1,000 Newtons. Each muscle |
| | is tested twice while the patient is encouraged by the evaluator to exert maximal effort. |
| | The maximum force generated by the patient is recorded for each trial and the maximum |
| | over the two trials is used as the final measurement for each muscle. Seven muscle |
| | groups are tested bilaterally (shoulder abductors, biceps, triceps, quadriceps, hamstrings, |
| | ankle flexors and hand grip) for a total of twelve muscle groups. |
| Muscular | The MIRS score is a disease-specific rating scale, based on manual muscle testing that |
| Impairment Rating | assesses disease status (and progression) as measured by muscle weakness through |
| Scale (MIRS) | manual muscle testing. Eleven muscle groups are assessed (10 are bilateral) and scored on |
| score | a 5-point Modified Medical Research Council Scale (modifications enable subdivisions at |
| | 3.5 and 4.5). The total possible muscle score is 55 points for each side. Both sides are |
| | scored. |
| Maximal | Hand grip strength is measured using an isometric dynamometer (JAMAR ®, Sammons; |
| voluntary hand | provided by sponsor). The grip width is adjusted to hand size and with arm flexed at 90°. |
| grip assessment | The participant performs three 5 second efforts with a one-minute rest between trials. |
| Brief Pain | The BPI is a self-report measure that assesses the intensity of pain, impact of pain on daily |
| Inventory (BPI) | function, location of pain, pain medications, and amount of pain relief in the prior 24 hours |
| | or the prior week. The BPI allows patients to rate the severity of their pain and the degree |
| | to which their pain interferes with common dimensions of feeling and function. |
| | The BPI starts with an open question about the presence of pain and includes a body chart |
| | to indicate areas of pain and the part of the body with the worst pain, with response |
| | alternatives for describing the characteristics of the pain. |
| | This is followed by two categories of questions assessing, respectively: Pain Intensity (4- |
| | item measures of pain severity) and Pain Interference (7-item assessment of the impact of |
| | pain of 7 aspects of function). Each item is rated on an 11-point scale (0 to 10). |
| Fatigue Severity | The FSS is a 9-item questionnaire that assesses the severity of fatigue and its impact on |
| Scale (FSS) | daily activities and lifestyle. Participants rate how fatigue interferes with specific |
| | activities. |
| | Responses are scored on a 7-point scale, where 1 represents “strongly disagree” and 7 |
| | represents “strongly agree.” The total score ranges from 9 to 63, with higher scores |
| | indicating greater fatigue severity. |
| Epworth | The ESS is an eight-item questionnaire regarding the likelihood that the participant doze |
| Sleepiness Scale | or fall asleep (in contrast to “being tired”) in various situations. |
| (ESS) | Responses are scored on a 4-point scale where “0” represents “never doze or sleep” and 3 |
| | represents “high chance of dozing or sleeping.” The total score ranges from 0 to 24 with |
| | higher scores indicating a greater likelihood of dozing or sleeping; 0-10 is a normal range |
| | for healthy adults. |
| Beck Depression | The BDI is a self-report questionnaire used to measure the severity of depression. The BDI |
| Inventory (BDI-II) | includes 21 items, each of which corresponded to a symptom of depression. The |
| | instrument was revised following changes in the Diagnostic and Statistical Manual of |
| | Mental Disorders (DSM). The revised BDI is known as the BDI-II. The 21 items are |
| | grouped into four categories: Somatic (physical) symptoms, affective (emotional) |
| | symptoms, cognitive symptoms and vegetative symptoms (sleep patterns and appetite). |
| | Responses are scored on a 4-point scale where 0 represents no changes or worries and 3 |
| | represents the most serious assessment of the respective item. The total score ranges from |
| | 0 to 63 with higher scores suggesting greater levels of depression. 0-10 is a normal range |
| | for healthy adults. |
| DM1-ACTIV^{C} | Patients complete the questionnaire after having received written and verbal instructions. |
| | The patient should be comfortably seated in a quiet room. |
| | The patient is to provide answers assessing the impact of DM1 rather than another |
| | concomitant disease (e.g., arthritis). |
| | The patient is encouraged to complete the questions by themselves. However, a partner, |
| | relative or friend may be of assistance if the patient is unsure or has communication |
| | problems. |
| Myotonic | The MIDHI is a disease-specific, patient-reported outcome measure to estimate the overall |
| Dystrophy Health | disease burden and the impact of key symptomatic themes in DMI patients. Subscales of |
| Index (MDHI) | the MIDHI measure a patient's perception of their health as it relates to: 1) Mobility; 2) |
| | Upper Extremity Function; 3) Ability to do Activities; 4) Fatigue; 5) Pain; 6) |
| | Gastrointestinal Issues; 7) Vision; 8) Communication; 9) Sleep; 10) Emotional Issues; 11) |
| | Cognitive Impairment; 12) Social Satisfaction; 13) Social Performance; 14) Myotonia; |
| | 15) Breathing; 16) Swallowing; and 17) Hearing. |
| CGI-S/PGI-S | The CGI-S (clinician) is a single-item question with a 7-point rating system designed to |
| | provide a brief, stand-alone assessment of the clinician's view of the severity of the |
| | subject's myotonic dystrophy, compared to other patients the clinician has seen. |
| | The CGI-S (patient/caregiver) is a single-item question with a 7-point rating system |
| | designed to assess the participant's (or caregiver's) assessment of the severity of the |
| | participant's myotonic dystrophy. |
| | These ratings are assessed prior to beginning treatment with study drug. |
| CGI-I (Clinician)/ | The CGI-I (clinician) is a single-item question with a 7-point rating system (responses |
| CGI-I | ranging from “much improved” to “very much worse”) designed to provide a brief, stand- |
| (Patient/Caregiver) | alone assessment of the clinician's view of the subject's global functioning after initiating |
| | study drug, compared to their global functioning just prior to initiating treatment. |
| | The CGI-I (patient/caregiver) is a single-item question with a 7-point rating system |
| | (responses ranging from “much improved” to “very much worse”) designed to provide a |
| | brief, stand-alone assessment of the patient's/caregiver's view of the patient's global |
| | functioning after initiating treatment. |
| | These ratings are assessed after treatment has started. |
| Individualized | The INQoL is a measure of health-related quality of life and is designed specifically for |
| neuromuscular | people with neuromuscular disease. The scale is used for people to rate the severity of |
| quality of life | symptoms and functional problems that they experience. |
| (InQoL) | The INQoL questionnaire comprises 45 questions on 7 or 8-point scales, grouped in |
| | categories covering aspects of muscle weakness, muscle locking, pain, tiredness (fatigue), |
| | activities, independence, relationships, “how you feel” (anxiety, depression, frustration, |
| | self-esteem), “the way you look”, and aspects of treatment. Subsequent questions then |
| | explore the extent to which these issues affect or limit a patient's day to day life. |
| | The INQoL is the only validated HRQoL questionnaire that refers specifically to the |
| | presence and impact of myotonic symptoms. |
| Lower extremity | The LEFS is used to monitor perceived lower limb functional capacity throughout the |
| functional scale | study. The LEFS is expressed as a score out of 80, the lower the score, the greater the |
| (LEFS) | level of disability. LEFS is self-administered and participants are allowed to ask any |
| | questions to better understand the questionnaire if needed. |

### Example 11—Multi-Center Confirmatory Study in Human Patients with Adult-Onset Clinically Demonstratable DM1

Approximately 60 subjects with adult-onset clinically demonstratable DM1 are enrolled and treated with study drug (HSA-IGF2-R61A or placebo) for 12 weeks. Endpoints to be evaluated would include those related to function (mobility, strength), muscle mass, pulmonary function, patient reported outcomes, cognitive function, metabolic markers, quality of life/activities of daily living, biomarkers associated with target engagement and myogenesis, in addition to monitoring safety and tolerability. This study is projected to enroll approximately 150 subjects. Study drug (HSA-IGF2-R61A or placebo) is administered for approximately 6 months. Endpoints included are similar to those evaluated in the Phase 2 study.

While preferred aspects of the present invention have been shown and described herein, it will be obvious to those skilled in the art that such aspects are provided by way of example only. Numerous variations, changes, and substitutions will now occur to those skilled in the art without departing from the invention. It should be understood that various alternatives to the aspects of the invention described herein may be employed in practicing the invention. It is intended that the following claims define the scope of the invention and that methods and structures within the scope of these claims and their equivalents be covered thereby.

What is claimed is:

1. A pharmaceutical composition comprising:
a fusion protein comprising the amino acid sequence of SEQ ID NO: 1 and a stabilizer, wherein the stabilizer is present at a mass concentration of solution of about 0.5% to 15% weight/volume (w/v) of the pharmaceutical composition.

2. A pharmaceutical composition comprising:
a fusion protein comprising the amino acid sequence of SEQ ID NO: 1 and a stabilizer, wherein the stabilizer is present at a mass concentration of solution of at least about 5% w/v of the pharmaceutical composition.

3. A pharmaceutical composition comprising:
a fusion protein comprising the amino acid sequence of SEQ ID NO: 1 and a stabilizer, wherein the stabilizer is present at a mass concentration of solution of less than about 10% w/v of the pharmaceutical composition.

4. The pharmaceutical composition of claim 1, wherein the stabilizer is present at a mass concentration of solution about 8% w/v of the pharmaceutical composition.

5. The pharmaceutical composition of any one of claims 1-4, wherein the stabilizer is a carbohydrate.

6. The pharmaceutical composition of claim 5, wherein the carbohydrate comprises sucrose, mannitol, trehalose, raffinose, stachyose, xylitol, starch, verbascose, or any combination thereof.

7. The pharmaceutical composition of claim 5 or 6, wherein the carbohydrate comprises sucrose.

8. The pharmaceutical composition of any one of claims 1-7, wherein the stabilizer is present at a mass concentration of solution between about 5 to 10% w/v of the pharmaceutical composition.

9. The pharmaceutical composition of claim 8, wherein sucrose is present at a mass concentration of solution between about 5 to 10% w/v of the pharmaceutical composition.

10. The pharmaceutical composition of claim 8 or 9, wherein sucrose is present at a mass concentration of solution of about 8% w/v of the pharmaceutical composition.

11. A pharmaceutical composition comprising:
a fusion protein comprising the amino acid sequence of SEQ ID NO: 1 and a surfactant, wherein the surfactant is present at a mass concentration of solution of about 0.001% to 1% w/v of the pharmaceutical composition.

12. A pharmaceutical composition comprising:
a fusion protein comprising the amino acid sequence of SEQ ID NO: 1 and a surfactant, wherein the surfactant is present at a mass concentration of solution of at least about 0.01% w/v of the pharmaceutical composition.

13. A pharmaceutical composition comprising:
a fusion protein comprising the amino acid sequence of SEQ ID NO: 1 and a surfactant, wherein the surfactant is present at a mass concentration of solution of less than about 1% w/v of the pharmaceutical composition.

14. The pharmaceutical composition of any one of claims 11-13, wherein the surfactant is present at a mass concentration of solution between about 0.05-0.20% w/v of the pharmaceutical composition.

15. The pharmaceutical composition of any one of claims 11-14, wherein the surfactant is present at a mass concentration of solution between about 0.01% w/v of the pharmaceutical composition.

16. The pharmaceutical composition of any one of claims 11-15, wherein the surfactant is an ionic surfactant.

17. The pharmaceutical composition of any one of claims 11-15, wherein the surfactant is a nonionic surfactant.

18. The pharmaceutical composition of claim 17, wherein the nonionic surfactant comprises a polysorbate or a poloxamer.

19. The pharmaceutical composition of claim 17 or 18, wherein the nonionic surfactant comprises a polysorbate comprising Tween 20, Tween 40, Tween 60, Tween 80, Tween 65, Tween 80, or any combination thereof.

20. The pharmaceutical composition of claim 17 or 18, wherein the nonionic surfactant comprises a poloxamer comprising poloxamer 124, poloxamer 188, poloxamer 237, poloxamer 338, poloxamer 407, or any combination thereof.

21. The pharmaceutical composition of any one of claims 17-20, wherein the nonionic surfactant comprises poloxamer 188.

22. The pharmaceutical composition of any one of claims 17-21, wherein the nonionic surfactant comprises poloxamer 188 at a mass concentration of solution of about 0.1% w/v.

23. The pharmaceutical composition of claim 22, wherein the nonionic surfactant consists of poloxamer 188.

24. A pharmaceutical composition comprising:
a fusion protein comprising the amino acid sequence of SEQ ID NO: 1 and a buffer, wherein the buffer is present at a concentration of about 1 mM to about 100 mM.

25. The pharmaceutical composition of claim 24, wherein the buffer comprises sodium, potassium, calcium, or ammonium.

26. The pharmaceutical composition of claim 24 or 25, wherein the buffer comprises sodium and wherein the sodium is present at a concentration of about 3 mM to about 10 mM.

27. The pharmaceutical composition of any one of claims 24-26, wherein the buffer comprises sodium and wherein the sodium is present at a concentration of about 7 nM.

28. The pharmaceutical composition of any one of claims 1-10, or 24-27, further comprising a surfactant.

29. The pharmaceutical composition of claim 28, wherein the surfactant is an ionic surfactant.

30. The pharmaceutical composition of claim 28, wherein the surfactant is a nonionic surfactant.

31. The pharmaceutical composition of any one of claims 28-30, wherein the surfactant is present at a concentration of about 0.001-1% w/v of the pharmaceutical composition.

32. The pharmaceutical composition of any one of claims 28-31, wherein the surfactant is present at a concentration of about 0.1% w/v of the pharmaceutical composition.

33. The pharmaceutical composition of any one of claims 30-32, wherein the nonionic surfactant comprises a polysorbate or a poloxamer.

34. The pharmaceutical composition of claim 33, wherein the nonionic surfactant comprises a poloxamer comprising poloxamer 124, poloxamer 188, poloxamer 237, poloxamer 338, poloxamer 407, or any combination thereof.

35. The pharmaceutical composition of any one of claims 30-34, wherein the nonionic surfactant comprises poloxamer 188.

36. The pharmaceutical composition of any one of claims 30-35, wherein the nonionic surfactant comprises poloxamer 188 at a mass concentration of solution of about 0.1% w/v.

37. The pharmaceutical composition of any one of claims 11-36, further comprising a stabilizer, wherein the stabilizer is present at a mass concentration of solution of about 0.5% to 15% weight/volume (w/v) of the pharmaceutical composition.

38. The pharmaceutical composition of any one of claims 11-36, further comprising a stabilizer, wherein the stabilizer is present at a mass concentration of solution of at least about 5% w/v of the pharmaceutical composition.

39. The pharmaceutical composition of any one of claims 11-36, further comprising a stabilizer, wherein the stabilizer is present at a mass concentration of solution of less than about 10% w/v of the pharmaceutical composition.

40. The pharmaceutical composition of any one of claims 11-39, wherein the stabilizer is present at a mass concentration of solution between about 5 to 10% w/v of the pharmaceutical composition.

41. The pharmaceutical composition of any one of claims 11-40, wherein the stabilizer is present at a mass concentration of solution about 8% w/v of the pharmaceutical composition.

42. The pharmaceutical composition of any one of claims 11-41, wherein the stabilizer is a carbohydrate.

43. The pharmaceutical composition of claim 42, wherein the carbohydrate comprises sucrose, mannitol, trehalose, raffinose, stachyose, xylitol, starch, verbascose, or any combination thereof.

44. The pharmaceutical composition of claim 42 or 43, wherein the carbohydrate comprises sucrose.

45. The pharmaceutical composition of claim 43 or 44, wherein sucrose is present at a mass concentration of solution between about 5 to 10% w/v of the pharmaceutical composition.

46. The pharmaceutical composition of any one of claims 43-45, wherein sucrose is present at a mass concentration of solution of about 8% w/v of the pharmaceutical composition.

47. The pharmaceutical composition of any one of claims 1-23, or 28-46, further comprising a salt, wherein the salt is present at a concentration of about 1 mM to about 100 mM.

48. The pharmaceutical composition of claim 47, wherein the salt comprises sodium, potassium, calcium, or ammonium.

49. The pharmaceutical composition of claim 47 or 48, wherein the salt comprises sodium and wherein the sodium is present at a concentration of about 3 mM to about 10 mM.

50. The pharmaceutical composition of any one of claims 47-49, wherein the salt comprises sodium and wherein the sodium is present at a concentration of about 7 nM.

51. The pharmaceutical composition of any one of claims 1-50, wherein the pharmaceutical composition is formulated for subcutaneous administration.

52. The pharmaceutical composition of claim 51, wherein the fusion protein is formulated in a dosage form of injectable liquid having a volume of about 0.1 mL to about 3 mL, or about 0.5 mL to about 2 mL, or about 0.1 mL to about 1 mL.

53. The pharmaceutical composition of any one of claims 1-52, wherein the fusion protein is present at a concentration of about 30 mg/mL to about 350 mg/mL.

54. The pharmaceutical composition of any one of claims 1-53, wherein the fusion protein is present at a concentration of about 100 mg/mL to about 200 mg/mL.

55. A pharmaceutical composition comprising:
a fusion protein comprising the amino acid sequence of SEQ ID NO: 1; and
a pharmaceutically acceptable excipient, carrier, or diluent;
wherein the pharmaceutical composition is formulated for subcutaneous administration.

56. The pharmaceutical composition of claim 55, wherein the fusion protein is formulated in a dosage form of injectable liquid having a volume of about 0.1 mL to about 3 mL, or about 0.5 mL to about 2 mL, or about 0.1 mL to about 1 mL.

57. The pharmaceutical composition of claim 55 or 56, wherein the fusion protein is present at a concentration of about 30 mg/mL to about 350 mg/mL.

58. The pharmaceutical composition of any one of claims 55-57, wherein the fusion protein is present at a concentration of about 100 mg/mL to about 200 mg/mL.

59. A pharmaceutical composition comprising a fusion protein comprising the amino acid sequence of SEQ ID NO: 1, wherein the fusion protein is present at a concentration of about 30 mg/mL to about 350 mg/mL.

60. A pharmaceutical composition comprising a fusion protein comprising the amino acid sequence of SEQ ID NO: 1, wherein the fusion protein is present at a concentration of about 100 mg/mL to about 200 mg/mL.

61. The pharmaceutical composition of claim 59 or 60, formulated in a subcutaneous unit dosage form.

62. The pharmaceutical composition of claim 61, formulated in a dosage form of injectable liquid having a volume of about 0.1 mL to about 3 mL, or about 0.5 mL to about 2 mL, or about 0.1 mL to about 1 mL.

63. The pharmaceutical composition of any one of claims 55-62, further comprising a surfactant.

64. The pharmaceutical composition of claim 63, wherein the surfactant is an ionic surfactant.

65. The pharmaceutical composition of claim 63, wherein the surfactant is a nonionic surfactant.

66. The pharmaceutical composition of any one of claims 63-65, wherein the surfactant is present at a concentration of about 0.001-1% w/v of the pharmaceutical composition.

67. The pharmaceutical composition of any one of claims 63-66, wherein the surfactant is present at a concentration of about 0.1% w/v of the pharmaceutical composition.

68. The pharmaceutical composition of any one of claims 65-67, wherein the nonionic surfactant comprises a polysorbate or a poloxamer.

69. The pharmaceutical composition of claim 68, wherein the nonionic surfactant comprises a poloxamer comprising poloxamer 124, poloxamer 188, poloxamer 237, poloxamer 338, poloxamer 407, or any combination thereof.

70. The pharmaceutical composition of any one of claims 65-69, wherein the nonionic surfactant comprises poloxamer 188.

71. The pharmaceutical composition of any one of claims 65-70, wherein the nonionic surfactant comprises poloxamer 188 at a mass concentration of solution of about 0.1% w/v.

72. The pharmaceutical composition of any one of claims 55-71, further comprising a stabilizer, wherein the stabilizer is present at a mass concentration of solution of about 0.5% to 15% weight/volume (w/v) of the pharmaceutical composition.

73. The pharmaceutical composition of any one of claims 55-71, further comprising a stabilizer, wherein the stabilizer is present at a mass concentration of solution of at least about 5% w/v of the pharmaceutical composition.

74. The pharmaceutical composition of any one of claims 55-71, further comprising a stabilizer, wherein the stabilizer is present at a mass concentration of solution of less than about 10% w/v of the pharmaceutical composition.

75. The pharmaceutical composition of any one of claims 72-74, wherein the stabilizer is present at a mass concentration of solution between about 5 to 10% w/v of the pharmaceutical composition.

76. The pharmaceutical composition of any one of claims 72-75, wherein the stabilizer is present at a mass concentration of solution about 8% w/v of the pharmaceutical composition.

77. The pharmaceutical composition of any one of claims 72-76, wherein the stabilizer is a carbohydrate.

78. The pharmaceutical composition of claim 77, wherein the carbohydrate comprises sucrose, mannitol, trehalose, raffinose, stachyose, xylitol, starch, verbascose, or any combination thereof.

79. The pharmaceutical composition of claim 77 or 78 wherein the carbohydrate comprises sucrose.

80. The pharmaceutical composition of claim 78 or 79, wherein sucrose is present at a mass concentration of solution between about 5 to 10% w/v of the pharmaceutical composition.

81. The pharmaceutical composition of any one of claims 77-80, wherein sucrose is present at a mass concentration of solution of about 8% w/v of the pharmaceutical composition.

82. The pharmaceutical composition of any one of claims 55-81, further comprising a salt, wherein the salt is present at a concentration of about 1 mM to about 100 mM.

83. The pharmaceutical composition of claim 82, wherein the salt comprises sodium, potassium, calcium, or ammonium.

84. The pharmaceutical composition of claim 82 or 83, wherein the salt comprises sodium and wherein the sodium is present at a concentration of about 3 mM to about 10 mM.

85. The pharmaceutical composition of any one of claims 82-84, wherein the salt comprises sodium and wherein the sodium is present at a concentration of about 7 nM.

86. The pharmaceutical composition of any one of claims 1-85, further comprising a buffering agent.

87. The pharmaceutical composition of any one of claims 1-86, wherein the pharmaceutical composition has a pH less than 7.5.

88. The pharmaceutical composition of any one of claims 1-87, wherein the pharmaceutical composition has a pH greater than 4.5.

89. The pharmaceutical composition of any one of claims 1-88, wherein the pharmaceutical composition has a pH of about 4.5 to about 7.5.

90. The pharmaceutical composition of any one of claims 1-89, wherein the pharmaceutical composition has a pH of about 5.5 to about 6.5.

91. The pharmaceutical composition of any one of claims 1-90, wherein the pharmaceutical composition has a pH of about 6.0.

92. The pharmaceutical composition of any one of claims 86-91, wherein the buffering agent comprises sucrose octa-sulfate, ammonium carbonate, ammonium phosphate, boric acid, sodium citrate, potassium citrate, lactic acid, 3-(N-morpholino)propanesulfonic acid (MOPS), 2-(N-morpholino)ethanesulfonic acid (MES), hydroxymethylaminomethane (Tris), calcium carbonate, calcium phosphate, or any combination thereof.

93. The pharmaceutical composition of any one of claims 86-92, wherein the buffering agent comprises sodium citrate at a concentration of about 3 mM to about 10 mM.

94. The pharmaceutical composition of any one of claims 86-90, or 92-93, wherein the buffering agent comprises sodium citrate at a pH of about 5.5 to about 6.5.

95. The pharmaceutical composition of any one of claims 86-94, wherein the buffering agent comprises sodium citrate at a concentration of about 7 mM.

96. The pharmaceutical composition of any one of claims 86-95, wherein the buffering agent comprises sodium citrate at a concentration of about 7 mM and a pH of about 6.0.

97. A method of treating a subject having myotonic muscular dystrophy, comprising administering an effective amount of a fusion protein comprising the amino acid sequence of SEQ ID NO: 1 to the subject, wherein the subject has received a physician diagnosis of myotonic dystrophy type 1 (DM1) or myotonic dystrophy type 2 (DM2).

98. A method of treating a subject having myotonic muscular dystrophy, comprising administering an effective amount of a fusion protein comprising the amino acid sequence of SEQ ID NO: 1 to the subject, wherein the subject has received a physician diagnosis of myotonic dystrophy type 1 (DM1).

99. A method of treating a subject having myotonic muscular dystrophy, comprising administering an effective amount of a fusion protein comprising the amino acid sequence of SEQ ID NO: 1 to the subject, wherein the subject has received a physician diagnosis of myotonic dystrophy type 2 (DM2).

100. The method of any one of claims 97-99, wherein the administering comprises subcutaneous administration.

101. The method of any one of claims 97-100, wherein the administering comprises administration according to a dosage regime.

102. The method of claim 101, wherein the dosage regime comprises once daily, once every other day, twice weekly, once weekly, once every ten days, once every two weeks, once every three weeks, once every month, once every two months, once every three months, once every four months, or once every six months administration of the fusion protein to the subject.

103. The method of any one of claims 97-102, wherein the pharmaceutical composition of any one of claims 1-96 is administered once daily.

104. The method of any one of claims 97-102, wherein the pharmaceutical composition of any one of claims 1-96 is administered once every other day.

105. The method of any one of claims 97-102, wherein the pharmaceutical composition of any one of claims 1-96 is administered twice weekly.

106. The method of any one of claims 97-102, wherein the pharmaceutical composition of any one of claims 1-96 is administered weekly.

107. The method of any one of claims 97-102, wherein the pharmaceutical composition of any one of claims 1-96 is administered every other week.

108. The method of any one of claims 97-107, wherein the effective amount of the fusion protein is less than about 0.5, 0.75, 1, 1.25, 1.5, 1.75, 2.0, 2.25, 2.5, 2.75, 3, 3.5, 4, 4.5, or 5 mg/kg of body weight of the subject.

109. The method of any one of claims 97-107, wherein the effective amount of the fusion protein is greater than about 0.01, 0.02, 0.05, 0.1, 0.2, 0.3, 0.4, 0.5, 0.6, 0.7, 0.75, 0.8, 0.9, 1, 1.1, 1.25, 1.35, 1.5, 1.65, 1.75, 1.85, 2.0, 2.25, 2.5, 2.75, or 3 mg/kg of body weight of the subject.

110. The method of any one of claims 97-109, wherein the effective amount of the fusion protein is between about 0.5 to about 4 mg/kg of body weight of the subject.

111. The method of any one of claims 97-110, wherein the effective amount of the fusion protein is between about 1 to about 3 mg/kg of body weight of the subject.

112. The method of any one of claims 97-111, wherein the effective amount of the fusion protein administered to the subject is adjusted to achieve a peak serum concentration (Cₘₐₓ) following subcutaneous administration of less than or equal to about 50, 40, 30, 25, 20, 17.5, 15, 12.5, 10, 7.5, 5 or 3 μg/mL.

113. The method of any one of claims 97-112, wherein the effective amount of the fusion protein administered to the subject is adjusted to achieve a peak serum concentration (Cₘₐₓ) following subcutaneous administration of less than or equal to 15 μg/mL.

114. The method of any one of claims 97-111, wherein the effective amount of the fusion protein administered to the subject is adjusted to achieve a peak serum concentration (Cₘₐₓ) following subcutaneous administration of greater than or equal to about 1, 2, 3, 4, 5, 6, 7, 8, 9, 10, 11, 12, 13, 14, 15, 16, 17, 18, 19, 20, 22.5, 25, 27.5, or 30 μg/mL.

115. The method of any one of claims 97-112, or 114, wherein the effective amount of the fusion protein administered to the subject is adjusted to achieve a peak serum concentration (Cₘₐₓ) following subcutaneous administration of between about 5 to about 25 μg/mL.

116. The method of any one of claims 97-98, or 100-115, wherein the subject receiving a physician diagnosis of DM1 has received genetic confirmation of DMPK repeat expansion.

117. The method of claim 116, wherein the DMPK repeat expansion comprises greater than 100 CTG repeats in the DMPK gene.

118. The method of any one of claims 97-117, wherein the fusion protein agonistically promotes ERK1, ERK2, or ERK1 and ERK2 phosphorylation following the subcutaneous administration to the subject.

119. The method of claim 118, wherein promotion of ERK1, ERK2, or ERK1 and ERK2 phosphorylation activates ERK1/2 downstream signaling following the subcutaneous administration to the subject.

120. The method of any one of claims 97-119, wherein the fusion protein agonistically promotes mitogen-activated protein kinase kinase (MEK) pathway activation following the subcutaneous administration to the subject.

121. The method of any one of claims 97-120, wherein the fusion protein agonistically activates PI3K signaling following the subcutaneous administration to the subject.

122. The method of any one of claims 97-121, wherein the fusion protein agonistically activates AKT signaling following the subcutaneous administration to the subject.

123. The method of any one of claims 97-122, wherein the activation of PI3K signaling agonistically activates AKT signaling following the subcutaneous administration to the subject.

124. The method of any one of claims 97-123, wherein the administering promotes muscle cell differentiation in the subject.

125. The method of any one of claims 97-124, wherein the administering promotes muscle cell survival in the subject.

126. The method of claim 125, wherein the promotion of muscle cell survival in the subject leads to a lower rate of muscle cell degeneration compared to a subject having myotonic muscular dystrophy that has not been administered the fusion protein.

127. The method of any one of claims 97-126, wherein the administering improves muscle strength in the subject.

128. The method of any one of claims 97-127, wherein the administering reduces muscle wasting in the subject.

129. The method of any one of claims 97-128, wherein the administering comprises subcutaneous injection in the subject.

130. The method of any one of claims 103-129, wherein the pharmaceutical composition is administered with a syringe that contains 0.02 mL graduations.

131. The method of any one of claims 97-130, wherein the subject is at least 11 years old.

132. The method of any one of claims 97-131, wherein dosage of the fusion protein administered to the subject per administration is about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3 mL of the pharmaceutical composition of any one of claims 1-96.

133. The method of claim 132, wherein dosage of the fusion protein administered to the subject is determined based on actual body of the subject at initiation of therapy.

134. The method of any one of claims 97-133, wherein the subject possesses an ability to walk independently for at least 10 meters at a time of screening prior to initiation of administration of the fusion protein.

135. The method of claim 134, wherein the subject is assessed for the ability to walk independently with or without the use of orthoses or ankle braces.

136. A method of treating a subject having myotonic muscular dystrophy, comprising administering to the subject a pharmaceutical composition comprising a fusion protein comprising the amino acid sequence of SEQ ID NO: 1, wherein the pharmaceutical composition is administered to the subject at a dose of at least about 5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 210, 225, 240, 250, 260, 275, 290, 300, 325, 350, 375, 400, 425, 450 mg of the fusion protein.

137. A method of treating a subject having myotonic muscular dystrophy, comprising administering to the subject a pharmaceutical composition comprising a fusion protein comprising the amino acid sequence of SEQ ID NO: 1, wherein the pharmaceutical composition is administered to the subject at a dose of at most about 10, 15, 20, 25, 30, 35, 40, 45, 50, 55, 60, 65, 70, 75, 80, 85, 90, 95, 100, 110, 120, 125, 130, 140, 150, 160, 170, 175, 180, 190, 200, 210, 225, 240, 250, 260, 275, 290, 300, 325, 350, 375, 400, 425, 450, or 500 mg of the fusion protein.

138. A method of treating a subject having myotonic muscular dystrophy, comprising administering to the subject a pharmaceutical composition comprising a fusion protein comprising the amino acid sequence of SEQ ID NO: 1, wherein the pharmaceutical composition is administered to the subject at a dose of at most about 5 mg to 500 mg of the fusion protein.

139. The method of any one of claims 136-138, wherein the pharmaceutical composition is administered subcutaneously.

140. The method of any one of claim 136-139, wherein the subject is administered the pharmaceutical composition of any one of claims 1-96 according to the schedule of a dosage regime.

141. The method of claim 140, wherein the dosage regime comprises once daily, once every other day, twice weekly, once weekly, once every ten days, once every two weeks, once every three weeks, once every month, once every two months, once every three months, once every four months, or once every six months administration of the fusion protein to the subject.

142. The method of claim 140 or 141, wherein the pharmaceutical composition of any one of claims 1-96 is administered once daily.

143. The method of claim 140 or 141, wherein the pharmaceutical composition of any one of claims 1-96 is administered once every other day.

144. The method of claim 140 or 141, wherein the pharmaceutical composition of any one of claims 1-96 is administered twice weekly.

145. The method of claim 140 or 141, wherein the pharmaceutical composition of any one of claims 1-96 is administered weekly.

146. The method of claim 140 or 141, wherein the pharmaceutical composition of any one of claims 1-96 is administered every other week.

147. The method of any one of claims 136-146, wherein an effective amount of the fusion protein to treat the subject is less than about 0.5, 0.75, 1, 1.25, 1.5, 1.75, 2.0, 2.25, 2.5, 2.75, 3, 3.5, 4, 4.5, or 5 mg/kg of body weight of the subject.

148. The method of any one of claims 136-146, wherein an effective amount of the fusion protein to treat the subject is greater than about 0.01, 0.02, 0.05, 0.1, 0.2, 0.3, 0.4, 0.5, 0.6, 0.7, 0.75, 0.8, 0.9, 1, 1.1, 1.25, 1.35, 1.5, 1.65, 1.75, 1.85, 2.0, 2.25, 2.5, 2.75, or 3 mg/kg of body weight of the subject.

149. The method of any one of claims 136-146, wherein an effective amount of the fusion protein to treat the subject is between about 0.5 to about 4 mg/kg of body weight of the subject.

150. The method of any one of claims 136-146, wherein an effective amount of the fusion protein to treat the subject is between about 1 to about 3 mg/kg of body weight of the subject.

151. The method of any one of claims 136-150, wherein the amount of the fusion protein administered to the subject is adjusted to achieve a peak serum concentration (Cₘₐₓ) following subcutaneous administration of less than or equal to about 50, 40, 30, 25, 20, 17.5, 15, 12.5, 10, 7.5, 5 or 3 μg/mL.

152. The method of any one of claims 136-151, wherein the amount of the fusion protein administered to the subject is adjusted to achieve a peak serum concentration (Cₘₐₓ) following subcutaneous administration of less than or equal to 15 μg/mL.

153. The method of any one of claims 136-150, wherein the amount of the fusion protein administered to the subject is adjusted to achieve a peak serum concentration (Cₘax) following subcutaneous administration of greater than or equal to about 1, 2, 3, 4, 5, 6, 7, 8, 9, 10, 11, 12, 13, 14, 15, 16, 17, 18, 19, 20, 22.5, 25, 27.5, or 30 μg/mL.

154. The method of any one of claims 136-151, or 153, wherein the effective amount of the fusion protein administered to the subject is adjusted to achieve a peak serum concentration (Cₘₐₓ) following subcutaneous administration of between about 5 to about 25 μg/mL.

155. The method of any one of claims 136-154, wherein the subject has received a physician diagnosis of myotonic dystrophy type 1 (DM1) or myotonic dystrophy type 2 (DM2).

156. The method of any one of claims 136-155, wherein the subject has received a physician diagnosis of myotonic dystrophy type 1 (DM1).

157. The method of any one of claims 136-155, wherein the subject has received a physician diagnosis of myotonic dystrophy type 2 (DM2).

158. The method of claim 155 or 156, wherein the subject receiving a physician diagnosis of DM1 has received genetic confirmation of DMPK repeat expansion.

159. The method of claim 158, wherein the DMPK repeat expansion comprises greater than 100 CTG repeats in the DMPK gene.

160. The method of any one of claims 136-159, wherein the subject possesses an ability to walk independently for at least 10 meters at a time of screening prior to initiation of administration of the fusion protein.

161. The method of claim 160, wherein the subject is assessed for the ability to walk independently with or without the use of orthoses or ankle braces.

162. A kit comprising the pharmaceutical composition of any one of claims 1-96, and instructions for use.

163. The kit of claim 162, wherein the instructions for use designate myotonic muscular dystrophy as an indication in a subject in need of treatment.

164. The kit of claim 162, wherein the instructions for use designate adult-onset myotonic dystrophy type 1 (adult-onset DM1) as an indication in a subject in need of treatment.

165. The kit of any one of claims 162-164, further comprising a drug delivery device for administering the pharmaceutical composition to a subject.

166. The kit of claim 165, wherein the drug delivery device is a pre-filled syringe.

167. The kit of claim 166, wherein the pre-filled syringe has a set volume of about 0.4 mL, about 0.8 mL, about 1.0 mL, about 1.2 mL, or about 1.6 mL.

168. The kit of claim 166 or 167, wherein the pre-filled syringe is configured to deliver a dosage of about 0.4 mg/mL, about 0.5 mg/mL, about 1.0 mg/mL, about 1.5 mg/mL, or about 2.0 mg/mL.

169. The kit of any one of claims 166-168, wherein the pre-filled syringe contains a 100 mg/mL formulation concentration of the pharmaceutical composition.
